(19) United States
(12) Patent Application Publication
(10) Pub. No.: US 2011/0065706 A1
(43) Pub. Date: Mar. 17, 2011

(54) Therapeutic Agents 812

(75) Inventors: Alan Martin Birch, Cheshire (GB); Roger John Butlin, Cheshire (GB); David Stephen Clarke, Cheshire (GB); Andrew Leach, Cheshire (GB); Philip Alexander MacFaul, Cheshire (GB); Charles John O'Donnell, Cheshire (GB); James Stewart Scott, Cheshire (GB); Paul Robert Owen Whittamore, Cheshire (GB); Dan Anders Broo, Molndal (SE); Öjvind Percy Davidsson, Molndal (SE); Kjell Erik Johansson, Molndal (SE); Hanna De La Motte, Sodertalje (SE)

(73) Assignee: AstraZeneca AB, Sodertalje (SE)

(21) Appl. No.: 12/879,631

(22) Filed: Sep. 10, 2010

Related U.S. Application Data
(60) Provisional application No. 61/241,545, filed on Sep. 11, 2009.
(60) Provisional application No. 61/304,564, filed on Feb. 15, 2010.

(51) Int. Cl. A61K 31/5377; C07D 403/04; A61K 31/506; C07D 403/14; C07D 401/14; C07D 413/14; C07D 487/08; C07D 405/14; A61P 3/04; A61P 3/10

(52) U.S. Cl. 514/235.8; 544/295; 514/252.14; 514/252.19; 514/252.18; 544/296; 544/335; 514/273; 514/252.11; 544/121; 514/252.2

(57) ABSTRACT

A compound of formula I

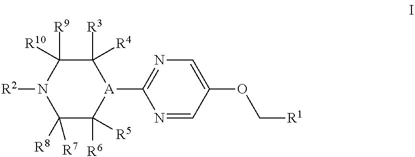

or a pharmaceutically acceptable salt thereof, processes for preparing such compounds, their use as GPR119 modulators, methods for their therapeutic use, particularly in the treatment of obesity and diabetes mellitus, and pharmaceutical compositions containing them.

This application claims the benefit under 35 U.S.C. §119(e) of Application Nos. 61/241,545 (US) filed on 11 Sep. 2009 and 61/304564 filed on 15 Feb. 2010.

### FIELD OF INVENTION

The present invention relates to certain 1-substituted-4-(5-(phenyl or heteroaryl)methoxy)-pyrimidin-2-yl)piperazines and piperidines to processes for preparing such compounds, to their use as GPR119 modulators (particularly agonists), to methods for their therapeutic use, particularly in the treatment of obesity and diabetes mellitus, and to pharmaceutical compositions containing them.

### BACKGROUND OF THE INVENTION

Obesity and diabetes are reaching epidemic proportions in the USA, EU, Japan and developing countries. Obesity is the major driver of the co-morbidities of the metabolic syndrome, particularly type 2 diabetes. Since no effective pharmacotherapies for obesity is are available to date and current diabetes therapies do not stop the progression of the disease, there is a huge unmet medical need.

GPR119 (also known as HRUP, RUP3, GDIR, 19AJ, OSGPR116, SNORF25) is a class A Gs coupled receptor. Originally an orphan receptor, it has recently been de-orphanised, with the natural ligands believed to be oleoylethanolamide (OEA) and lysophosphatidylcholine.

It is believed that the receptor is expressed in the pancreas, and in the K cells (GIP secreting) and L cells (GLP-1 secreting) located in the gut. Although GPR119 expression is not seen in human brain, there are high levels expressed in a number of regions of rat and mouse brain.

It is expected that orally active GPR119 modulators, particularly agonists, will potentiate glucose stimulated insulin secretion, either directly due to pancreatic GPR119 agonism, or indirectly by stimulation of GLP-1/GIP release, and so improve long term glycaemic control. Furthermore, long term preservation of beta cell mass is also a possibility as a result of the increased cAMP concentrations in beta cells induced either directly or as a result of increased GLP-1 secretion. Finally, GPR119 agonists have also been reported to reduce food intake in rodent models.

1-[3-[(3,4-Dihydro-4-oxo-1-phthalazinyl)methyl]benzoyl]-4-[5-(phenylmethoxy)-2-pyrimidinyl]piperazine is disclosed as a PARP inhibitor useful in the treatment of cancer in US20050059663 and WO 2004080976. (3R,4R)-rel-3-Hydroxy-4-[5-(phenylmethoxy)-2-pyrimidinyl]-1-piperidinecarboxylic acid 1,1-dimethylethyl ester is disclosed as an intermediate in WO 2002/076440 and WO 9709311. 2-[4-(4-Azolylbutyl)-1-piperazinyl-5-hydroxypyrimidine derivatives and their use as CNS agents are disclosed in U.S. Pat. No. 5,444,059. (1R*, 2S*, 3R*, 4S*)-N-[4-[4-[5-(Phenylmethoxy)-2-pyrimidinyl]-1-piperazinyl]butyl]-2,3-bicyclo[2.2.1]heptanedicarboximide is disclosed as an intermediate and as antianxiety agent in U.S. Pat. No. 4,423,049 and in Chemical & Pharmaceutical Bulletin (1991), 39(9), 2288-2300. 4,4-Dimethyl-1-[4-[4-[5-(phenylmethoxy)-2-pyrimidinyl]-1-piperazinyl]butyl]-2,6-piperidinedione is disclosed as an intermediate in U.S. Pat. No. 4,843,078. Pyrimidopiperazines are disclosed in WO2005/011657 as inhibitors of human stearoyl-CoA desaturase.

GPR119 agonists are disclosed in WO2009038974, WO2010009183 and WO2010008739.

### DESCRIPTION OF THE INVENTION

The present invention provides a compound of formula I

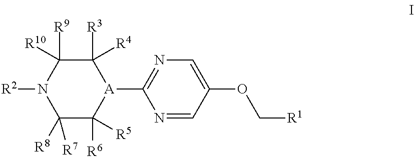

or a pharmaceutically acceptable salt thereof in which
A represents N or CH;
R¹ represents a) a phenyl ring substituted in the 4-position by one of the groups 1 to 6 below and wherein the phenyl ring is optionally additionally substituted in the 2 and/or the 3 and/or the 5 and/or the 6 position by a group independently selected one or more of the following: cyano, fluoro, hydroxy, a C₁₋₄cycloalkoxy, a C₁₋₄alkoxy optionally substituted by one or more fluoro or a C₁₋₄alkyl optionally substituted by hydroxy or C₁₋₄alkoxy or by one or more fluoro;
1) a group —N(R¹¹)COR¹² in which R¹¹ represents H or a C₁₋₆alkyl optionally substituted by one or more of the following: fluoro, hydroxy or C₁₋₄alkoxy and R¹² represents a C₁₋₆alkyl optionally substituted by one or more of the following: fluoro, hydroxy, a C₃₋₆cycloalkyl, C₁₋₄alkoxy or a group —NR¹³R¹⁴ in which R¹³ and R¹⁴ independently represent H, a C₁₋₆alkyl optionally substituted by one or more of the following: fluoro, hydroxy or C₁₋₄alkoxy or R¹² represents a C₃₋₆cycloalkyl optionally substituted by one or more of the following: fluoro, hydroxy, C₁₋₄alkyl or C₁₋₄alkoxy or R¹² represents a group (CH₂)ₖ-Het wherein k is 0,1, 2, 3 or 4 and Het represents a carbon linked saturated 4 to 7 membered heterocyclic group containing one or more N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
2) a group —CONR¹⁵R¹⁶ in which R¹⁵ and R¹⁶ independently represent H, C₃₋₆cycloalkyl or a C₁₋₆alkyl optionally substituted by one or more of the following i) fluoro ii) hydroxy iii) is C₁₋₄alkoxy
iv) C₃₋₆cycloalkyl or v) a group —NR¹⁷R¹⁸ in which R¹⁷ and R¹⁸ independently represent H or a C₁₋₆alkyl optionally substituted by one or more of the following: fluoro, hydroxy, C₁₋₄alkyl or C₁₋₄alkoxy; or R¹⁷ and R¹⁸ together with the nitrogen to which they are attached represent a saturated 4-7 membered heterocyclic group optionally containing an additional N, S or O and wherein the heterocyclic group is optionally substituted by one or more of the following: fluoro, hydroxy, oxo, C₁₋₄alkyl or C₁₋₄alkoxy; v) a carbon linked saturated 4 to 7 membered heterocyclic group containing one or more N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy; or R¹⁵ and R¹⁶ together with the nitrogen to which they are attached represent a saturated 4-7 membered heterocyclic group optionally containing an additional N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
3) a group —(CH₂)ₗ—(O)ₘS(O)ₙR¹⁹ in which m is 0 or 1 and when m is 0 then 1 is 0, 1, 2, 3, or 4 and n is 1 or 2 and when m is 1 then 1 is 0 and n is 2 and R¹⁹ represents a C₁₋₆alkyl optionally substituted by one or more of the following: fluoro, hydroxy, C₃₋₆cycloalkyl, C₁₋₄alkyl or C₁₋₄alkoxy; or by a group —NR²⁰R²¹ in which R²⁰ and R²¹ independently represent H, C₃₋₆cycloalkyl or a C₁₋₆alkyl or R²⁰ and R²¹ together with the nitrogen to which they are attached represent a saturated 4-7 membered heterocyclic group optionally containing an additional N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
or R¹⁹ represents C₃₋₆cycloalkyl optionally substituted by one or more of the following: fluoro, hydroxy, C₁₋₄alkyl or C₁₋₄alkoxy;
or R¹⁹ represents a carbon linked saturated 4 to 7 membered heterocyclic group containing one or more N, S or O wherein the S may be in its oxidised form of SO or SO₂ , and
wherein the heterocyclic group is optionally substituted by one or more of the following: fluoro, hydroxy, oxo, C₁₋₄alkyl or C₁₋₄alkoxy;
4) a group —N(R²²)CON(R²³)(R²⁴) in which R²², R²³ and R²⁴ independently represent H or a C₁₋₆alkyl group;
5) a group SO₂NR²⁵R²⁶ in which R²⁵ and R²⁶ independently represent H, a C₁₋₆alkyl group or a C₃₋₆cycloalkyl group wherein the alkyl and cycloalkyl groups are optionally substituted by one or more of the following: fluoro, hydroxy, C₁₋₄alkyl or C₁₋₄alkoxy;
or R²⁵ and R²⁶ together with the nitrogen to which they are attached represent a saturated 4-7 membered heterocyclic group optionally containing an additional N, S or O and wherein the heterocyclic group is optionally substituted by one or more of the following: fluoro, hydroxy, oxo, C₁₋₄alkyl or C₁₋₄alkoxy;
6) a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy;
or R¹ represents
b) 4-pyridyl optionally substituted by one or more of the following: halo, cyano, C₁₋₄alkyl, C₁₋₄alkoxy, C₁₋₄alkylsulfonyl or a group CONR²⁷R²⁸ in which R²⁷ and R²⁸ independently represent H or a C₁₋₆alkyl group; or
c) 2-pyridyl substituted in the 5-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the pyridyl ring is optionally additionally substituted by one or more of the following: halo, cyano, C₁₋₄alkyl or C₁₋₄alkoxy; or
d) 3-pyridyl substituted in the 6-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the pyridyl ring is optionally additionally substituted by one or is more of the following: halo, cyano, C₁₋₄alkyl or C₁₋₄alkoxy; or
e) pyrimidin-4-yl or pyrimidin-5-yl optionally substituted in the 2 position by a C₁₋₆alkanoylamino group or by cyano;
R² represents 1) a group —CO—OR^{x} in which R^{x} represents a C₁₋₆alkyl optionally substituted by cyano, hydroxy, C₁₋₄alkoxy or by one or more fluoro or R^{x} represents C₃₋₆cycloalkyl optionally substituted by one or more of the following: fluoro, hydroxy, C₁₋₄alkyl or C₁₋₄alkoxy; or R^{x} represents a saturated cyclic ether containing an oxygen and 3, 4 or 5 carbons optionally substituted by one or more of the following: fluoro, hydroxy, C₁₋₄alkyl optionally substituted by one or more fluoro or C₁₋₄alkoxy optionally substituted by one or more fluoro;
2) 2-pyrimidyl optionally substituted by one or more of the following: cyano, one or more halo, C₁₋₄alkoxy which is optionally substituted by one or more fluoro, C₃₋₆cycloalkyl optionally substituted by one or more of the following: fluoro, hydroxy, C₁₋₄alkyl or C₁₋₄alkoxy; or C₁₋₄alkyl which is optionally substituted by one or more of the following: fluoro, hydroxy, C₁₋₄alkyl or C₁₋₄alkoxy; or
3) 1,2,4-oxadiazol-3-yl or 1,2,4-oxadiazol-5-yl each of which is optionally substituted by cyano, by one or more halo, by C₁₋₄alkoxy which is optionally substituted by one or more fluoro, by C₃₋₆cycloalkyl optionally substituted by one or more of the following: fluoro, hydroxy, C₁₋₄alkyl or C₁₋₄alkoxy; or by C₁₋₄alkyl which is optionally substituted by one or more of the following: fluoro, hydroxy, C₁₋₄alkyl or C₁₋₄alkoxy;
R³, R⁴, R⁵, R⁶, R⁷, R⁸, R⁹ and R¹⁰ independently represent H or a C₁₋₄alkyl group optionally substituted by one or more of the following: fluoro, hydroxy, C₁₋₄alkyl or C₁₋₄alkoxy; or R³ and R⁷ together represent a methylene or ethylene bridge, or R⁷ and R⁹ together represent a methylene or ethylene bridge, or R³ and R⁵ together represent a methylene or ethylene bridge; and additionally when A is CH then R³ and R⁵ may also be independently selected from fluoro, hydroxy, or C₁₋₄alkoxy.

In one group of compounds of formula I, A is N.

The present invention also provides a compound of formula I

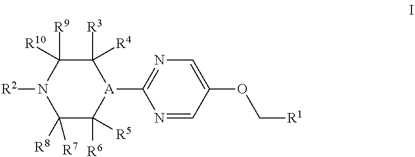

or a pharmaceutically acceptable salt thereof in which
A represents N or CH;
R¹ represents a) a phenyl ring substituted in the 4-position by one of the groups 1 to 6 below and wherein the phenyl ring is optionally additionally substituted in the 2 and/or the 3 and/or the 5 and/or the 6 position by a group independently selected one or more of the following: cyano, fluoro, hydroxy, a C₃₋₄cycloalkoxy, a C₁₋₄alkoxy optionally substituted by one or more fluoro or a C₁₋₄alkyl optionally substituted by hydroxy or C₁₋₄alkoxy or by one or more fluoro;
1) a group —N(R¹¹)COR¹² in which R¹¹ represents H or a C₁₋₆alkyl optionally substituted by one or more of the following: fluoro, hydroxy or C₁₋₄alkoxy and R¹² represents a C₁₋₆alkyl optionally substituted by one or more of the following: fluoro, hydroxy, a C₃₋₆cycloalkyl, C₁₋₄alkoxy or a group —NR¹³R¹⁴ in which R¹³ and R¹⁴ independently represent H, a C₁₋₆alkyl optionally substituted by one or more of the following: fluoro, hydroxy or C₁₋₄alkoxy or R¹² represents a C₃₋₆cycloalkyl optionally substituted by one or more of the following: fluoro, hydroxy, C₁₋₄alkyl or C₁₋₄alkoxy or R¹² represents a group (CH₂)ₖ-Het wherein k is 0,1, 2, 3 or 4 and Het represents a carbon linked saturated 4 to 7 membered heterocyclic group containing one or more N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
2) a group —CONR¹⁵R¹⁶ in which R¹⁵ and R¹⁶ independently represent H, C₃₋₆cycloalkyl or a C₁₋₆alkyl optionally substituted by one or more of the following i) fluoro ii) hydroxy iii) C₁₋₄alkoxy
iv) C₃₋₆cycloalkyl or v) a group —NR¹⁷R¹⁸ in which R¹⁷ and R¹⁸ independently represent H or a C₁₋₆alkyl optionally substituted by one or more of the following: fluoro, hydroxy, C₁₋₄alkyl or C₁₋₄alkoxy; or R¹⁷ and R¹⁸ together with the nitrogen to which they are attached represent a saturated 4-7 membered heterocyclic group optionally containing an additional N, S or O and wherein the heterocyclic group is optionally substituted by one or more of the following: fluoro, hydroxy, oxo, C₁₋₄alkyl or C₁₋₄alkoxy; v) a carbon linked saturated 4 to 7 membered heterocyclic group containing one or more N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy; or R¹⁵ and R¹⁶ together with the nitrogen to which they are attached represent a saturated 4-7 membered heterocyclic group optionally containing an additional N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
3) a group —(CH₂)ₗ—(O)ₘS(O)ₙR¹⁹ in which m is 0 or 1 and when m is 0 then 1 is 0, 1, 2, 3, or 4 and n is 1 or 2 and when m is 1 then 1 is 0 and n is 2 and R¹⁹ represents a C₁₋₆alkyl optionally substituted by one or more of the following: fluoro, hydroxy, C₃₋₆cycloalkyl, C₁₋₄alkyl or C₁₋₄alkoxy; or by a group —NR²⁰R²¹ in which R²⁰ and R²¹ independently represent H, C₃₋₆cycloalkyl or a C₁₋₆alkyl or R²⁰ and R²¹ together with the nitrogen to which they are attached represent a saturated 4-7 membered heterocyclic group optionally containing an additional N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
or R¹⁹ represents C₃₋₆cycloalkyl optionally substituted by one or more of the following: fluoro, hydroxy, C₁₋₄alkyl or C₁₋₄alkoxy;
or R¹⁹ represents a carbon linked saturated 4 to 7 membered heterocyclic group containing one or more N, S or O wherein the S may be in its oxidised form of SO or SO₂ , and
wherein the heterocyclic group is optionally substituted by one or more of the following: fluoro, hydroxy, oxo, C₁₋₄alkyl or C₁₋₄alkoxy;
4) a group —N(R²²)CON(R²³)(R²⁴) in which R²², R²³ and R²⁴ independently represent H or a C₁₋₆alkyl group;
5) a group SO₂NR²⁵R²⁶ in which R²⁵ and R²⁶ independently represent H, a C₁₋₆alkyl group or a C₃₋₆cycloalkyl group wherein the alkyl and cycloalkyl groups are optionally substituted by one or more of the following: fluoro, hydroxy, C₁₋₄alkyl or C₁₋₄alkoxy; or R²⁵ and R²⁶ together with the nitrogen to which they are attached represent a saturated 4-7 membered heterocyclic group optionally containing an additional N, S or O and wherein the heterocyclic group is optionally substituted by one or more of the following: fluoro, hydroxy, oxo, C₁₋₄alkyl or C₁₋₄alkoxy;
6) a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy;
or R¹ represents
b) 4-pyridyl optionally substituted by one or more of the following: halo, cyano, C₁₋₄alkyl, C₁₋₄alkoxy, C₁₋₄alkylsulfonyl or a group CONR²⁷R²⁸ in which R²⁷ and R²⁸ independently represent H or a C₁₋₆alkyl group; or
c) 2-pyridyl substituted in the 5-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the pyridyl ring is optionally additionally substituted by one or more of the following: halo, cyano, C₁₋₄alkyl or C₁₋₄alkoxy; or
d) 3-pyridyl substituted in the 6-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the pyridyl ring is optionally additionally substituted by one or more of the following: halo, cyano, C₁₋₄alkyl or C₁₋₄alkoxy; or
e) pyrimidin-4-yl or pyrimidin-5-yl optionally substituted in the 2 position by a C₁₋₆alkanoylamino group or by cyano;
R² represents 1) a group —CO—OR^{x} in which R^{x} represents a C₁₋₆alkyl optionally substituted by cyano, hydroxy, C₁₋₄alkoxy or by one or more fluoro or R^{x} represents C₃₋₆cycloalkyl optionally substituted by one or more of the following: fluoro, hydroxy, C₁₋₄alkyl or C₁₋₄alkoxy; or R^{x} represents a saturated cyclic ether containing an oxygen and 3, 4 or 5 carbons optionally substituted by one or more of the following: fluoro, hydroxy, C₁₋₄alkyl or C₁₋₄alkoxy;
2) 2-pyrimidyl optionally substituted by one or more of the following: cyano, one or more halo, C₁₋₄alkoxy which is optionally substituted by one or more fluoro, C₃₋₆cycloalkyl optionally substituted by one or more of the following: fluoro, hydroxy, C₁₋₄alkyl or C₁₋₄alkoxy; or C₁₋₄alkyl which is optionally substituted by one or more of the following: fluoro, hydroxy, C₁₋₄alkyl or C₁₋₄alkoxy; or
3) 1,2,4-oxadiazol-3-yl or 1,2,4-oxadiazol-5-yl each of which is optionally substituted by cyano, by one or more halo, by C₁₋₄alkoxy which is optionally substituted by one or more fluoro, by C₃₋₆cycloalkyl optionally substituted by one or more of the following: fluoro, hydroxy, C₁₋₄alkyl or C₁₋₄alkoxy; or by C₁₋₄alkyl which is optionally substituted by one or more of the following: fluoro, hydroxy, C₁₋₄alkyl or C₁₋₄alkoxy;
R³, R⁴, R⁵, R⁶, R⁷, R⁸, R⁹ and R¹⁰ independently represent H or a C₁₋₄alkyl group optionally substituted by one or more of the following: fluoro, hydroxy, C₁₋₄alkyl or C₁₋₄alkoxy; or R³ and R⁷ together represent a methylene or ethylene bridge, or R⁷ and R⁹ together represent a methylene or ethylene bridge, or R³ and R⁵ together represent a methylene or ethylene bridge; and additionally when A is CH then R³ and R⁵ may also be independently selected from fluoro, hydroxy or C₁₋₄alkoxy.

When R¹ is phenyl the 1-position of the phenyl ring is the point of attachment to * in the —O—(CH₂)—* group in formula I. When R¹ is pyridyl then the pyridyl nitrogen is numbered as 1 and the point of attachment to * in the —O—(CH₂)—* group in formula I is given the lowest appropriate number and other substituents are numbered accordingly. Similarly when R¹ is pyrimidyl then a nitrogen of the pyrimidine is numbered as 1, the other nitrogen of the pyrimidine is numbered as 3 and the point of attachment to * in the —O—(CH₂)—* is group in formula I is given the next lowest number. This is illustrated in formulae II and III below.

In one group of compounds of formula I, A is N.

In a further aspect the present invention provides a compound of formula II

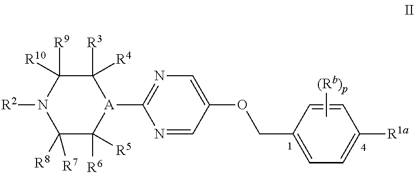

or a pharmaceutically acceptable salt thereof in which
A represents N or CH;
R^{1a} represents a group selected from one of groups 1-6 in R¹ above;
p=0 or 1 and R^{b} fluoro or C₁₋₃alkyl;
and R², R³, R⁴, R⁵, R⁶, R⁷, R⁸, R⁹ and R¹⁰ are as described above.

In one group of compounds of formula II, A is N.

In a further aspect the present invention provides a compound of formula III

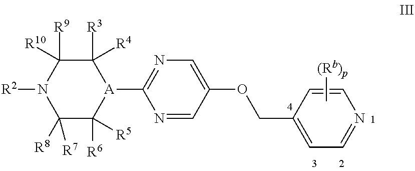

or a pharmaceutically acceptable salt thereof in which
A represents N or CH;
p=0, 1 or 2; R^{b} is bromo, fluoro, cyano, C₁₋₄alkoxy or C₁₋₄alkyl, and R², R³, R⁴, R⁵, R⁶, R⁷, R⁸, R⁹ and R¹⁰ are as described above.

In one group of compounds of formula III, A is N.

In one aspect the present invention provides a compound of formula I in which R¹ represents 2-pyridyl substituted in the 5-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the 2-pyridyl ring is optionally additionally substituted by one or more of the following: halo, cyano, C₁₋₄alkyl or C₁₋₄alkoxy; and A, R², R³, R⁴, R⁵, R⁶, R⁷, R⁸, R⁹ and R¹⁰ are as described above. In one particular group of such compounds, A is N.

In another aspect the present invention provides a compound of formula I in which R¹ represents 3-pyridyl substituted in the 6-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the 3-pyridyl ring is optionally additionally substituted by one or more of the following: halo, cyano, C₁₋₄alkyl or C₁₋₄alkoxy; and A, R², R³, R⁴, R⁵, R⁶, R⁷, R⁸, R⁹ and R¹⁰ are as described a particular group of such compounds, A is N.

In another aspect the present invention provides a compound of formula IV

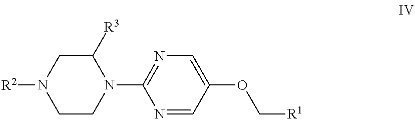

or a pharmaceutically acceptable salt thereof in which
R¹ represents phenyl substituted in the 4-position by a C₁₋₄alkylsulfonyl group or by a C₁₋₄alkylsulfonyloxy group and optionally the phenyl is additionally substituted by fluoro; or R¹ represents 4-pyridyl optionally substituted by cyano;
R² represents a group —CO—OR^{x} in which R^{x} represents a C₁₋₆alkyl optionally substituted by cyano or by one or more fluoro or R² represents 2-pyrimidyl optionally substituted by halo; and
R³ represents H or methyl.

In another aspect the present invention provides a compound of formula IV

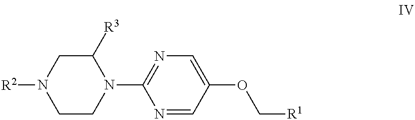

or a pharmaceutically acceptable salt thereof in which
R¹ represents phenyl substituted in the 4-position by a C₁₋₄alkylsulfonyl group or by a C₁₋₄alkylsulfonyloxy group or by a C₁₋₄alkylsulfonylC₁₋₄alkyl group and optionally the phenyl is additionally substituted by fluoro;
or R¹ represents 4-pyridyl optionally substituted by cyano;
R² represents a group —CO—OR^{x} in which R^{x} represents oxetanyl optionally substituted by trifluoromethyl or by methyl;
R³ represents H or methyl.

In another aspect the present invention provides a compound of formula IV

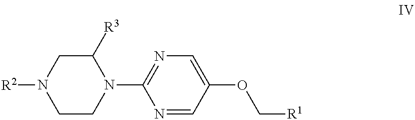

R¹ represents phenyl substituted in the 4-position by a C₁₋₄alkylsulfonyl group or by a C₁₋₄alkylsulfonyloxy group or by a C₁₋₄alkylsulfonylC₁₋₄alkyl group and optionally the phenyl is additionally substituted by fluoro; or R¹ represents 4-pyridyl optionally substituted by cyano;
R² represents 1,2,4-oxadiazol-3-yl or 1,2,4-oxadiazol-5-yl each of which is optionally substituted by a C₁₋₄alkyl group which is optionally substituted by C₁₋₄alkoxy or by one or more fluoro or R² represents a group —COOR^{x} wherein R^{x} represents a C₁₋₄alkyl group optionally substituted by one or more fluoro or R^{x} represents oxetan-3-yl optionally substituted by a C₁₋₄alkyl group which is optionally substituted by one or more fluoro; and
R³ represents methyl.

In another aspect the present invention provides a compound of formula IV

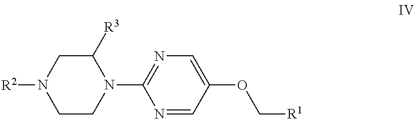

R¹ represents 3-cyanopyridin-4-yl, 2-fluoro-4-(methylsulfonyl)phenyl or 2-fluoro-4-(methylsulfonylmethyl)phenyl;
R² represents 5-isopropyl-1,2,4-oxadiazol-3-yl, 3-isopropyl-1,2,4-oxadiazol-5-yl, 3-(trifluoromethyl)-1,2,4-oxadiazol-5-yl, 5-((S)-1-methoxyethyl)-1,2,4-oxadiazol-3-yl, 5-((R)-1-methoxyethyl)-1,2,4-oxadiazol-3-yl, or a group —COOR^{x} wherein R^{x} is (R)-1,1,1-trifluoropropan-2-yl), (S)-1,1,1-trifluoropropan-2-yl or (R)-3-(trifluoromethyl)oxetan-3-yl: and
R³ represents methyl.

Preferred values of each variable group are as follows. Such values may be used where appropriate with any of the values, definitions, claims, aspects or embodiments defined hereinbefore or hereinafter. In particular, each may be used as an individual limitation on the broadest definition of formula (I) (including formulae II , III and IV). Further, each of the following values may be used in combination with one or more of the other following values to limit the broadest definition, or any sub-definition, of formula (I).

R¹ represents a phenyl ring substituted in the 4-position by a group selected from —N(R¹¹)COR¹² in which R¹¹ represents H or a C₁₋₆alkyl optionally substituted by one or more of the following: fluoro, hydroxy or C₁₋₄alkoxy and R¹² represents a C₁₋₆alkyl optionally substituted by one or more of the following: fluoro, hydroxy, C₁₋₄alkoxy or a group —NR¹³R¹⁴ in which R¹³ and R¹⁴ independently represent H, a C₁₋₆alkyl optionally substituted by one or more of the following: fluoro, hydroxy or C₁₋₄alkoxy or or R¹² represents a C₃₋₆cycloalkyl optionally substituted by one or more of the following: fluoro, hydroxy, C₁₋₄alkyl or C₁₋₄alkoxy or R¹² represents a carbon linked saturated 4 to 7 membered heterocyclic group containing one or more N, S or O, wherein the S may be in is its oxidised form of SO or SO₂ wherein the heterocyclic group is optionally substituted by one or more of the following: fluoro, hydroxy, C₁₋₄alkyl or C₁₋₄alkoxy; wherein the phenyl ring is optionally additionally substituted in the 2 or 3 or 5 or 6 position by a group independently selected one or more of the following: cyano, fluoro, hydroxy, a C₃₋₄cycloalkoxy, C₁₋₄alkoxy or a C₁₋₄alkyl optionally substituted by fluoro, hydroxy or C₁₋₄alkoxy.

R¹ represents a phenyl ring substituted in the 4-position by a group selected from —CONR¹⁵R¹⁶ in which R¹⁵ and R¹⁶ independently represent H, a C₁₋₆alkyl optionally substituted by one or more of the following i) fluoro ii) hydroxy iii) C₁₋₄alkoxy iv) a group —NR¹⁷R¹⁸ in which R¹⁷ and R¹⁸ independently represent H or a C₁₋₆alkyl optionally substituted by one or more of the following: fluoro, hydroxy, C₁₋₄alkyl or C₁₋₄alkoxy; or R¹⁷ and R¹⁸ together with the nitrogen to which they are attached represent a saturated 4-7 membered heterocyclic group optionally containing an additional N, S or O, and optionally substituted by one or more of the following: fluoro, hydroxy, C₁₋₄alkyl or C₁₋₄alkoxy; v) a carbon linked saturated 4 to 7 membered heterocyclic group containing one or more N, S or O, wherein the S may be in its oxidised form of SO or SO₂ , optionally substituted by one or more of the following: fluoro, hydroxy, C₁₋₄alkyl or C₁₋₄alkoxy; or R¹⁵ and R¹⁶ together with the nitrogen to which they are attached represent a saturated 4-7 membered heterocyclic group optionally containing an additional N, S or O, and optionally substituted by one or more of the following: fluoro, hydroxy, C₁₋₄alkyl or C₁₋₄alkoxy; wherein the phenyl ring is optionally additionally substituted in the 2 or 3 or 5 or 6 position by a group independently selected one or more of the following: fluoro, hydroxy, C₁₋₄alkoxy or a C₁₋₄alkyl optionally substituted by fluoro, hydroxy or C₁₋₄alkoxy.

R¹ represents a phenyl ring substituted in the 4-position by a group selected from —(O)ₘS(O)ₙR¹⁹ in which m is 0 or 1 and when m is 0 then n is 1 or 2 and when m is 1 then n is 2 and R¹⁹ represents a C₁₋₆alkyl optionally substituted by one or more of the following: fluoro, hydroxy, C₁₋₄alkyl or C₁₋₄alkoxy; or by a group —NR²⁰R²¹ in which R²⁰ and R²¹ independently represent H or a C₁₋₆alkyl or R²⁰ and R²¹ together with the nitrogen to which they are attached represent a saturated 4-7 membered heterocyclic group optionally containing an additional N, S or O, and optionally substituted by one or more of the following: fluoro, hydroxy, C₁₋₄alkyl or C₁₋₄alkoxy; or R¹⁹ represents C₃₋₆cycloalkyl optionally substituted by one or more of the following: fluoro, hydroxy, C₁₋₄alkyl or C₁₋₄alkoxy; or R¹⁹ represents a carbon linked saturated 4 to 7 membered heterocyclic group containing one or more N, S or O, wherein the S may be in its oxidised form of SO or SO₂, optionally substituted by one or more of the following: fluoro, hydroxy, C₁₋₄alkyl or C₁₋₄alkoxy; wherein the phenyl ring is optionally additionally substituted in the 2 or 3 or 5 or 6 position by a group independently selected one or more of the following: fluoro, hydroxy, C₁₋₄alkoxy or a C₁₋₄alkyl optionally substituted by fluoro, hydroxy or C₁₋₄alkoxy.

R¹ represents a phenyl ring substituted in the 4-position by a group selected from —N(R²²)CON(R²³)(R²⁴) in which R²², R²³ and R²⁴ independently represent H or a C₁₋₆alkyl group; wherein the phenyl ring is optionally additionally substituted in the 2 or 3 or 5 or 6 position by a group independently selected one or more of the following: fluoro, hydroxy, C₁₋₄alkoxy or a C₁₋₄alkyl optionally substituted by fluoro, hydroxy or C₁₋₄alkoxy.

R¹ represents a phenyl ring substituted in the 4-position by a group selected from—a group SO₂NR²⁵R²⁶ in which R²⁵ and R²⁶ independently represent H, a C₁₋₆alkyl group or a C₃₋₆cycloalkyl group wherein the alkyl and cycloalkyl groups are optionally substituted by one or more of the following: fluoro, hydroxy, C₁₋₄alkyl or C₁₋₄alkoxy;
or R²⁵ and R²⁶ together with the nitrogen to which they are attached represent a saturated 4-7 membered heterocyclic group optionally containing an additional N, S or O, and optionally substituted by one or more of the following: fluoro, hydroxy, C₁₋₄alkyl or C₁₋₄alkoxy; wherein the phenyl ring is optionally additionally substituted in the 2 or 3 or 5 or 6 position by a group independently selected one or more of the following: fluoro, hydroxy, C₁₋₄alkoxy or a C₁₋₄alkyl optionally substituted by fluoro, hydroxy or C₁₋₄alkoxy.

R¹ represents a phenyl ring substituted in the 4-position by a group selected from a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; wherein the phenyl ring is optionally additionally substituted in the 2 or 3 or 5 or 6 position by a group independently selected one or more of the following: fluoro, hydroxy, C₁₋₄alkoxy or a C₁₋₄alkyl optionally substituted by fluoro, hydroxy or C₁₋₄alkoxy.

R¹ represents 4-pyridyl optionally substituted by one or more of the following: halo, cyano, C₁₋₄alkyl, C₁₋₄alkoxy, C₁₋₄alkylsulfonyl or a group CONR²⁷R²⁸ in which R²⁷ and R²⁸ independently represent H or a C₁₋₆alkyl group.

R¹ represents 2-pyridyl substituted in the 5-position by C₁₋₄alkylsulfonyl, C₂₋₄alkanoylamino or by a 5-membered heteroaromatic group containing 1, 2, 3 or 4 hetero is atoms selected from O, N and S optionally substituted by one or more of the following: fluoro, hydroxy, C₁₋₄alkyl or C₁₋₄alkoxy; and wherein either pyridyl ring is optionally additionally substituted by one or more of the following: halo, cyano, C₁₋₄alkyl or C₁₋₄alkoxy.

R¹ represents 3-pyridyl substituted in the 6-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein either pyridyl ring is optionally additionally substituted by one or more of the following: halo, cyano, C₁₋₄alkyl or C₁₋₄alkoxy.

R¹ represents pyrimidin-4-yl or pyrimidin-5-yl optionally substituted in the 2 position by a C₁₋₆alkanoylamino group.

R¹ represents 2-fluoro-4-methylsulfonylphenyl, 2-methyl-4-methylsulfonylphenyl, 3-fluoro-4-methylsulfonylphenyl, 4-cyclopropylsulfonylphenyl, 4-ethylsulfonylphenyl, 4-methylsulfinylphenyl, 4-(trifluoromethylsulfinyl)phenyl, 4-(2-morpholinoethylsulfonyl)-phenyl, 4-methylsulfonyloxyphenyl, 4-(methylsulfamoyl)phenyl, 3-methyl-4-(2-methyl-propanoylamino)phenyl, 3-methyl-4-(tert-butylcarbamoyl)phenyl, 3-methyl-4-methylsulfonylphenyl, 4-(2,2-dimethylpropanoylamino)-3-methylphenyl, 4-(2,2-dimethylpropanoylamino)phenyl, 4-(N-(2-dimethylaminoethyl)carbamoyl)phenyl, 4-(N-(2-dimethylaminoethyl)-N-methyl-carbamoyl)phenyl, 4-(N-(2-hydroxyethyl)carbamoyl)-phenyl, 4-(N-2-hydroxyethyl-(N-methyl)-carbamoyl)phenyl, 4-(N-2-hydroxyethyl-(N-methyl)carbamoyl)phenyl, 4-(2-methylpropanoylamino)phenyl, 4-(4-methylpiperazine-1-carbonyl)phenyl, 4-(cyclopentanecarbonylamino)phenyl, 4-(isopropylcarbamoyl)-3-methylphenyl, 4-(isopropylcarbamoyl)phenyl, 4-(isopropylcarbamoylamino)phenyl, 4-(methylcarbamoyl)phenyl, 4-(morpholine-4-carbonyl)phenyl, 4-(piperazine-1-carbonyl)phenyl, 4-(tert-butylcarbamoyl)phenyl, 4-[(1-methyl-4-piperidyl)methyl-carbamoyl]phenyl, 4-acetamidophenyl, 4-(1,2,4-triazol-1-yl)phenyl, 4-(5-methyl-1,2,4-oxadiazol-3-yl)phenyl, 4-(tetrazol-1-yl)phenyl, 4-pyridyl, 3-cyano-4-pyridyl, 3-bromo-4-pyridyl, 3-methoxy-4-pyridyl, 3-methyl-4-pyridyl, 5-fluoro-2-methoxy-4-pyridyl, 5-methylsulfonyl-2-pyridyl, 6-acetamido-3-pyridyl, 6-methylsulfonyl-3-pyridyl, 6-(1,2,4-triazol-1-yl)-3-pyridyl, 6-pyrazol-1-yl-3-pyridyl, pyrimidin-4-yl, or 2-(2-methylpropanoylamino)pyrimidin-5-yl.

R¹ represents 2-fluoro-4-methylsulfonylphenyl, 2-fluoro-4-methylsulfonylmethylphenyl, 2-methyl-4-methylsulfonylphenyl, 3-fluoro-4-methylsulfonylphenyl, 4-cyclopropylsulfonylphenyl, 4-ethylsulfonylphenyl, 4-methylsulfinylphenyl, 4-(trifluoromethylsulfinyl)phenyl, 4-(2-morpholinoethylsulfonyl)-phenyl, 4-methylsulfonyloxyphenyl, 4-(methylsulfamoyl)phenyl, 3-methyl-4-(2-methylpropanoylamino)phenyl, 3-methyl-4-(tert-butylcarbamoyl)phenyl, 3-methyl-4-methylsulfonylphenyl, 4-(2,2-dimethylpropanoylamino)-3-methylphenyl, 4-(2,2-dimethylpropanoylamino)phenyl, 4-(2-dimethylaminoethylcarbamoyl)phenyl, 4-(N-(2-dimethylaminoethyl)-N-methyl-carbamoyl)phenyl, 4-(2-hydroxyethylcarbamoyl)phenyl, 4-(N-2-hydroxyethyl-(N-methyl)carbamoyl)phenyl, 4-(2-methylpropanoylamino)phenyl, 4-(4-methylpiperazine-1-carbonyl)phenyl, 4-(cyclopentanecarbonylamino)phenyl, 4-(isopropylcarbamoyl)-3-methylphenyl, 4-(isopropylcarbamoyl)phenyl, 4-(isopropylcarbamoylamino)phenyl, 4-(methylcarbamoyl)phenyl, 4-(morpholine-4-carbonyl)phenyl, 4-(piperazine-1-carbonyl)phenyl, 4-(tert-butylcarbamoyl)phenyl, 4-[N-(1-methyl-4-piperidyl)methyl)carbamoyl]phenyl or 4-acetamidophenyl.

R¹ represents 4-(1,2,4-triazol-1-yl)phenyl, 4-(5-methyl-1,2,4-oxadiazol-3-yl)phenyl or 4-(tetrazol-1-yl)phenyl.

R¹ represents 4-pyridyl, 3-cyano-4-pyridyl, 3-bromo-4-pyridyl, 3-methoxy-4-pyridyl, 3-methyl-4-pyridyl, 5-fluoro-2-methoxy-4-pyridyl, 5-methylsulfonyl-2-pyridyl, 6-acetamido-3-pyridyl, 6-methylsulfonyl-3-pyridyl.

R¹ represents 4-pyridyl, 3-cyano-4-pyridyl, 3-bromo-4-pyridyl, 3-methoxy-4-pyridyl, 3-methyl-4-pyridyl, 5-fluoro-2-methoxy-4-pyridyl, 5-methylsulfonyl-2-pyridyl, 6-acetamido-3-pyridyl, 6-methylsulfonyl-3-pyridyl, 6-(1,2,4-triazol-1-yl)-3-pyridyl, 6-pyrazol-1-yl-3-pyridyl, 6-acetamido-3-pyridyl or 6-methylsulfonyl-3-pyridyl. R¹ represents 4-pyridyl, 3-cyano-4-pyridyl, 3-bromo-4-pyridyl, 3-methoxy-4-pyridyl, 3-methyl-4-pyridyl or 5-fluoro-2-methoxy-4-pyridyl.

R¹ represents 6-(1,2,4-triazol-1-yl)-3-pyridyl, 6-pyrazol-1-yl-3-pyridyl, 6-acetamido-3-pyridyl or 6-methylsulfonyl-3-pyridyl.

R¹ represents 5-methylsulfonyl-2-pyridyl.

R¹ represents pyrimidin-4-yl or 2-(2-methylpropanoylamino)pyrimidin-5-yl.

R¹ represents 4-methylsulfonyloxyphenyl, 3-cyano-4-pyridyl or 2-fluoro-4-methylsulfonylphenyl.

R² represents a group —CO—OR^{x} in which R^{x} represents a C₁₋₆alkyl optionally substituted by cyano, hydroxy, C₁₋₄alkoxy or by one or more fluoro or R^{x} represents C₃₋₆cycloalkyl optionally substituted by one or more of the following: fluoro, hydroxy, C₁₋₄alkyl or C₁₋₄alkoxy; or R^{x} represents a saturated cyclic ether containing an oxygen and 3, 4 or 5 carbons optionally substituted by one or more of the following: fluoro, hydroxy, C₁₋₄alkyl optionally substituted by one or more fluoro or C₁₋₄alkoxy optionally substituted by one or more fluoro.

R² represents a group —CO—OR^{x} in which R^{x} represents a C₁₋₆alkyl optionally substituted by cyano, hydroxy, C₁₋₄alkoxy or by one or more fluoro or R^{x} represents C₃₋₆cycloalkyl optionally substituted by one or more of the following: fluoro, hydroxy, C₁₋₄alkyl or C₁₋₄alkoxy; or R^{x} represents a saturated cyclic ether containing an oxygen and 3, 4 or 5 carbons optionally substituted by one or more of the following: fluoro, hydroxy, C₁₋₄alkyl or C₁₋₄alkoxy.

R² represents a group —CO—OR^{x} in which R^{x} represents (2-, 3- or 4-oxetanyl optionally substituted by one or more of the following: methyl or trifluoromethyl. R² represents a group —CO—OR^{x} in which R^{x} represents 3-oxetanyl optionally substituted by one or more of the following: methyl or trifluoromethyl.

R² represents 2-pyrimidyl optionally substituted by one or more halo.

R² represents 1,2,4-oxadiazol-3-yl or 1,2,4-oxadiazol-5-yl each of which is optionally substituted by a C₃₋₆cycloalkyl group or by a C₁₋₄alkyl group which is optionally substituted by one or more fluoro.

R² represents 1,2,4-oxadiazol-3-yl or 1,2,4-oxadiazol-5-yl each of which is optionally substituted by a C₁₋₄alkyl group which is optionally substituted by one or more fluoro.

R² represents (2,2,2-trifluoro-1-methyl-ethoxy)carbonyl, (1-cyano-1-methylethoxy)carbonyl), tert-butoxycarbonyl, (1-methylcyclopropoxy)carbonyl, cyclobutoxycarbonyl, isopropoxycarbonyl, (3-methyloxetan-3-yl)oxycarbonyl, oxetan-3-yloxycarbonyl, tetrahydrofuran-3-yloxycarbonyl, tetrahydropyran-4-yloxycarbonyl, 5-(trifluoromethyl)-1,2,4-oxadiazol-3-yl, 5-isopropyl-1,2,4-oxadiazol-3-yl or 5-fluoropyrimidin-2-yl.

R² represents (2,2,2-trifluoro-1-methyl-ethoxy)carbonyl, (1-cyano-1-methylethoxy)carbonyl), tert-butoxycarbonyl, 5-fluoropyrimidin-2-yl, 5-((S)-1-methoxyethyl)-1,2,4-oxadiazol-3-yl or 5-((R)-1-methoxyethyl)-1,2,4-oxadiazol-3-yl.

R² represents (2,2,2-trifluoro-1-methyl-ethoxy)carbonyl, (1-cyano-1-methylethoxy)carbonyl), tert-butoxycarbonyl, (1-methylcyclopropoxy)carbonyl, cyclobutoxycarbonyl, isopropoxycarbonyl, (3-methyloxetan-3-yl)oxycarbonyl, oxetan-3-yloxycarbonyl, tetrahydrofuran-3-yloxycarbonyl, tetrahydropyran-4-yloxycarbonyl, 5-(trifluoromethyl)-1,2,4-oxadiazol-3-yl, 5-isopropyl-1,2,4-oxadiazol-3-yl or 5-fluoropyrimidin-2-yl.

R² represents (2,2,2-trifluoro-1-methyl-ethoxy)carbonyl, (1-cyano-1-methylethoxy)carbonyl), tert-butoxycarbonyl or 5-fluoropyrimidin-2-yl. R² represents (2,2,2-trifluoro-1-methyl-ethoxy)carbonyl, (1-cyano-1-methylethoxy)-carbonyl), tert-butoxycarbonyl, (1-methylcyclopropoxy)carbonyl, cyclobutoxycarbonyl, isopropoxycarbonyl, (3-methyloxetan-3-yl)oxycarbonyl, oxetan-3-yloxycarbonyl, tetrahydrofuran-3-yloxycarbonyl or tetrahydropyran-4-yloxycarbonyl.

R² represents (2,2,2-trifluoro-1-methyl-ethoxy)carbonyl, (1-cyano-1-methylethoxy)carbonyl), tert-butoxycarbonyl, (1-methylcyclopropoxy)carbonyl, cyclobutoxycarbonyl, isopropoxycarbonyl, (3-methyloxetan-3-yl)oxycarbonyl, oxetan-3-yloxycarbonyl, tetrahydrofuran-3-yloxycarbonyl, tetrahydropyran-4-yloxycarbonyl, (R)-1,1,1-trifluoropropan-2-yloxycarbonyl, (S)-1,1,1-trifluoropropan-2-yloxycarbonyl, 2,2,2-trifluoroethoxycarbonyl or 3-(trifluoromethyl)oxetan-3-yloxycarbonyl.

R² represents 5-(trifluoromethyl)-1,2,4-oxadiazol-3-yl, 5-isopropyl-1,2,4-oxadiazol-3-yl, 5-fluoropyrimidin-2-yl, 5-(difluoromethyl)-1,2,4-oxadiazol-3-yl, 5-cyclopropyl-1,2,4-oxadiazol-3-yl, 3-isopropyl-1,2,4-oxadiazol-5-yl, 2-methyl-4-(3-(trifluoromethyl)-1,2,4-oxadiazol-5-yl, 5-((S)-1-methoxyethyl)-1,2,4-oxadiazol-3-yl or 5-((R)-1-methoxyethyl)-1,2,4-oxadiazol-3-yl.

R² represents 5-(trifluoromethyl)-1,2,4-oxadiazol-3-yl, 5-isopropyl-1,2,4-oxadiazol-3-yl, 5-fluoropyrimidin-2-yl, 5-(difluoromethyl)-1,2,4-oxadiazol-3-yl, 5-cyclopropyl-1,2,4-oxadiazol-3-yl, 3-isopropyl-1,2,4-oxadiazol-5-yl or 2-methyl-4-(3-(trifluoromethyl)-1,2,4-oxadiazol-5-yl.

R² represents 5-(trifluoromethyl)-1,2,4-oxadiazol-3-yl, 5-isopropyl-1,2,4-oxadiazol-3-yl or 5-fluoropyrimidin-2-yl.

R³, R⁴, R⁵, R⁶, R⁷, R⁸, R⁹ and R¹⁰ independently represent H or a C₁₋₄alkyl group optionally substituted by one or more of the following: fluoro, hydroxy, C₁₋₄alkyl or C₁₋₄alkoxy; or R³ and R⁷ together represent a methylene or ethylene bridge, or R⁷ and R⁹ together represent a methylene or ethylene bridge, or R³ and R⁵ together represent a methylene or ethylene bridge; and additionally when A is CH then R³ and R⁵ may also be selected from fluoro, hydroxy or C₁₋₄alkoxy.

R³, R⁴, R⁵, R⁶, R⁷, R⁸, R⁹ and R¹⁰ independently represent H or a C₁₋₄alkyl group

R³, R⁴, R⁵, R⁶, R⁷, R⁸, R⁹ and R¹⁰ independently represent H.

R³ represents H or methyl and R⁴, R⁵, R⁶, R⁷, R⁸, R⁹ and R¹⁰ independently represent H.

R³ represents methyl and R⁴, R⁵, R⁶, R⁷, R⁸, R⁹ and R¹⁰ independently represent H.

The term “a 5-membered heteroaromatic group containing 1, 2, 3 or 4 hetero atoms selected from O, N and S” includes pyrrolyl, thienyl, furyl, pyrazolyl, imidazolyl, oxazolyl, isoxazolyl, thiazolyl, isothiazolyl, thiadiazolyl, 1,3,4-oxadiazolyl, 1,2,4-oxadiazolyl, triazolyl, furazanyl and tetrazolyl each of which is optionally substituted as previously stated. Particularly the group is 1,2,4-triazol-1-yl, 1,2,4-oxadiazol-3-yl, 1,2,4-oxadiazol-3-yl, 4-(tetrazol-1-yl) or pyrazol-1-yl. More particularly the group is 1,2,4-triazol-1-yl, 1,2,4-oxadiazol-3-yl, 1,2,4-oxadiazol-3-yl or 4-(tetrazol-1-yl),

The term “a carbon linked saturated 4 to 7 membered heterocyclic group containing one or more N, S or O, wherein the S may be in its oxidised form of SO or SO₂ includes oxetanyl, tetrahydrofuranyl, tetrahydropyranyl, imidazolidinyl,1,3-thiazolidinyl, 1,3-oxazolidinyl, oxepanyl, azetidinyl, pyrrolidinyl, morpholinyl, thiamorpholinyl (perhydro-1,4-thiazinyl), perhydroazepinyl, perhydrooxazepinyl, tetrahydro-1,4-thiazinyl, 1-oxotetrahydrothienyl, 1,1-dioxotetrahydro-1,4-thiazinyl, piperidinyl, homopiperidinyl, piperazinyl or homopiperazinyl each of which may be optionally substituted as previously described. In particular the group is pyrrolidinyl, piperidinyl, morpholinyl, piperazinyl, oxetanyl, tetrahydrofuranyl or tetrahydropyranyl. More particularly the group is oxetanyl, tetrahydrofuranyl or tetrahydropyranyl. Most particularly the group is pyrrolidinyl, piperidinyl, morpholinyl or piperazinyl.

When two substituents on a nitrogen atom together with the nitrogen atom to which they are attached represent a saturated 4-7 membered heterocyclic group optionally containing an additional N, S or O then such rings include azetidino, pyrrolidino, is morpholino, piperidino, imidazolidinyl, piperazino, thiamorpholino (perhydro-1,4-thiazinyl), homopiperazino, perhydroazepino, perhydrooxazepino, 1,3-thiazolidinyl, 1,3-oxazolidinyl, oxepanyl, oxazepanyl and homopiperidinyl, each of which is optionally substituted as previously described. Particularly the saturated 4-7 membered heterocyclic group is pyrrolidinyl, piperidinyl, morpholinyl or piperazinyl, each of which is optionally substituted as previously described.

In another aspect the present invention provides one or more compounds selected from:

### List 1

tert-butyl 4-(5-(4-(methylsulfonyl)benzyloxy)pyrimidin-2-yl)piperazine-1-carboxylate;
tert-butyl 4-(5-(4-(1H-1,2,4-triazol-1-yl)benzyloxy)pyrimidin-2-yl)piperazine-1-carboxylate;
tert-butyl 4-(5-(pyridin-4-ylmethoxy)pyrimidin-2-yl)piperazine-1-carboxylate;
tert-butyl 4-(5-(4-(5-methyl-1,2,4-oxadiazol-3-yl)benzyloxy)pyrimidin-2-yl)piperazine-1-carboxylate;
tert-butyl 4-(5-((6-(1H-pyrazol-1-yl)pyridin-3-yl)methoxy)pyrimidin-2-yl)piperazine-1-carboxylate;
tert-butyl 4-(5-((6-acetamidopyridin-3-yl)methoxy)pyrimidin-2-yl)piperazine-1-carboxylate;
tert-butyl 4-(5-(4-(1H-tetrazol-1-yl)benzyloxy)pyrimidin-2-yl)piperazine-1-carboxylate;
tert-butyl 4-(5-((5-fluoro-2-methoxypyridin-4-yl)methoxy)pyrimidin-2-yl)piperazine-1-carboxylate;
tert-butyl 4-(5-(pyrimidin-4-ylmethoxy)pyrimidin-2-yl)piperazine-1-carboxylate;
tert-butyl 4-(5-((6-(1H-1,2,4-triazol-1-yl)pyridin-3-yl)methoxy)pyrimidin-2-yl)piperazine-1-carboxylate;
tert-butyl 4-(5-((3-methoxypyridin-4-yl)methoxy)pyrimidin-2-yl)piperazine-1-carboxylate;
tert-butyl 4-(5-(4-isobutyramido-3-methylbenzyloxy)pyrimidin-2-yl)piperazine-1-carboxylate;
tert-butyl 4-(5-(3-methyl-4-pivalamidobenzyloxy)pyrimidin-2-yl)piperazine-1-carboxylate;
tert-butyl 4-(5-(4-isobutyramidobenzyloxy)pyrimidin-2-yl)piperazine-1-carboxylate;
tert-butyl 4-(5-(4-pivalamidobenzyloxy)pyrimidin-2-yl)piperazine-1-carboxylate;
tert-butyl 4-(5-(4-(N-methylsulfamoyl)benzyloxy)pyrimidin-2-yl)piperazine-1-carboxylate;
tert-butyl 4-(5-(4-(methylsulfonyloxy)benzyloxy)pyrimidin-2-yl)piperazine-1-carboxylate;
(R)-tert-butyl 3-methyl-4-(5-(4-(methylsulfonyl)benzyloxy)pyrimidin-2-yl)piperazine-1-carboxylate;
(R)-tert-butyl 3-methyl-4-(5-(pyridin-4-ylmethoxy)pyrimidin-2-yl)piperazine-1-carboxylate;
(R)-tert-butyl 4-(5-(4-(1H-tetrazol-1-yl)benzyloxy)pyrimidin-2-yl)-3-methylpiperazine-1-carboxylate;
(R)-tert-butyl 4-(5-(4-((2-hydroxyethyl)(methyl)carbamoyl)benzyloxy)pyrimidin-2-yl)-3-methylpiperazine-1-carboxylate;
(S)-tert-butyl 2-methyl-4-(5-(4-(methylsulfonyl)benzyloxy)pyrimidin-2-yl)piperazine-1-carboxylate;
(R)-tert-butyl 2-methyl-4-(5-(4-(methylsulfonyl)benzyloxy)pyrimidin-2-yl)piperazine-1-carboxylate;
(S)-tert-butyl 3-methyl-4-(5-(4-(methylsulfonyl)benzyloxy)pyrimidin-2-yl)piperazine-1-carboxylate;
(2R,5S)-tert-butyl 2,5-dimethyl-4-(5-(4-(methylsulfonyl)benzyloxy)pyrimidin-2-yl)piperazine-1-carboxylate;
(3R,5S)-tert-butyl 3,5-dimethyl-4-(5-(4-(methylsulfonyl)benzyloxy)pyrimidin-2-yl)piperazine-1-carboxylate;
tert-butyl 3,3-dimethyl-4-(5-(4-(methylsulfonyl)benzyloxy)pyrimidin-2-yl)piperazine-1-carboxylate;
(1R,4R)-tert-butyl 5-(5-(4-(methylsulfonyl)benzyloxy)pyrimidin-2-yl)-2,5-diazabicyclo[2.2.1]heptane-2-carboxylate;
(1S,4S)-tert-butyl 5-(5-(4-(methylsulfonyl)benzyloxy)pyrimidin-2-yl)-2,5-diazabicyclo[2.2.2]octane-2-carboxylate;
tert-butyl 4-(5-((6-(methylsulfonyl)pyridin-3-yl)methoxy)pyrimidin-2-yl)piperazine-1-carboxylate;
tert-butyl 4-(5-((5-(methylsulfonyl)pyridin-2-yl)methoxy)pyrimidin-2-yl)piperazine-1-carboxylate;
tert-butyl 4-(5-(3-fluoro-4-(methylsulfonyl)benzyloxy)pyrimidin-2-yl)piperazine-1-carboxylate;
tert-butyl 4-(5-(2-methyl-4-(methylsulfonyl)benzyloxy)pyrimidin-2-yl)piperazine-1-carboxylate;
(R)-tert-butyl 3-methyl-4-(5-((5-(methylsulfonyl)pyridin-2-yl)methoxy)pyrimidin-2-yl)piperazine-1-carboxylate;
(R)-tert-butyl 4-(5-(2-fluoro-4-(methylsulfonyl)benzyloxy)pyrimidin-2-yl)-3-methylpiperazine-1-carboxylate;
tert-butyl 4-(5-(2-fluoro-4-(methylsulfonyl)benzyloxy)pyrimidin-2-yl)piperazine-1-carboxylate;
tert-butyl 4-(5-(3-methyl-4-(methylsulfonyl)benzyloxy)pyrimidin-2-yl)piperazine-1-carboxylate;
tert-butyl 4-(5-((3-cyanopyridin-4-yl)methoxy)pyrimidin-2-yl)piperazine-1-carboxylate;
(R)-tert-butyl 4-(5-((3-cyanopyridin-4-yl)methoxy)pyrimidin-2-yl)-3-methylpiperazine-1-carboxylate;
4-((2-(4-(5-fluoropyrimidin-2-yl)piperazin-1-yl)pyrimidin-5-yloxy)methyl)nicotinonitrile;
(R)-4-((2-(4-(5-fluoropyrimidin-2-yl)-2-methylpiperazin-1-yl)pyrimidin-5-yloxy)methyl)nicotinonitrile;
5-fluoro-2-(4-(5-(4-(methylsulfonyl)benzyloxy)pyrimidin-2-yl)piperazin-1-yl)pyrimidine;
tert-butyl 4-(5-(4-((2-hydroxyethyl)(methyl)carbamoyl)benzyloxy)pyrimidin-2-yl)piperazine-1-carboxylate;
tert-butyl 4-(5-(4-(2-hydroxyethylcarbamoyl)benzyloxy)pyrimidin-2-yl)piperazine-1-carboxylate;
tert-butyl 4-(5-(4-(methylcarbamoyl)benzyloxy)pyrimidin-2-yl)piperazine-1-carboxylate;
tert-butyl 4-(5-(4-(isopropylcarbamoyl)benzyloxy)pyrimidin-2-yl)piperazine-1-carboxylate;
tert-butyl 4-(5-(4-(tert-butylcarbamoyl)benzyloxy)pyrimidin-2-yl)piperazine-1-carboxylate;
tert-butyl 4-(5-(4-(2-(dimethylamino)ethylcarbamoyl)benzyloxy)pyrimidin-2-yl)piperazine-1-carboxylate;
tert-butyl 4-(5-(4-((2-(dimethylamino)ethyl)(methyl)carbamoyl)benzyloxy)pyrimidin-2-yl)piperazine-1-carboxylate;
tert-butyl 4-(5-(4-(piperazine-1-carbonyl)benzyloxy)pyrimidin-2-yl)piperazine-1-carboxylate;
tert-butyl 4-(5-(4-(4-methylpiperazine-1-carbonyl)benzyloxy)pyrimidin-2-yl)piperazine-1-carboxylate;
tert-butyl 4-(5-(4-((1-methylpiperidin-4-yl)methylcarbamoyl)benzyloxy)pyrimidin-2-yl)piperazine-1-carboxylate;
tert-butyl 4-(5-(4-(morpholin-4-yl -1-carbonyl)benzyloxy)pyrimidin-2-yl)piperazine-1-carboxylate;
tert-butyl 4-(5-(4-(isopropylcarbamoyl)-3-methylbenzyloxy)pyrimidin-2-yl)piperazine-1-carboxylate;
tert-butyl 4-(5-(4-(tert-butylcarbamoyl)-3-methylbenzyloxy)pyrimidin-2-yl)piperazine-1-carboxylate;
isopropyl 4-(5-(4-(methylsulfonyl)benzyloxy)pyrimidin-2-yl)piperazine-1-carboxylate;
(R)-isopropyl 3-methyl-4-(5-(4-(methylsulfonyl)benzyloxy)pyrimidin-2-yl)piperazine-1-carboxylate;
isopropyl 4-(5-((3-cyanopyridin-4-yl)methoxy)pyrimidin-2-yl)piperazine-1-carboxylate;
(R)-isopropyl 4-(5-((3-cyanopyridin-4-yl)methoxy)pyrimidin-2-yl)-3-methylpiperazine-1-carboxylate;
1-methylcyclopropyl 4-(5-(4-(methylsulfonyl)benzyloxy)pyrimidin-2-yl)piperazine-1-carboxylate;
(R)-1-methylcyclopropyl 3-methyl-4-(5-(4-(methylsulfonyl)benzyloxy)pyrimidin-2-yl)piperazine-1-carboxylate;
1-methylcyclopropyl 4-(5-((3-cyanopyridin-4-yl)methoxy)pyrimidin-2-yl)piperazine-1-carboxylate;
cyclobutyl 4-(5-(4-(methylsulfonyl)benzyloxy)pyrimidin-2-yl)piperazine-1-carboxylate; (R)-cyclobutyl 3-methyl-4-(5-(4-(methylsulfonyl)benzyloxy)pyrimidin-2-yl)piperazine-1-carboxylate;
cyclobutyl 4-(5-((3-cyanopyridin-4-yl)methoxy)pyrimidin-2-yl)piperazine-1-carboxylate;
1,1,1-trifluoropropan-2-yl 4-(5-(4-(methylsulfonyl)benzyloxy)pyrimidin-2-yl)piperazine-1-carboxylate;
(3R)-1,1,1-trifluoropropan-2-yl 3-methyl-4-(5-(4-(methylsulfonyl)benzyloxy)pyrimidin-2-yl)piperazine-1-carboxylate;
1,1,1-trifluoropropan-2-yl 4-(5-((3-cyanopyridin-4-yl)methoxy)pyrimidin-2-yl)piperazine-1-carboxylate;
2-cyanopropan-2-yl 4-(5-(4-(methylsulfonyl)benzyloxy)pyrimidin-2-yl)piperazine-1-carboxylate;
(R)-2-cyanopropan-2-yl 3-methyl-4-(5-(4-(methylsulfonyl)benzyloxy)pyrimidin-2-yl)piperazine-1-carboxylate;
2-cyanopropan-2-yl 4-(5-((3-cyanopyridin-4-yl)methoxy)pyrimidin-2-yl)piperazine-1-carboxylate;
(R)-2-cyanopropan-2-yl 4-(5-((3-cyanopyridin-4-yl)methoxy)pyrimidin-2-yl)-3-methylpiperazine-1-carboxylate;
oxetan-3-yl 4-(5-(4-(methylsulfonyl)benzyloxy)pyrimidin-2-yl)piperazine-1-carboxylate;
oxetan-3-yl 4-(5-((3-cyanopyridin-4-yl)methoxy)pyrimidin-2-yl)piperazine-1-carboxylate;
(3R)-tetrahydrofuran-3-yl 3-methyl-4-(5-(4-(methylsulfonyl)benzyloxy)pyrimidin-2-yl)piperazine-1-carboxylate;
3-methyloxetan-3-yl 4-(5-(4-(methylsulfonyl)benzyloxy)pyrimidin-2-yl)piperazine-1-carboxylate;
(R)-3-methyloxetan-3-yl 3-methyl-4-(5-(4-(methylsulfonyl)benzyloxy)pyrimidin-2-yl)piperazine-1-carboxylate;
3-methyloxetan-3-yl 4-(5-((3-cyanopyridin-4-yl)methoxy)pyrimidin-2-yl)piperazine-1-carboxylate;
(R)-tetrahydro-2H-pyran-4-yl 4-(5-((3-cyanopyridin-4-yl)methoxy)pyrimidin-2-yl)-3-methylpiperazine-1-carboxylate;
tert-butyl 4-(5-(4-(ethylsulfonyl)benzyloxy)pyrimidin-2-yl)piperazine-1-carboxylate;
tert-butyl 4-(5-(4-(cyclopropylsulfonyl)benzyloxy)pyrimidin-2-yl)piperazine-1-carboxylate;
4-((2-(4-(5-isopropyl-1,2,4-oxadiazol-3-yl)piperazin-1-yl)pyrimidin-5-yloxy)methyl)nicotinonitrile;
(R)-4-((2-(4-(5-isopropyl-1,2,4-oxadiazol-3-yl)-2-methylpiperazin-1-yl)pyrimidin-5-yloxy)methyl)nicotinonitrile;
5-isopropyl-3-(4-(5-(4-(methylsulfonyl)benzyloxy)pyrimidin-2-yl)piperazin-1-yl)-1,2,4-oxadiazole;
3-(4-(5-(4-(methylsulfonyl)benzyloxy)pyrimidin-2-yl)piperazin-1-yl)-5-(trifluoromethyl)-1,2,4-oxadiazole;
(R)-5-isopropyl-3-(3-methyl-4-(5-(4-(methylsulfonyl)benzyloxy)pyrimidin-2-yl)piperazin-1-yl)-1,2,4-oxadiazole;
3-(4-(5-(4-(methylsulfonyl)benzyloxy)pyrimidin-2-yl)piperazin-1-yl)-5-(trifluoromethyl)-1,2,4-oxadiazole;
tert-butyl 4-(5-(4-(trifluoromethylsulfinyl)benzyloxy)pyrimidin-2-yl)piperazine-1-carboxylate;
tert-butyl 4-(5-(4-(2-morpholinoethylsulfonyl)benzyloxy)pyrimidin-2-yl)piperazine-1-carboxylate;
tert-butyl 4-(5-(4-(methylsulfinyl)benzyloxy)pyrimidin-2-yl)piperazine-1-carboxylate;
tert-butyl 4-(5-((2-isobutyramidopyrimidin-5-yl)methoxy)pyrimidin-2-yl)piperazine-1-carboxylate;
tert-butyl 4-(5-((3-methylpyridin-4-yl)methoxy)pyrimidin-2-yl)piperazine-1-carboxylate;
(R)-4-((2-(4-(5-(difluoromethyl)-1,2,4-oxadiazol-3-yl)-2-methylpiperazin-1-yl)pyrimidin-5-yloxy)methyl)nicotinonitrile;
(R)-4-((2-(4-(5-cyclopropyl-1,2,4-oxadiazol-3-yl)-2-methylpiperazin-1-yl)pyrimidin-5-yloxy)methyl)nicotinonitrile;
(R)-4-((2-(4-(3-isopropyl-1,2,4-oxadiazol-5-yl)-2-methylpiperazin-1-yl)pyrimidin-5-yloxy)methyl)nicotinonitrile;
(R)-4-((2-(2-methyl-4-(3-(trifluoromethyl)-1,2,4-oxadiazol-5-yl)piperazin-1-yl)pyrimidin-5-yloxy)methyl)nicotinonitrile;
4-((2-((R)-4-(5-((S)-1-methoxyethyl)-1,2,4-oxadiazol-3-yl)-2-methylpiperazin-1-yl)pyrimidin-5-yloxy)methyl)nicotinonitrile;4-((2-((R)-4-(5-((R)-1-methoxyethyl)-1,2,4-oxadiazol-3-yl)-2-methylpiperazin-1-yl)pyrimidin-5-yloxy)methyl)nicotinonitrile;
(R)-4-((2-(2-methyl-4-(5-(trifluoromethyl)-1,2,4-oxadiazol-3-yl)piperazin-1-yl)pyrimidin-5-yloxy)methyl)nicotinonitrile;
(R)-((R)-1,1,1-trifluoropropan-2-yl) 4-(5-((3-cyanopyridin-4-yl)methoxy)pyrimidin-2-yl)-3-methylpiperazine-1-carboxylate;
(R)-((S)-1,1,1-trifluoropropan-2-yl) 4-(5-((3-cyanopyridin-4-yl)methoxy)pyrimidin-2-yl)-3-methylpiperazine-1-carboxylate;
(R)-2,2,2-trifluoroethyl 4-(5-((3-cyanopyridin-4-yl)methoxy)pyrimidin-2-yl)-3-methylpiperazine-1-carboxylate;
2,2,2-trifluoroethyl 4-(5-((3-cyanopyridin-4-yl)methoxy)pyrimidin-2-yl)piperazine-1-carboxylate;
(R)-4-((2-(2-methyl-4-(5-(3-methyloxetan-3-yl)-1,2,4-oxadiazol-3-yl)piperazin-1-yl)pyrimidin-5-yloxy)methyl)nicotinonitrile;
(R)-3-(trifluoromethyl)oxetan-3-yl 4-(5-(2-fluoro-4-(methylsulfonylmethyl)benzyloxy)-pyrimidin-2-yl)-3-methylpiperazine-1-carboxylate;
(R)-3-(trifluoromethyl)oxetan-3-yl 4-(5-(2-fluoro-4-(methylsulfonyl)benzyloxy)pyrimidin-2-yl)-3-methylpiperazine-1-carboxylate;
(R)-3-(trifluoromethyl)oxetan-3-yl 3-methyl-4-(5-(4-(methylsulfonyl)benzyloxy)pyrimidin-2-yl)piperazine-1-carboxylate; and
(R)-3-(trifluoromethyl)oxetan-3-yl 4-(5-((3-cyanopyridin-4-yl)methoxy)pyrimidin-2-yl)-3-methylpiperazine-1-carboxylate;
   or a pharmaceutically acceptable salt thereof.

The present invention also provides one of the compounds from the above list or any number of the compounds in the above list from 1 to 112 inclusive. In another aspect the present invention provides a compound of formula I, II, III or IV as defined in any of the definitions above or in the appended claims but excluding any one or more of the compounds in the list of compounds immediately above.

In another aspect the present invention provides one of the compounds from the List 1 or any number of the compounds in the above list between 1 and 112 inclusive wherein the compounds are as listed and are not in the form of a salt.

In another aspect the present invention provides one or more of the following compounds:
(R)-4-((2-(4-(5-isopropyl-1,2,4-oxadiazol-3-yl)-2-methylpiperazin-1-yl)pyrimidin-5-yloxy)methyl)nicotinonitrile;
(R)-4-((2-(4-(3-isopropyl-1,2,4-oxadiazol-5-yl)-2-methylpiperazin-1-yl)pyrimidin-5-yloxy)methyl)nicotinonitrile;
(R)-4-((2-(2-methyl-4-(3-(trifluoromethyl)-1,2,4-oxadiazol-5-yl)piperazin-1-yl)pyrimidin-5-yloxy)methyl)nicotinonitrile;
4-((2-((R)-4-(5-((S)-1-methoxyethyl)-1,2,4-oxadiazol-3-yl)-2-methylpiperazin-1-yl)pyrimidin-5-yloxy)methyl)nicotinonitrile;
4-((2-((R)-4-(5-((R)-1-methoxyethyl)-1,2,4-oxadiazol-3-yl)-2-methylpiperazin-1-yl)pyrimidin-5-yloxy)methyl)nicotinonitrile;
(R)—((R)-1,1,1-trifluoropropan-2-yl) 4-(5-((3-cyanopyridin-4-yl)methoxy)pyrimidin-2-yl)-3-methylpiperazine-1-carboxylate;
(R)—((S)-1,1,1-trifluoropropan-2-yl) 4-(5-((3-cyanopyridin-4-yl)methoxy)pyrimidin-2-yl)-3-methylpiperazine-1-carboxylate;
(R)-3-(trifluoromethyl)oxetan-3-yl 4-(5-(2-fluoro-4-(methylsulfonylmethyl)benzyloxy)-pyrimidin-2-yl)-3-methylpiperazine-1-carboxylate;
(R)-3-(trifluoromethyl)oxetan-3-yl 4-(5-(2-fluoro-4-(methylsulfonyl)benzyloxy)pyrimidin-2-yl)-3-methylpiperazine-1-carboxylate; and
(R)-3-(trifluoromethyl)oxetan-3-yl 4-(5-((3-cyanopyridin-4-yl)methoxy)pyrimidin-2-yl)-3-methylpiperazine-1-carboxylate;
   or a pharmaceutically acceptable salt thereof.

The present invention also provides one of the compounds from the list immediately above or any number of the compounds in that list from 1 to 10 inclusive. In another aspect the present invention provides one of the compounds from the list immediately above or any number of the compounds in that list between 1 and 10 wherein the compounds are as listed and are not in the form of a salt. In another aspect the present invention provides a compound of formula I, II, III or IV as defined in any of the definitions above or in the appended claims but excluding any one or more of the compounds in the list of compounds immediately above.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl, t-butyl, pentyl, isopentyl, neopentyl, tert-pentyl, hexyl and isohexyl. Particular alkyl groups are methyl, ethyl, propyl, isopropyl, butyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, is chlorine, bromine or iodine. Particularly the term “halogen” means fluorine, chlorine, or bromine

Examples of “C₁₋₆alkanoyl” include C₁₋₄alkanoyl, propionyl and acetyl. Examples of “C₁₋₄alkylsulfonyl” include methanesulfonyl and ethanesulfonyl. Examples of “C₁₋₆alkylsulfonyloxy” include C₁₋₄alkylsulfonyloxy, methanesulfonyloxy and ethanesulfonyloxy. Examples of “C₁₋₆alkoxycarbonyl” include C₁₋₄alkoxycarbonyl, methoxycarbonyl, ethoxycarbonyl, n- and t-butoxycarbonyl. Examples of “C₁₋₆alkoxy” include methoxy, ethoxy and propoxy. Examples of “C₂₋₄alkanoylamino” include acetamido and propionylamino. Examples of “C₁₋₆alkylthio” include C₁₋₄alkylthio, methylthio and ethylthio. Examples of “C₁₋₆alkylsulfinyl” include C₁₋₄alkylsulfinyl, methylsulfinyl and ethylsulfinyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂carbamoyl” are N,N—(C₁₋₄alkyl)carbamoyl, dimethylaminocarbonyl and methylethylaminocarbonyl. Examples of C₃₋₆cycloalkyl include cyclopropyl, cyclobutyl, cyclopentyl and cyclohexyl. Examples of C₃₋₆cycloalkoxy include C₃₋₄cycloalkoxy cyclopropyloxy, cyclobutyloxy, cyclopentyloxy and cyclohexyloxy.

“Pharmaceutically acceptable salt”, where such salts are possible, includes both pharmaceutically acceptable acid and base addition salts. A suitable pharmaceutically acceptable salt of a compound of formula I is, for example, an acid-addition salt of a compound of formula I which is sufficiently basic, for example an acid-addition salt with an inorganic or organic acid such as hydrochloric, hydrobromic, sulphuric, trifluoroacetic, citric or maleic acid; or, for example a base-addition salt of a compound of formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, and diastereomers where such isomers, enantiomers and diastereomers exist, as well as pharmaceutically acceptable is salts thereof and solvates thereof such as for instance hydrates including solvates of the free compounds or solvates of a salt of the compound. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions that will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C, ¹⁹F, deuterium or tritium and their use as isotopically labelled compounds for pharmacological and metabolic studies.

The present invention also encompasses prodrugs of a compound of formula I that is compounds which are converted into a compound of formula I in vivo.

A compound of the Formula I, or a pharmaceutically-acceptable salt thereof, may be prepared by any process known to be applicable to the preparation of chemically-related compounds. Such processes, when used to prepare a compound of the formula I are provided as a further feature of the invention and are illustrated by the following representative process variants. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated that are within the ordinary skill of an organic chemist.

Another aspect of the present invention provides a process for preparing a compound of formula I or a pharmaceutically acceptable salt thereof which comprises any one of the following processes. In schemes 1, 2, 3 and 6, R³, R⁴, R⁵, R⁶, R⁷, R⁸, R⁹ and R¹⁰ have been omitted for clarity but it should be understood that these groups are present in structures 2 to 15 inclusive and may represent any of the substituents listed for them previously.

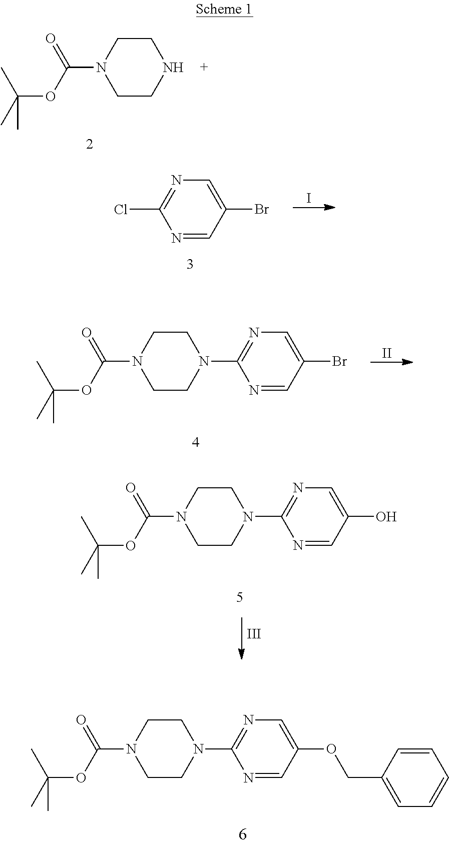

According to this scheme, a difunctionalised pyrimidine (2-Cl, 5-Br shown as an illustrative example) is reacted with a suitably protected amine (N-Boc piperazine shown as an illustrative example) to afford the functionalised pyrimidine 4 (Reaction I). Functional group interconversion to a phenol 5 (Reaction II) is followed by ether formation to give compound of formula 6 (Reaction III). Details of suitable methods for carrying out these transformations are given below:

Reaction I: Methods for the displacement of leaving groups at the 2-position of pyrimidines are well known in the art and examples are described in the following references; Tetrahedron Lett., 2007, 48(17), 3043; Tetrahedron Lett., 2006, 47(15), 2549; Tetrahedron Lett., 2002, 43(33), 5739. Reaction typically involves treatment with a base (e.g. cesium carbonate, potassium carbonate) in a solvent such as DMF or acetonitrile at a temperature from 25° C. to 80° C., and particularly at 25° C.

### Reaction II:

Methods for the conversion of halogens to hydroxy groups at the 5-position of pyrimidines are well known in the art and examples are described in the following references; J. Org. Chem., 2008, 73(23), 9326; Tetrahedron Lett., 2006, 47(41), 7363. One method involves formation of a boronic ester, typically by treatment with a source of boron (e.g. bis(pinacolato)diboron) with a suitable catalyst (e.g. Pd(OAc)₂) in a suitable solvent (e.g. DMF) at temperatures from 25° C. to 100° C. The intermediate may be isolated, or alternatively treated directly with a suitable oxidising agent (e.g. NaBO₃) to furnish the desired phenol. An alternative approach involves treatment of the halo compound with a suitable organometallic reagent (e.g. BuLi) in a suitable solvent (e.g. THF) followed by quenching of the intermediate metal species with a suitable boron species (e.g. triisopropyl borate) at low temperatures (typically −60 to −20° C.) followed by treatment with a suitable oxidising agent (e.g. hydrogen peroxide)

### Reaction III:

Methods for the conversion of phenols to ethers are well known in the art and an example is described in the following reference; J. Mat. Chem., 2008, 18(43), 5301. The process may be carried out using a displacement of a compound containing a suitable leaving group (e.g. halide, mesylate, tosylate) in the presence of a suitable base (e.g. cesium carbonate, potassium carbonate) in a suitable solvent such as DMF or acetonitrile typically at temperatures of between ambient and 100° C. and particularly at 25° C.

Alternatively the conversion can be carried out by Mitsunobu type chemistry as described in the following reference; Chem. Rev., 2009, 109(6), 2551. Typically reactions are carried out by treatment with triphenyl phosphine and diethyl azodicarboxylate or di-isopropyl azodicarboxylate in an inert solvent such as tetrahydrofuran, toluene or hexanes at a temperature from 25° C. to 80° C., and particularly at 25° C.

Further examples may be synthesised by removal of the carbamate group and further derivatisation as shown in Scheme 2. According to this scheme, the carbamate may be removed to produce an amine (7) (Reaction IV)

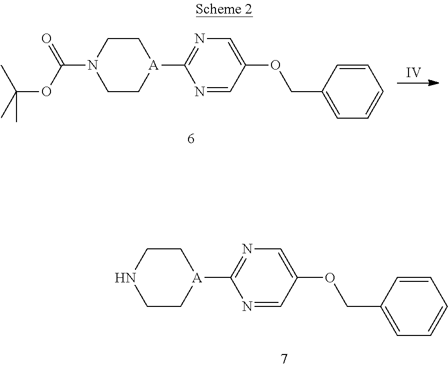

### Reaction IV:

Methods for the removal of carbamates are well known in the literature (for illustration see T. W. Green, Protective Groups in Organic Synthesis, John Wiley and Sons, 1991). The process may be carried out by treatment with a suitable acid (e.g. HCl, TFA) in a suitable solvent such as DCM or THF typically at ambient temperatures.

The amine may then be converted to a range of derivatives (e.g. sulphonamides, sulphonyl ureas, amides, N-linked heterocycles) using chemistry well known to the art (Reaction V)

A typical example of functionalisation with a heterocycle is shown in Scheme 3 in which R is H or C₁₋₄alkyl.

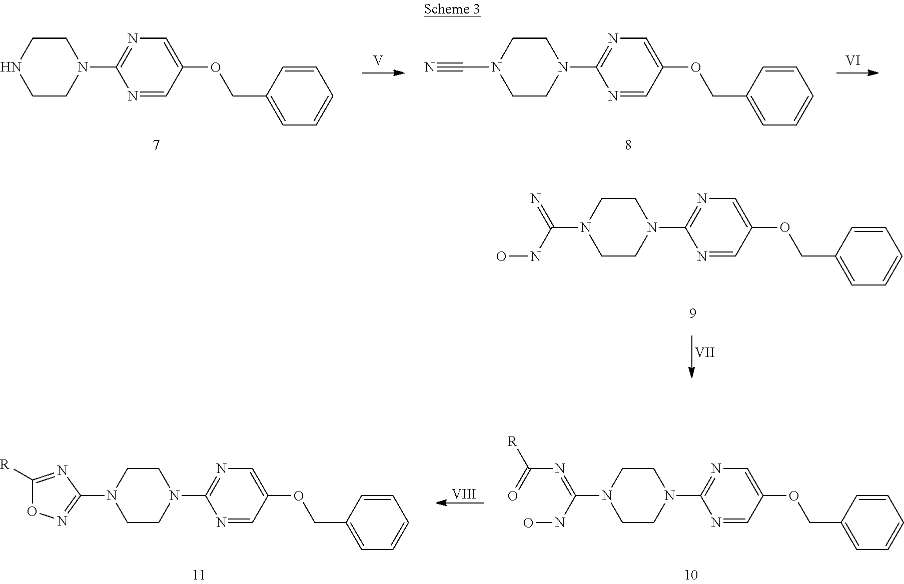

Reaction V-VIII: Methods for the conversion of amines to oxadiazoles are well known in the art and an example is described in the following reference; Bioorg. Med. Chem., 2008, 16(4), 1613.

This process may be carried out by functionalising an amine with cyanogen bromide in the presence of a suitable base (e.g. triethylamine) and solvent (e.g. DCM) at a temperature from 0° C. to ambient. This is then reacted with hydroxyalamine in the presence of a suitable base (e.g. potassium carbonate) in a suitable solvent (e.g. ethanol/water) at a temperature from 60° C. to 100° C. and then further functionalised by treatment with a carboxylic acid in the presence of a suitable coupling agent (e.g. 1-hydroxybenzotriazole) and base (e.g. N-ethyldiisopropylamine) in a suitable solvent (e.g. DMF) typically at ambient temperature. Ring closure to give the heterocycle can be carried out by heating at elevated temperature (typically 100-150° C.) in a suitable solvent (e.g. toluene)

Alternative carbamates may be synthesised by treatment of the amine with a range of carbamate forming reagents including (but not limited to chloroformates, N-succinimido carbamates, phenol carbamates, p-fluorophenol carbamates & p-nitrophenol carbamates). These processes are well known to the art and examples are described in the following reference; Current Org. Synth., 2007, 4(3), 308.

Functional group manipulation may be used to elaborate final compounds or produced functionalised building blocks for incorporation. Typical examples are shown below.

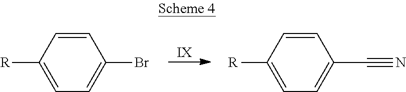

Methods for the conversion of aryl and heteroaryl halides to cyano groups are well known in the art and examples are described in the following references; Synthesis, 2008, 20, is 3351; Chem. Eur. 1, 2007, 13(21), 6249; Synlett, 2007, 4, 555; Tetrahedron Lett., 2006, 47(19), 3303; Tetrahedron Lett., 2005, 46(15), 2585; Tetrahedron Lett., 2004, 45(7), 1441. The process may be carried out using a displacement of a compound containing a suitable leaving group (e.g. bromide, iodide) in the presence of a suitable catalyst (e.g. tris(dibenzylideneacetone)dipalladium(0)) and a suitable ligand (e.g. xantphos) in a suitable solvent such as DMF typically at temperatures of between 100-150° C. optionally using microwave irradiation.

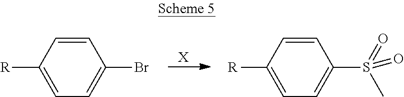

Methods for the conversion of aryl and heteroaryl halides to cyano groups are well known in the art and examples are described in the following references; J. Org. Chem., 2005, 70(7), 2696; Org. Lett., 2002, 4(25), 4423; Tetrahedron Lett., 2002, 43(47), 8479. The process may be carried out using a displacement of a compound containing a suitable leaving group (e.g. bromide, iodide) in the presence of a suitable catalyst (e.g. copper (1) trifluoromethanesulfonate toluene complex, copper (I) iodide) and a suitable ligand (e.g. L-proline) in a suitable solvent such as DMSO typically at temperatures of between 70-150° C. optionally using microwave irradiation.

When A=CH, compounds may be prepared as shown in Scheme 6.

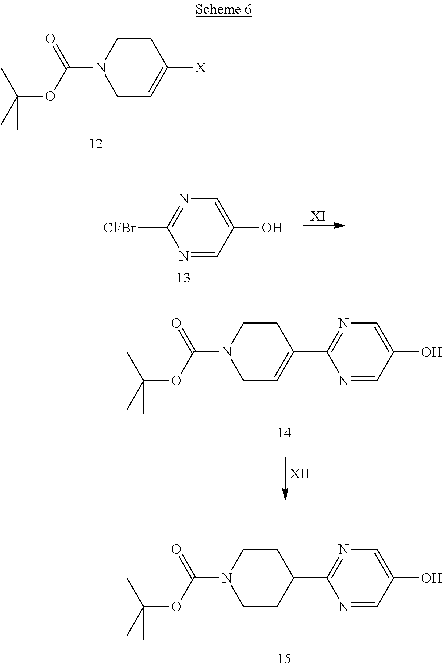

Reaction XI-XII: Methods for the reaction of functionalised tetrahydropyridines with halopyrimidines are well known in the art and an example is described in the following reference; Bioorg. Med. Chem. Lett., 2007, 17(23), 6539.

This process may be carried out by reacting an appropriate tetrahydropyridine (e.g. X=boronic acid or ester, typically pinacolate) with a 2-halo pyrimidine (e.g. bromo or chloro) in the presence of a suitable catalyst (e.g. dichloro 1,1′-bis(diphenylphosphino)ferrocene palladium(II)) and base (e.g. potassium carbonate) in a suitable solvent system (e.g. DME/water) typically at temperatures of between 50-150° C. and typically at 80 to 90° C. e.g. 85° C. optionally using microwave irradiation.

The resultant double bond may be saturated by treatment with hydrogen gas in the presence of a suitable catalyst (e.g. palladium, 10% on charcoal) in a suitable solvent (e.g. EtOH) at a temperature from 0° C. to ambient, typically at 20° C. Other hydrogenation techniques known to the art may also be used. Compounds of structure 15 may be converted in compounds of formula I using methods analogous to those used to convert compounds of structure 5 into compounds of formula I.

Formation of an amide from a carboxylic acid is a process well known to the art. Typical processes include, but are not limited to, formation of an acyl halide by treatment of the acid with a suitable reagent (e.g. oxalyl chloride, POCl₃) in a suitable solvent such as dichloromethane or N,N-dimethylformamide for example at temperatures ranging from 0-50° C. but particularly at ambient temperature. Alternatively, in situ conversion of the acid to an active ester derivative may be utilised with the addition of a suitable coupling agent (or combination of agents) such as HATU, HOBT, and EDAC for example, to form an active ester optionally in the presence of a suitable base such as triethylamine or N,N-di-iso-propylamine for example. Typically the reaction is carried out at temperatures ranging from 0-50° C. but particularly at ambient temperature.

Direct conversions of esters to amides are known in the art with examples described in the following references; J. Med. Chem., 2007, 50, 1675; Heterocycles, 2006, 67, 519 and typically involve heating of the two components, optionally in the presence of a suitable additive (e.g. Al(CH₃)₃). Typically reactions are carried out in inert solvents (e.g. toluene, benzene) at elevated temperatures (e.g. 50-150° C.) achieved through conventional or microwave heating.

It will be appreciated that certain of the various substituents in the compounds of the present invention may be introduced by standard aromatic substitution reactions or generated by conventional functional group modifications either prior to or immediately following the processes mentioned above, and as such are included in the process aspect of the invention. Such reactions and modifications include, introduction of a substituent by means of an aromatic substitution reaction, reduction of substituents, oxidation of substituents and alkylation of substituents, for example, alkylation reactions such as conversion of a secondary amide to a primary amide typically carried out using strong base (e.g. sodium hydride or lithium or potassium hexamethyldisilylazides) and a suitable alkylating agent (e.g. methyl iodide). The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (e.g. aluminium trichloride) under Friedel Crafts conditions; the introduction of an alkyl group using an alkyl halide and Lewis acid (e.g. aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl; removal of alkylthio groups by reductive de-sulphurisation by for example treatment with a nickel catalyst.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein. A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example hydroxylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

In one aspect the present invention provides a process for the preparation of a compound of formula I which comprises reacting a compound of formula V

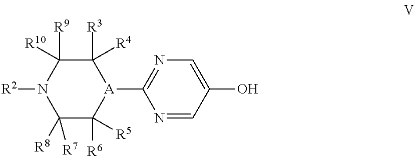

R², R³, R⁴, R⁵, R⁶, R⁷, R⁸, R⁹ and R¹⁰ are as previously defined with a compound of formula VI

R¹—CH₂OH VI

in which R¹ is as previously defined in the presence of a coupling agent for example is triphenyl phosphine and diethyl azodicarboxylate or di-isopropyl azodicarboxylate in an inert solvent such as tetrahydrofuran, toluene or hexanes at a temperature in the range of 0° C. to 80° C. and particularly in the range of 15° C. to 30° C.

In one aspect the present invention provides a process for the preparation of a compound of formula I which comprises reacting a compound of formula V

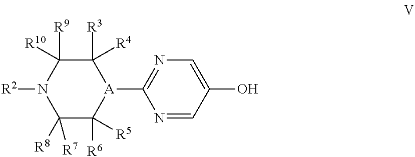

R², R³, R⁴, R⁵, R⁶, R⁷, R⁸, R⁹ and R¹⁰ are as previously defined with a compound of formula VII

R¹—CH₂—X VII

in which R¹ is as previously defined and X is a leaving group (e.g. halo, mesyloxy, tosyloxy) in the presence of a suitable base (e.g. cesium carbonate, potassium carbonate) in a suitable solvent such as DMF or acetonitrile typically at a temperature in the range of between 0 and 100° C. and particularly at a temperature in the range of 15° C. to 30° C.

In a further aspect the present invention provides a process for the preparation of a compound of formula I in which R² represents a group —C(O)OR^{x} in which R^{x} is previously defined which comprises reacting a compound of formula VIII

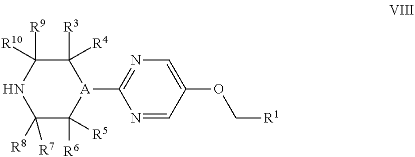

in which R¹, R³, R⁴, R⁵, R⁶, R⁷, R⁸, R⁹ and R¹⁰ are as previously defined with a carbonate of formula IX

R^{x}—O—CO—O—R^{y} IX

in which R^{x} is as previously defined and —O—R^{y} is a leaving group, for example phenyl or perfluorophenyl, for example if R^{y} is phenyl then the leaving group is phenoxy, in the presence of a suitable base (e.g. triethylamine) in the presence of an alcohol R^{x}—OH wherein R^{x} is the same as R^{x} in IX optionally in the presence of a solvent, for example chloroform, typically at a temperature in the range of between 0 and 150° C. and particularly at a temperature in the range of 50° C. to 100° C.

In a further aspect the present invention provides a process for the preparation of a compound of formula I in which R¹ represents 3-cyanopyridin-4-yl comprising reacting a compound of formula IX

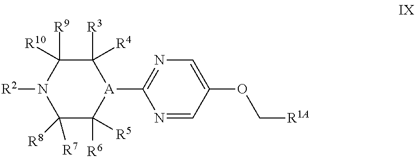

in which R², R³, R⁴, R⁵, R⁶, R⁷, R⁸, R⁹ and R¹⁰ are as previously defined and R^{1A} represents 3-bromopyridin4-yl or 3-iodoopyridin4-yl with a cyanide for example zinc cyanide in the presence of a suitable catalyst (e.g. tris(dibenzylideneacetone)dipalladium(0)) and a suitable ligand (e.g. xantphos) in a suitable solvent such as DMF typically at temperatures in the range of 30-150° C. for example in the range of 40-80° C. optionally using microwave irradiation.

Compounds of formula I in which R² represents a group —C(O)OR^{x} in which R^{x} is previously defined may be prepared by transesterifying compounds of formula I in which R² represents a group —C(O)OR^{x1} in which R^{x1} is a different R^{x} as previously defined.

It is believed that the intermediates disclosed herein are novel and are claimed herein as another aspect of the present invention. In particular are claimed compounds of formulae 4, 5, 7, 9, 10, 14 and 15.

### Pharmaceutical Compositions

A further feature of the invention is a pharmaceutical composition comprising a compound of Formula (I) as defined above, or a pharmaceutically-acceptable salt thereof, together with a pharmaceutically-acceptable diluent or carrier.

According to another aspect of the invention there is provided a compound of Formula (I) as defined above or a pharmaceutically-acceptable salt thereof for use as a medicament.

According to another aspect of the invention there is provided a compound of Formula (I), or a pharmaceutically-acceptable salt thereof as defined above for use as a medicament for treatment of a disease mediated through GPR119, in particular type 2 diabetes.

According to another aspect of the invention there is provided a compound of Formula (I), or a pharmaceutically-acceptable salt thereof as defined above for use in the treatment of a disease mediated through GPR119, in particular type 2 diabetes.

Further according to the invention there is provided the use of a compound of Formula (I) or a pharmaceutically-acceptable salt thereof in the preparation of a medicament for treatment of a disease mediated through GPR119, in particular type 2 diabetes.

The compound is suitably formulated as a pharmaceutical composition for use in this way.

According to another aspect of the present invention there is provided a method of treating GPR119 mediated diseases, especially diabetes, by administering an effective amount of a compound of Formula (I) or a pharmaceutically-acceptable salt thereof, to a mammal in need of such treatment.

Specific diseases which may be treated by a compound or composition of the invention include: blood glucose lowering in Type 2 Diabetes Mellitus without a serious risk of hypoglycaemia, dyslipidemia, obesity, insulin resistance, metabolic syndrome, syndrome X and impaired glucose tolerance.

Compounds of formula I are also expected to prevent or delay the development of type 2 diabetes from the metabolic syndrome and diabetes of pregnancy. Therefore the development of long-term complications associated with chronic hyperglycaemia in diabetes mellitus, such as the micro-angiopathies causing renal disease, retinal damage and peripheral vascular disease of the lower limbs including diabetic nephropathy, diabetic retinopathy and diabetic neuropathy, is expected to be delayed.

Compounds of formula I are also expected to prevent or delay the development of cardiovascular disease for example hypertension and atherosclerosis.

Compounds of formula I, or a pharmaceutically acceptable salt thereof, may also be useful in the treatment or prophylaxis of hepatic steatosis (including NASH), or fatty liver.

Compounds of formula I, or a pharmaceutically acceptable salt thereof, may also be useful in the treatment or prophylaxis of conditions related to low bone mass for example osteoporosis or may be useful in promoting an increase in bone mass.

According to another aspect of the invention there is provided the use of a compound of Formula (I) or a pharmaceutically-acceptable salt thereof, in the preparation of a medicament for use in the combined treatment or prevention, particularly treatment, of diabetes and obesity.

According to another aspect of the invention there is provided the use of a compound of Formula (I) or a pharmaceutically-acceptable salt thereof, in the preparation of a medicament for use in the treatment or prevention of obesity.

According to a further aspect of the invention there is provided a method for the combined treatment of obesity and diabetes by administering an effective amount of a compound of Formula (I) or a pharmaceutically-acceptable salt thereof, to a mammal in need of such treatment.

According to another aspect of the invention there is provided a compound of Formula (I) or a pharmaceutically-acceptable salt thereof as defined above for use as a medicament for treatment or prevention, particularly treatment of obesity.

According to a further aspect of the invention there is provided a method for the treatment of obesity by administering an effective amount of a compound of Formula (I) or a pharmaceutically-acceptable salt thereof, to a mammal in need of such treatment.

Compounds of the invention may be particularly suitable for use as pharmaceuticals, for example because of favourable physical and/or pharmacokinetic properties and/or toxicity profile.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). Dosage forms suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula (I) will naturally vary according to the nature and severity of the conditions, the age and sex of the animal or patient and the route of administration, according to well known principles of medicine.

In using a compound of the Formula (I) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

### Combinations

A compound of the invention may be used as the sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example in the treatment of diabetes mellitus, chemotherapy may include the following main categories of treatment:
1) Insulin and insulin analogues;
2) Insulin secretagogues including sulfonylureas (for example glibenclamide, glipizide), prandial glucose regulators (for example meglitindes e.g. repaglinide and nateglinide);
3) Dipeptidyl peptidase IV inhibitors (for example saxagliptin, sitagliptin, aloglitptin or vildagliptin);
4) Insulin sensitising agents including PPARgamma agonists (for example pioglitazone and rosiglitazone), and agents with combined PPARalpha and gamma activity;
5) Agents that modulate hepatic glucose balance (for example biguanides e.g. metformin, fructose 1, 6 bisphosphatase inhibitors, glycogen phopsphorylase inhibitors, glycogen synthase kinase inhibitors);
6) Agents designed to reduce the absorption of glucose from the intestine (for example alpha glucosidase inhibitors e.g. acarbose);
7) Agents that prevent the reabsorption of glucose by the kidney (for example SGLT-2 inhibitors for example dapagliflozin);
8) Agents designed to treat the complications of prolonged hyperglycaemia (for example aldose reductase inhibitors);
9) Anti-obesity agents (for example sibutramine and orlistat);
10) Anti-dyslipidaemia agents such as, HMG-CoA reductase inhibitors (eg statins for example rosuvastatin); PPARα agonists (fibrates, e.g. fenofibrate, clofibrate and gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
11) Antihypertensive agents such as, β blockers (eg atenolol, inderal); ACE inhibitors (eg lisinopril); Calcium antagonists (eg. nifedipine); Angiotensin receptor antagonists (eg candesartan), a antagonists and diuretic agents (eg. furosemide, benzthiazide);
12) Haemostasis modulators such as, antithrombotics, activators of fibrinolysis; thrombin antagonists; factor Xa inhibitors; factor VIIa inhibitors; antiplatelet agents (eg. aspirin, clopidogrel); anticoagulants (heparin and Low molecular weight analogues, hirudin) and warfarin;
13) Agents which antagonise the actions of glucagon;
14) Anti-inflammatory agents, such as non-steroidal anti-inflammatory drugs (eg.

aspirin) and steroidal anti-inflammatory agents (eg. cortisone); and
15) a glucokinase modulator
16) a ghrelin antibody;
17) a ghrelin antagonist;
18) an 11β HSD-1 inhibitor;
19) an UCP-1, 2 or 3 activator;
20) a CB1 receptor modulator for example an inverse agonist or an antagonist e.g. rimonabant or taranabant;
21) a melanin concentrating hormone (MCH) modulator for example an MCH-1 antagonist;
22) an NPY receptor modulator; for example an NPY agonist or an NPY2 agonist or an NPY5 antagonist;
23) an MC4r modulator for example an MC4r agonist;
24) an MC3r modulator for example an MC3r agonist;
25) an orexin receptor modulator for example an antagonist;
26) modulators of nuclear receptors for example LXR, FXR, RXR, GR, ERRα, β, PPARα, β, γ, δ and RORalpha;
27) a DGAT1 inhibitor;
28) a DGAT2 inhibitor;
29) a DGAT2 anti-sense oligonucleotide;
30) a fatty acid synthase inhibitor

### Cyclic AMP Assay for Human GPR119.

When an agonist binds to the GPR119 receptor adenylate cyclase is activated via Gₛ and the level of cAMP in cells increase. The amount of cAMP can be measured using a competitive immunoassay where native cAMP produced by the cells competes with cAMP labeled with the dye d2 (Cisbio, HTRF cAMP). A cryptate labeled anti-cAMP monoclonal antibody (Mab) visualizes the tracer binding by a principle based on HTRF technology (Homogenous Time-Resolved Fluorescence). The specific signal is inversely proportional to the concentration of native cAMP in the sample.

The ability of the compounds of the invention to activate GPR119 was demonstrated using the following in vitro human GPR119cyclic AMP (cAMP) assay by method A.

### Method A

The test compounds were dissolved in DMSO and added to a black 384-well low volume plate (Matrix) in a volume of 0.1 μl at a top concentration of 3 mM (corresponding to a concentration of 30 μM in the final assay). HEK 293s cells over-expressing human GPR119 (stored at −180° C.) were thawed and re-suspended in 37° C. growth media supplemented with 10% fetal calf serum, centrifuged and then re-suspended in assay buffer (20 mM HEPES pH 7.4, Hank's Balanced Salt Solution, 0.01% BSA, 1 mM IBMX). Cells were added to the assay plates at 2×10³ cells/well. Following a 45 minute incubate at room temperature, 5 μl of cAMP-d2 conjugate followed by 5 μl of anti cAMP-cryptate conjugate in lysis buffer was added to each well to lyse the cells and stop the cAMP production. Following a one hour incubation at room temperature the plate was read on a Envision plate reader using 320 nm excitation and 615 and 665 nm emission filters. The is fluorescence ratio (665 nm/615 nm×10⁴) was then determined for all data. The concentration of cAMP in each well was then determined by plotting the data on a standard curve. This was then used to determine the effects of test compounds on GPR119. The percent effect compared to the maximum (50 μM Oleoylethanolamide) and minimum (1% DMSO) assay controls was determined. The concentration and the percent effect of the test compound was fitted using a sigmoidal concentration-response model where EC₅₀ was determined as the concentration of the test compound at the midpoint of the dose response curve. Results are presented as percent effect at top concentration (30 μM) for exemplified compounds in the invention.

The results are given in the Table below.

Method A

| Ex | % activation @ 30 μM |
|---|---|
| 1 | 83 |
| 2 | 131 |
| 3 | 71 |
| 4 | 69 |
| 5 | 82 |
| 6 | 59 |
| 7 | 170 |
| 8 | 26 |
| 9 | 61 |
| 10 | 88 |
| 11 | 22 |
| 12 | 54 |
| 13 | 41 |
| 14 | 78 |
| 15 | 80 |
| 16 | 57 |
| 17 | 65 |
| 18 | 155 |
| 19 | 68 |
| 20 | 89 |
| 21 | 100 |
| 22 | 163 |
| 23 | 106 |
| 24 | 63 |
| 25 | 152 |
| 26 | 104 |
| 27 | 41 |
| 28 | 15 |
| 29 | 36 |
| 30 | 86 |
| 31 | 86 |
| 32 | 62 |
| 33 | 82 |
| 34 | 68 |
| 35 | 149 |
| 36 | 128 |
| 37 | 52 |
| 38 | 155 |
| 39 | 171 |
| 40 | 90 |
| 41 | 104 |
| 42 | 64 |
| 43 | 48 |
| 44 | 61 |
| 45 | 142 |
| 46 | 94 |
| 47 | 109 |
| 48 | 26 |
| 49 | 27 |
| 50 | 23 |
| 51 | 42 |
| 52 | 20 |
| 53 | 44 |
| 54 | 51 |
| 55 | 65 |
| 56 | 63 |
| 57 | 69 |
| 58 | 154 |
| 59 | 171 |
| 60 | 90 |
| 61 | 91 |
| 62 | 208 |
| 63 | 52 |
| 64 | 57 |
| 65 | 164 |
| 66 | 56 |
| 67 | 60 |
| 68 | 169 |
| 70 | 28 |
| 71 | 73 |
| 72 | 173 |
| 73 | 125 |
| 74 | 53 |
| 75 | 31 |
| 76 | 30 |
| 77 | 92 |
| 78 | 8 |
| 79 | 37 |
| 80 | 41 |
| 81 | 143 |
| 82 | 102 |
| 83 | 66 |
| 84 | 48 |
| 85 | 167 |
| 86 | 275 |
| 87 | 48 |
| 88 | 79 |
| 89 | 73 |
| 90 | 75 |
| 91 | 32 |
| 92 | 68 |
| 93 | 82 |
| 94 | 44 |
| 95 | 101 |
| 96 | 240 |
| 97 | 161 |
| 98 | 199 |
| 99 | 236 |
| 100 | 212 |
| 101 | 252 |
| 102 | 243 |
| 103 | 244 |
| 104 | 315 |
| 105 | 132 |
| 106 | 193 |
| 107 | 188 |
| 108 | 88 |
| 109 | 106 |
| 110 | 55 |
| 111 | 171 |
| 112 | 275 |

EC50 values (for selected examples)

| Ex | EC50 (μM) |
|---|---|
| 10 | 0.103 |
| 17 | 0.02 |
| 18 | 0.02 |
| 20 | 0.006 |
| 21 | 0.202 |
| 25 | 0.039 |
| 35 | 0.003 |
| 36 | 0.019 |
| 37 | 0.02 |
| 38 | 0.021 |
| 42 | 0.156 |
| 46 | 0.032 |
| 49 | 2.041 |
| 62 | 0.116 |
| 67 | 0.039 |
| 73 | 0.054 |
| 82 | 0.675 |
| 83 | 0.123 |
| 86 | 0.008 |
| 90 | 0.026 |
| 96 | 0.021 |
| 97 | 0.014 |
| 98 | 0.009 |
| 99 | 0.005 |
| 108 | 0.027 |

The following compounds did not show significant activity at a top concentration of 30 μM:
tert-butyl 4-[5-[[4-[(1-methyl-4-piperidyl)carbamoyl]phenyl]methoxy]pyrimidin-2-yl]piperazine-1-carboxylate;
tert-butyl 4-[5-[(2,6-dimethoxy-4-pyridyl)methoxy]pyrimidin-2-yl]piperazine-1-carboxylate;
tert-butyl 4-[5-[(2-acetamidopyrimidin-5-yl)methoxy]pyrimidin-2-yl]piperazine-1-carboxylate;
tetrahydrofuran-3-yl 4-[5-[(4-methylsulfonylphenyl)methoxy]pyrimidin-2-yl]piperazine-1-carboxylate;
tetrahydropyran-4-yl 4-[5-[3-cyano-4-pyridyl)methoxy]pyrimidin-2-yl]piperazine-1-carboxylate;
tert-butyl 4-[5-[[4-[methyl-(1-methyl-4-piperidyl)carbamoyl]phenyl]methoxy]pyrimidin-2-yl]piperazine-1-carboxylate;
tert-butyl 4-[5-[(2-methyl-4-pyridyl)methoxy]pyrimidin-2-yl]piperazine-1-carboxylate;
tetrahydropyran-4-yl (3R)-3-methyl-4-[5-[(4-methylsulfonylphenyl)methoxy]pyrimidin-2-yl]piperazine-1-carboxylate;
tetrahydropyran-4-yl 4-[5-[(4-methylsulfonylphenyl)methoxy]pyrimidin-2-yl]piperazine-1-carboxylate;
tetrahydrofuran-3-yl 4-[5-[(3-cyano-4-pyridyl)methoxy]pyrimidin-2-yl]piperazine-1-carboxylate;

4-[[2-[4-(5-fluoropyrimidin-2-yl)piperazin-1-yl]pyrimidin-5-yl]oxymethyl]-N-(2-hydroxyethyl)-N-methyl-benzamide;
tert-butyl 4-[5-[(3-methylsulfonyl-4-pyridyl)methoxy]pyrimidin-2-yl]piperazine-1-carboxylate;
tert-butyl 4-[5-[[3-(methylcarbamoyl)-4-pyridyl]methoxy]pyrimidin-2-yl]piperazine-1-carboxylate;
tert-butyl 4-[5-[[3-(dimethylcarbamoyl)-4-pyridyl]methoxy]pyrimidin-2-yl]piperazine-1-carboxylate;
tert-butyl 4-[5-[(3-carbamoyl-4-pyridyl)methoxy]pyrimidin-2-yl]piperazine-1-carboxylate;
(3-methyloxetan-3-yl) (3R)-4-[5-[[4-(2-hydroxyethylcarbamoyl)-3-methyl-phenyl]methoxy]pyrimidin-2-yl]-3-methyl-piperazine-1-carboxylate; and
(3-methyloxetan-3-yl) (3R)-4-[[2-cyano-4-[2-hydroxypropyl(methyl)carbamoyl]-phenyl]methoxy]pyrimidin-2-yl]-3-methyl-piperazine-1-carboxylate;

In one embodiment of the present invention these compounds are excluded from the scope of the claims by means of a proviso.

The invention will now be illustrated by the following non-limiting examples in which, unless stated otherwise:

### EXAMPLES

The invention will now be illustrated by the following Examples in which, unless stated otherwise:
(i) temperatures are given in degrees Celsius (° C.); operations were carried out at room or ambient temperature, that is, at a temperature in the range of 18-25° C. and under an atmosphere of an inert gas such as argon;
(ii) evaporation of solvent was carried out using a rotary evaporator under reduced pressure (600-4000 Pa; 4.5-30 mmHg) with a bath temperature of up to 60° C.;
(iii) chromatography means flash chromatography on silica gel;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data (¹H) is in the form of delta values for major diagnostic protons, given in parts per million (ppm) relative to tetramethylsilane (TMS), determined at 300 or 400 MHz (unless otherwise stated) using perdeuterio dimethyl sulfoxide (DMSO-d₆) as solvent, unless otherwise stated; peak multiplicities are shown thus: s, singlet; d, doublet; dd, doublet of doublets; dt, doublet of triplets; dm, doublet of multiplets; t, triplet, m, multiplet; br, broad;
(vii) chemical symbols have their usual meanings; SI units and symbols are used;
(viii) solvent ratios are given in volume:volume (v/v) terms;
(ix) mass spectra (MS) were run with an electron energy of 70 electron volts in the chemical ionisation (CI) mode using a direct exposure probe; where indicated ionisation was effected by electron impact (EI), fast atom bombardment (FAB) or electrospray (ESP); values for m/z are given; generally, only ions which indicate the parent mass are reported;
(x) where a synthesis is described as being analogous to that described in a previous example the amounts used are the millimolar ratio equivalents to those used in the previous example;
(xi) The following methods were used throughout the Examples and intermediates for liquid chromatography (LC)/mass spectral (MS) analysis:—
HPLC: Agilent 1100 or Waters Alliance HT (2790 & 2795)
Mass Spectrometer: Waters ZQ ESCi.
(xii) The following abbreviations may be used below or in the process section hereinbefore:
   Et₂O diethyl ether
   DMF dimethylformamide
   DCM dichloromethane
   DME 1,2-dimethoxyethane
   MeOH methanol
   EtOH ethanol
   H₂O water
   TFA trifluoroacetic acid
   THF tetrahydrofuran
   DMSO dimethylsulfoxide
   HOBt 1-hydroxybenzotriazole
   EDCI (EDAC) 1-ethyl-3-(3-dimethylaminopropyl)carbodi-imide hydrochloride
   DIPEA diisopropylethylamine
   DEAD diethyl azodicarboxylate
   EtOAc ethyl acetate
   NaHCO₃ sodium bicarbonate
   K₃PO₄ potassium phosphate
   MgSO₄ magnesium sulfate
   PS polymer supported
   BINAP 2,2′-bis(diphenylphosphino)-1,1′binaphthyl
   Dppf 1,1′-bis(diphenylphosphino)ferrocene
   dba dibenzylidineacetone
   PS-CDI polymer supported carbonyldiimidazole
   INT Intermediate
   rel vols relative volumes (mL per gram)
   rt or RT ambient temperature
   ppt precipitate
   aq. aqueous
   ex Example

### Example 1

### Tert-butyl 4-(5-(4-(methylsulfonyl)benzyloxy)pyrimidin-2-yl)piperazine-1-carboxylate

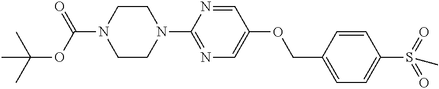

Cesium carbonate (2.092 g, 6.42 mmol) was added to tert-butyl 4-(5-hydroxypyrimidin-2-yl)piperazine-1-carboxylate (Intermediate 1)(0.6 g, 2.14 mmol) and 1-(bromomethyl)-4-(methylsulfonyl)benzene (0.587 g, 2.35 mmol) in DMF (10 mL). The resulting mixture was stirred at 40° C. for 2 hours. The reaction mixture was quenched with water (150 mL), extracted with Et₂O (2×200mL), the organic layer was dried over MgSO₄, filtered and evaporated to afford a cream solid. Upon addition of water and EtOAc/ether, a white solid was filtered off and dried. The cream solid was triturated with DCM to give a white solid. The two white solids were combined to give tert-butyl 4-(5-(4-(methylsulfonyl)-benzyloxy)pyrimidin-2-yl)piperazine-1-carboxylate (0.496 g, 52%).

¹H NMR (400.132 MHz, DMSO) 1.42 (9H, s), 3.22 (3H, s), 3.37-3.42 (4H, m), 3.59-3.65 (4H, m), 5.25 (2H, s), 7.71 (2H, d), 7.96 (2H, d), 8.30 (2H, s).

m/z (ES+) (M−H)−=447; HPLC tR=3.12 min

The following Examples were prepared in a similar manner to Example 1, using Intermediate 1 and an appropriate bromide or chloride starting material:

| Structure | Ex | Name | 1H NMR δ | MS |
|---|---|---|---|---|
| 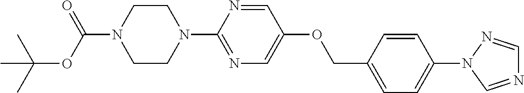 | 2 | tert-butyl 4-(5- (4-(1H-1,2,4- triazol-1- yl)benzyl- oxy)pyrimidin- 2-yl)piperazine- 1-carboxylate | 1H NMR (400.132 MHz, DMSO) 1.40 (9 H, s), 3.37 (4 H, t), 3.60 (4 H, t), 5.17 (2 H, s), 7.61 (2 H, d), 7.87 (2 H, d), 8.23 (1 H, s), 8.28 (2 H, s), 9.29 (1 H, s) | m/z (ES+) M - H)- = 436; HPLC tR = 3.19 min. |
| 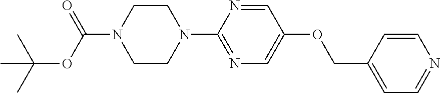 | 3 | tert-butyl 4-(5- (pyridin-4- ylmethoxy) pyrimidin-2- yl)piperazine-1- carboxylate | 1H NMR (400.132 MHz, CDCl3) 1.48 (9 H, s), 3.47- 3.50 (4 H, m), 3.69-3.73 (4 H, m), 5.04 (2 H, s), 7.32 (2 H, d), 8.12 (2 H, s), 8.63 (2 H, d) | m/z (ES+) M + H)+ = 372; HPLC tR= 2.31 min. |
| 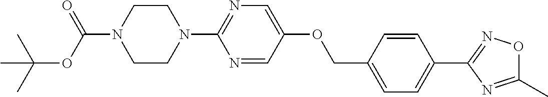 | 4 | tert-butyl 4-(5- (4-(5-methyl- 1,2,4-oxadiazol-3- yl)benzyloxy) pyrimidin-2- yl)piperazine-1- carboxylate | | |
| 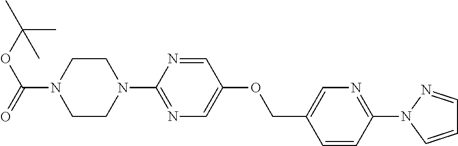 | 5 | tert-butyl 4-(5- ((6-(1H-pyrazol- 1-yl)pyridin-3- yl)methoxy) pyrimidin-2- yl)piperazine-1- carboxylate | 1H NMR (500 MHz, CDCl3) 8.57 (s, 1 H), 8.43 (s, 1 H), 8.16 (s, 2 H), 8.03 (d, J = 8.4, 1 H), 7.89 (s, 1 H), 7.75 (s, 1 H), 6.48 (s, 1 H), 5.05 (s, 2 H), 3.75 (s, 4 H), 3.50 (s, 4 H), 1.49 (s, 9 H) | |
| 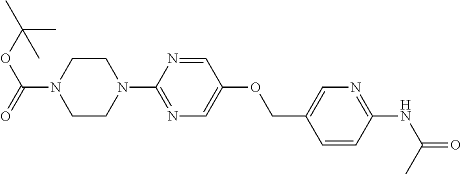 | 6 | tert-butyl 4-(5- ((6-acetamido- pyridin-3- yl)methoxy) pyrimidin-2- yl)piperazine-1- carboxylate | 1H NMR (500 MHz, CDC13) 8.45 (m, 2 H), 8.26 (m, 1 H), 8.13 (s, 2 H), 7.95 (m, 1 H), 5.01 (s, 2 H), 3.74 (s, 4 H), 3.50 (s, 4 H), 2.30 (s, 3 H), 1.49 (s, 9 H) | |

### Example 7

### Tert-butyl 4-(5-(4-(1H-tetrazol-1-yl)benzyloxy)pyrimidin-2-yl)piperazine-1-carboxylate

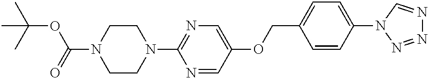

Diisopropyl azodicarboxylate (0.263 mL, 1.34 mmol) was added to a stirred solution of tert-butyl 4-(5-hydroxypyrimidin-2-yl)piperazine-1-carboxylate (Intermediate 1) (0.3 g, 1.07 mmol), and triphenylphosphine (0.421 g, 1.61 mmol) in THF (20 mL) under nitrogen. The resulting solution was stirred at 20° C. for 30 minutes and then (4-(1H-tetrazol-1-yl)phenyl)methanol (0.236 g, 1.34 mmol) was added. The resulting solution was stirred at rt overnight under nitrogen. The solvent was evaporated and the residue diluted with EtOAc and brine. A white ppt was filtered off and dried under vacuum. The aqueous layer was extracted with EtOAc (50 mL) and the combined organics were concentrated in vacuo to afford crude product. The crude product was purified by flash silica chromatography, elution gradient 20 to 80% EtOAc in isohexane. Pure fractions were evaporated to dryness and triturated with DCM/isohexane and combined with the ppt from above to afford tert-butyl 4-(5-(4-(1H-tetrazol-1-yl)benzyloxy)pyrimidin-2-yl)piperazine-1-carboxylate (0.201 g, 43%) as a white solid. ¹H NMR (400.132 MHz, DMSO) 1.41 (9H, s), 3.35-3.39 (4H, m), 3.58-3.63 (4H, m), 5.22 (2H, s), 7.70 (2H, d), 7.93 (2H, d), 8.29 (2H, s), 10.09 (1H s) m/z (ES+) (M+Na)+=461; HPLC tR=3.13 min.

The following Examples were prepared in a similar manner to Example 7, using

Intermediate 1 and the appropriate alcohol starting material:

| Structure | Ex | Name | 1H NMR δ | MS |
|---|---|---|---|---|
| 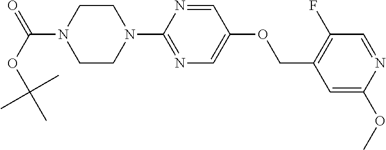 | 8 | tert-butyl 4-(5- ((5-fluoro-2- methoxy- pyridin-4- yl)methoxy) pyrimidin-2- yl)piperazine- 1-carboxylate | 1H NMR (400.13 MHz, CDCl3) 1.48 (9 H, s), 3.47- 3.50 (4 H, m), 3.70-3.73 (4 H, m), 3.91 (3 H, s), 5.06 (2 H, s), 6.87 (1 H, d), 7.99 (1 H, d), 8.13 (2 H, s) | m/z (ES-) (M - H)- = 418; HPLC tR = 3.42 min. |
| 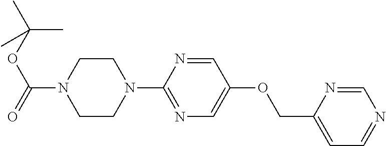 | 9 | tert-butyl 4-(5- (pyrimidin-4- ylmethoxy) pyrimidin-2- yl)piperazine- 1-carboxylate | 1H NMR (400.13 MHz, DMSO-d6) 1.41 (9 H, s), 3.35-3.40 (4 H, m), 3.60- 3.63 (4 H, m), 5.22 (2 H, s), 7.63-7.65 (1 H, m), 8.32 (2 H,s), 8.83 (1 H, d), 9.16 (1 H, d) | m/z (ES+) (M + H)+ = 373.16; HPLC tR = 2.88 min. |
| 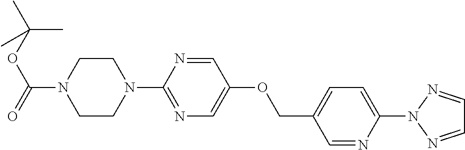 | 10 | tert-butyl 4-(5- ((6-(1H-1,2,4- triazol-1- yl)pyridin-3- yl)methoxy) pyrimidin-2- yl)piperazine- 1-carboxylate | 1H NMR (400.132 MHz, CDCl3) 1.48 (9 H, s), 3.49 (4 H, t), 3.72 (4 H, t), 5.08 (2 H, s), 7.95 (2 H, d), 8.10 (1 H, s), 8.14 (2 H, s), 8.49 (1 H, s), 9.18 (1 H, s) | m/z (ES+) (M - Boc) = 339.36; HPLC tR= 3.19 min. |
| 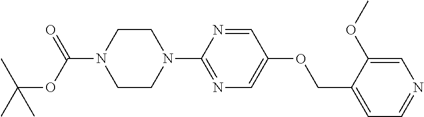 | 11 | tert-butyl 4-(5- ((3-methoxy- pyridin-4- yl)methoxy) pyrimidin-2- yl)piperazine- 1-carboxylate | 1H NMR (400 MHz, DMSO) 1.4 (s, 9 H), 3.4 (t, 4 H), 3.6 (t, 4 H), 3.95 (s, 3 H), 5.1 (s, 2 H), 7.4 (d, 1 H), 8.2 (d, 1 H), 8.25 (s, 2 H), 8.4 (s, 1 H). | m/z (ES+) M + H)+ = 402.20; HPLC tR = 3.30 min. |
| 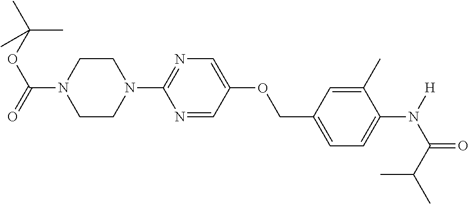 | 12 | tert-butyl 4-(5- (4-isobutyr- amido-3- methyl- benzyloxy) pyrimidin-2- yl)piperazine- 1-carboxylate | 1H NMR (CDCl3) 1.2 (d, 6 H), 1.4 (s, 9 H), 2.2 (s, 3 H), 2.5 (dq, 1 H), 3.4 (t, 4 H), 3.6 (t, 4 H), 4.9 (s, 2 H), 6.9 (br, 1 H), 7.15 (d, 2 H), 7.8 (d, 1 H) and 8.0 (s, 2 H). | m/z (ES+) (M + H)+ = 470; HPLC tR = 3.36 min |
| 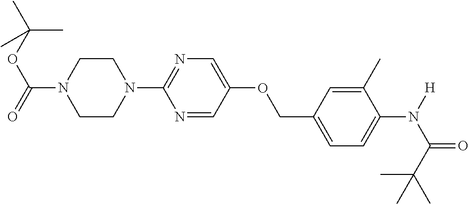 | 13 | tert-butyl 4-(5- (3-methyl-4- pivalamido- benzyloxy) pyrimidin-2- yl)piperazine- 1-carboxylate | 1H NMR (CDCl3) 1.25 (s, 9 H), 1.4 (s, 9 H), 2.2 (s, 3 H), 3.4 (t, 4 H), 3.6 (t, 4 H), 4.9 (s, 2 H), 7.1 (s, 1 H), 7.15 (d, 1 H), 7.85 (d, 1 H) and 8.05 (s, 2 H). | m/z (ES+) (M + H)+ = 484; HPLC tR = 3.47 min |
| 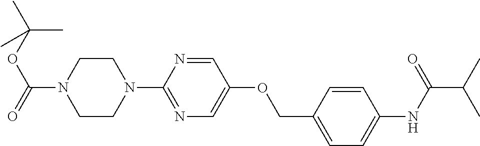 | 14 | tert-butyl 4-(5- (4-isobutyramido- benzyloxy) pyrimidin-2- yl)piperazine- 1-carboxylate | 1H NMR (400.13 MHz, CDCl3) 1.24-1.27 (6 H, m), 1.48 (9 H, s), 2.51 (1 H, septet), 3.47-3.49 (4 H, m), 3.68-3.71 (4 H, m), 4.97 (2 H, s), 7.16 (1 H, s), 7.34 (2 H, d), 7.55 (2 H, d), 8.09 (2 H, s) | m/z (ES+) (M + H)+ = 456.36; HPLC tR = 3.37 min |
| 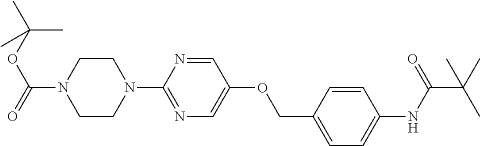 | 15 | tert-butyl 4-(5- (4-pivalamido- benzyloxy) pyrimidin-2- yl)piperazine- 1-carboxylate | 1H NMR (400.13 MHz, CDCl3) 1.32 (9 H, s), 1.48 (9 H, s), 3.46-3.49 (4 H, m), 3.68-3.71 (4 H, m), 4.98 (2 H, s), 7.32-7.35 (3 H, m), 7.54-7.56 (2 H, m), 8.09 (2 H, s) | m/z (ES+) (M + H)+ = 470.45; HPLC tR = 3.47 min |
| 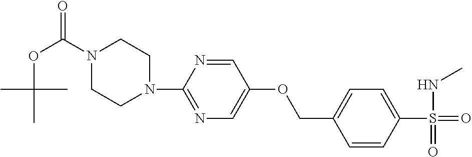 | 16 | tert-butyl 4-(5- (4-(N-methyl- sulfamoyl) benzyloxy) pyrimidin-2- yl)piperazine- 1-carboxylate | 1H NMR (400.13 MHz, CDCl3) 1.48 (9 H, s), 2.68 (3 H, d), 3.47-3.50 (4 H, m), 3.69-3.73 (4 H, m), 4.37 (1 H, dd), 5.09 (2 H, s), 7.57 (2 H, d), 7.89 (2 H, d), 8.12 (2 H, s) | m/z (ESI-) (M - H)- = 462; HPLC tR = 3.17 min |
| 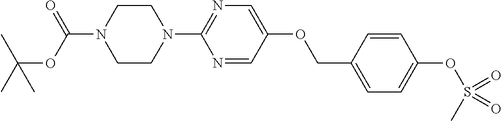 | 17 | tert-butyl 4-(5- (4-(methyl- sulfonyloxy) benzyloxy) pyrimidin-2- yl)piperazine- 1-carboxylate | 1H NMR (400 MHz, DMSO) 1.4 (s, 9 H), 3.35 (m, 7 H), 3.6 (t, 4 H), 5.1 (s, 2 H), 7.35 (d, 2 H), 7.55 (d, 2 H), 8.3 (s, 2 H). | m/z (ES+) (M + H)+ = 465.11; HPLC tR = 3.26 min. |

### Example 18

### (R)-Tert-butyl 3-methyl-4-(5-(4-(methylsulfonyl)benzyloxy)pyrimidin-2-yl)piperazine-1-carboxylate

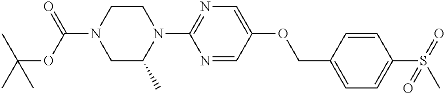

To a stirred solution of (R)-tert-butyl 4-(5-hydroxypyrimidin-2-yl)-3-methylpiperazine-1-carboxylate (Intermediate 10) (5.0 g, 16.99 mmol) and 1-(chloromethyl)-4-(methylsulfonyl)benzene (3.65 g, 17.84 mmol) in acetonitrile (170 mL) at ambient temperature was added potassium carbonate (7.04 g, 50.96 mmol). The mixture was heated under reflux at 80° for 2 hours, cooled to ambient temperature, the acetonitrile was evaporated in vacuo to give a residue which was partitioned between ethyl acetate (160 mL) and water (80 mL), the ethyl acetate layer was washed with brine, dried (MgSO₄) and evaporated in vacuo to a residue which was crystallised from ethyl acetate/isohexane to give (R)-tert-butyl 3-methyl-4-(5-(4-(methylsulfonyl)benzyloxy)pyrimidin-2-yl)piperazine-1-carboxylate (7.25 g, 92%). ¹H NMR (DMSO d6 @ 100°) 1.1 (d, 3H), 1.45 (s, 9H), 2.9 (m, 1H), 3.1 (m, 2H), 3.2 (s, 3H), 3.8 9d, 1H), 3.9 (d, 1H), 4.25 (d, 1H), 4.7 (br, 1H), 5.2 (s, 2H), 7.7 (d, 2H), 7.9 (d, 2H) and 8.25 (s, 2H).m/z (ES−) (M−H)−=461; HPLC tR=2.63 min

### Example 19

### (R)-Tert-butyl 3-methyl-4-(5-(pyridin-4-ylmethoxy)pyrimidin-2-yl)piperazine-1-carboxylate

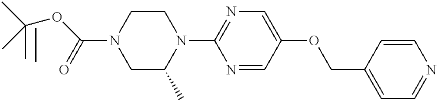

To a mixture of (R)-tert-butyl 4-(5-hydroxypyrimidin-2-yl)-3-methylpiperazine-1-carboxylate (Intermediate 10) (0.2 g, 0.68 mmol), 4-(chloromethyl)pyridine hydrochloride (0.123 g, 0.75 mmol) and cesium carbonate (0.664 g, 2.04 mmol) under an atmosphere of nitrogen was added DMF (6 mL). The mixture was stirred at ambient temperature for 3 days. The reaction mixture was diluted with EtOAc (25 mL), and washed sequentially with water (20 mL) and saturated brine (25 mL). The organic layer was dried over Na₂SO₄, filtered and evaporated to afford crude product. The crude product was purified by flash silica chromatography, elution gradient 1 to 5% MeOH in DCM. Pure fractions were evaporated to dryness to afford (R)-tert-butyl 3-methyl-4-(5-(pyridin-4-ylmethoxy)pyrimidin-2-yl)piperazine-1-carboxylate (0.134 g, 51%) as a white solid.

¹H NMR (400.132 MHz, CDCl₃) 1.15 (3H, d), 1.48 (9H, s), 2.84-2.95 (1H, m), 3.05-3.19 (2H, m), 3.81-4.19 (2H, m), 4.28-4.35 (1H, m), 4.70-4.80 (1H, m), 5.04 (2H, s), 7.33 (2H, d), 8.13 (2H, s), 8.62-8.64 (2H, m). m/z (ES+) (M+H)+=386; HPLC tR=2.49 min

### Example 20

### (R)-Tert-butyl 4-(5-(4-(1H-tetrazol-1-yl)benzyloxy)pyrimidin-2-yl)-3-methylpiperazine-1-carboxylate

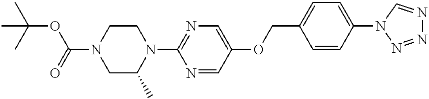

Diisopropyl azodicarboxylate (0.176 mL, 0.89 mmol) was added to a stirred solution of (R)-tert-butyl 4-(5-hydroxypyrimidin-2-yl)-3-methylpiperazine-1-carboxylate (Intermediate 10)(0.21 g, 0.71 mmol), and triphenylphosphine (0.281 g, 1.07 mmol) in THF (15 mL) under nitrogen. The resulting solution was stiffed at 20° C. for 30 minutes and then (4-(1H-tetrazol-1-yl)phenyl)methanol (0.157 g, 0.89 mmol) was added. The resulting solution was stirred at rt overnight under nitrogen. The solvent was evaporated and the residue diluted with EtOAc and brine. A white ppt was filtered off and dried under vacuum. The aqueous layer was extracted with EtOAc (50 mL) and the combined organics were concentrated in vacuo to afford crude product. The crude product was purified by flash silica chromatography, elution gradient 1 to 4% MeOH in DCM. The crude product was re-purified by flash silica chromatography, elution gradient 40 to 100% EtOAc in isohexane. Pure fractions were evaporated to dryness to afford (R)-tert-butyl 4-(5-(4-(1H-tetrazol-1-yl)benzyloxy)pyrimidin-2-yl)-3-methylpiperazine-1-carboxylate (0.087 g, 27%) as a white solid. ¹H NMR (400.132 MHz, CDCl₃) 1.15 (3H, d), 1.49 (9H, s), 2.85-3.00 (1H, m), 3.07-3.19 (2H, m), 3.83-4.20 (2H, m), 4.32 (1H, d), 4.71-4.82 (1H, m), 5.11 (2H, s), 7.64 (2H, d), 7.74 (2H, d), 8.14 (2H, s), 8.99 (1H, s).m/z (ES+) (M+H)+=453; HPLC tR=3.32 min

The following Example was prepared in a similar manner to Example 19, using Intermediate 10 and Intermediate 63:

| Structure | Ex | Name | 1H NMR δ | MS |
|---|---|---|---|---|
| 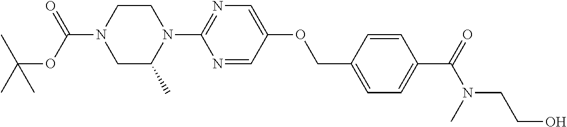 | 21 | (R)-tert-butyl 4-(5-(4-((2- hydroxyethyl) (methyl)carba- moyl)benzyl- oxy) pyrimidin-2- yl)-3-methyl- piperazine- 1- carboxylate | 1H NMR (CDCl3) 1.1 (d, 3H), 1.4 (s, 9H), 2.8-3.1 (m, 7H), 3.65 (br, 2H), 3.85 (br, 2H), 3.9-4.1 (br, 2H), 4.25 (d, 1H), 4.7 (br, 1H), 5.0 (s, 2H), 7.4 m, 4H) and 8.1 (s, 2H). | m/z (ES+) (M − Boc)+ = 386; HPLC tR = 2.20 min |

### Example 22

### (S)-Tert-butyl 2-methyl-4-(5-(4-(methylsulfonyl)benzyloxy)pyrimidin-2-yl)piperazine-1-carboxylate

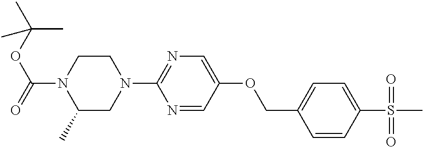

Cesium carbonate (2192 mg, 6.73 mmol) was added to (S)-tert-butyl 4-(5-hydroxypyrimidin-2-yl)-2-methylpiperazine-1-carboxylate (Intermediate 2)(660 mg, 2.24 mmol) and 1-(chloromethyl)-4-(methylsulfonyl)benzene (459 mg, 2.24 mmol) in acetonitrile (15 mL). The resulting mixture was stirred at room temperature for 18 hours. The reaction mixture was quenched with water (50 mL), extracted with EtOAc (2×100mL), the organic layer was dried over MgSO₄, filtered and evaporated to afford a yellow oil (700 mg). The crude product was purified by flash silica chromatography, elution gradient 20 to 100% EtOAc in isohexane. Pure fractions were evaporated to dryness to afford (S)-tert-butyl 2-methyl-4-(5-(4-(methylsulfonyl)benzyloxy)pyrimidin-2-yl)piperazine-1-carboxylate (29%) as a colourless solid. ¹H NMR (400.132 MHz, CDCl₃) 1.15 (3H, d), 1.48 (9H, s), 2.92-3.01 (1H, m), 3.06 (3H, s), 3.11-3.20 (2H, m), 3.86-3.94 (1H, m), 4.27-4.38 (2H, m), 4.41-4.48 (1H, m), 5.11 (2H, s), 7.62 (2H, d), 7.97 (2H, d), 8.11 (2H, s). m/z (ES+) (M−tBu)+=407.32; HPLC tR=3.18 min

The following Examples were prepared in a similar manner to Example 22, using the Intermediates stated and 1-(chloromethyl)-4-(methylsulfonyl)benzene:

| Structure and INT | Ex | Name | 1H NMR δ | MS |
|---|---|---|---|---|
| 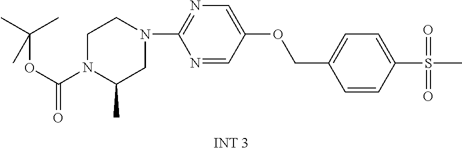 | 23 | (R)-tert-butyl 2-methyl-4-(5- (4- (methylsulfonyl) benzyloxy) pyrimidin-2- yl)piperazine- 1-carboxylate | 1H NMR (400.13 MHz, CDCl3) 1.15 (3H, d), 1.48 (9H, s), 2.96-3.01 (1H, m), 3.06 (3H, s), 3.13- 3.18 (2H, m), 3.90 (1H, dt), 4.33-4.37 (2H, m), 4.41-4.47 (1H, m), 5.11 (2H, s), 7.62 (2H, d), 7.97 (2H, d), 8.11 (2H, s) | m/z (ESI−) (M − H)− = 461; HPLC tR = 3.20 min. |
| 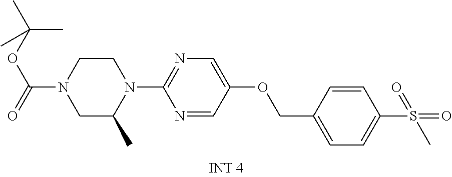 | 24 | (S)-tert-butyl 3-methyl-4-(5- (4- (methylsulfonyl) benzyloxy) pyrimidin-2- yl)piperazine- 1-carboxylate | 1H NMR (CDCl3) 1.1 (d, 3H), 1.4 (s, 9H), 2.85 (br, 1H), 3.0 (s, 3H), 3.0-3.1 (m, 2H), 3.85 (br, 2H), 4.25 (d, 1H), 4.7 (s, 1H), 5.05 (s, 2H), 7.55 (d, 2H), 7.9 (d, 2H) and 8.05 (s, 2H). | m/z (ES+) (M − tBu)+ = 407; HPLC tR = 3.20 min |
| 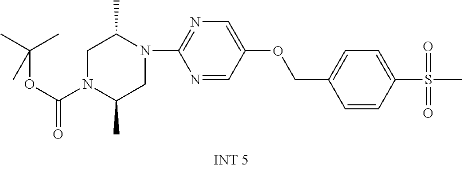 | 25 | (2R,5S)-tert- butyl 2,5- dimethyl-4-(5- (4- (methylsulfonyl) benzyloxy) pyrimidin-2- yl)piperazine- 1-carboxylate | 1H NMR (400.132 MHz, CDCl3) 1.10-1.20 (6H, m), 1.48 (9H, s), 3.06 (3H, s), 3.21-3.36 (2H, m), 3.69-3.84 (1H, m), 4.16- 4.50 (2H, m), 4.73-4.82 (1H, m), 5.11 (2H, s), 7.62 (2H, d), 7.98 (2H, d), 8.11 (2H, s) | m/z (ES+) (M + H)+ = 477; HPLC tR = 3.19 min. |
| 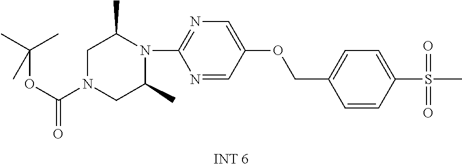 | 26 | (3R,5S)-tert- butyl 3,5- dimethyl-4-(5- (4- (methylsulfonyl) benzyloxy) pyrimidin-2- yl)piperazine- 1-carboxylate | 1H NMR (400.13 MHz, CDCl3) 1.22 (6H, d), 1.50 (9H, s), 3.04 (2H,bs), 3.06 (3H, s), 4.00 (2H,bs), 4.66 (2H, bs), 5.11 (2H, s), 7.62 (2H, d), 7.98 (2H, d), 8.14 (2H, s) | m/z (ESI−) (M − H)− = 475; HPLC tR = 3.29 min. |
| 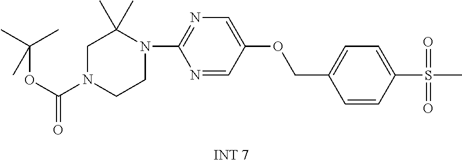 | 27 | tert-butyl 3,3- dimethyl-4-(5- (4- (methylsulfonyl) benzyloxy) pyrimidin-2- yl)piperazine- 1-carboxylate | 1H NMR (400.13 MHz, CDCl3) 1.48 (9H, s), 3.06 (3H, s), 3.46-3.55 (4H, m), 3.98 (2H, t), 5.12 (2H, s), 7.62 (2H, d), 7.96 (2H, d), 8.11 (2H, s) | m/z (ESI+) (M + H)+ = 477; HPLC tR = 3.88 min. |
| 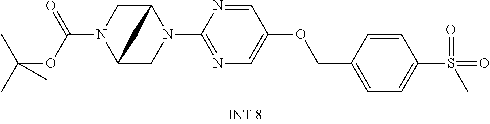 | 28 | (1R,4R)-tert- butyl 5-(5-(4- (methylsulfonyl) benzyloxy) pyrimidin-2-yl)- 2,5- diazabicyclo [2.2.1]heptane-2- carboxylate | 1H NMR (400 MHz, DMSO) 1.35 (m, 9H), 1.9 (m, 2H), 3.1 (d, 1H), 3.2 (s, 3H), 3.35 (m, 2H), 3.45 (m, 1H), 4.4 (m, 1H), 4.75 (m, 1H), 5.2 (s, 2H), 7.7 (d, 2H), 7.95 (d, 2H), 8.25 (s, 2H). | RT = 2.86, MH− = 459.13 |
| 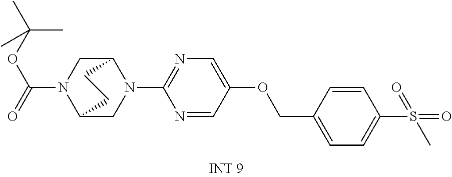 | 29 | (1S,4S)-tert- butyl 5-(5-(4- (methylsulfonyl) benzyloxy) pyrimidin-2-yl)- 2,5- diazabicyclo [2.2.2]octane-2- carboxylate | 1H NMR (CDCl3) 1.4 (s, 9H), 1.75 (m, 2H), 1.95 (m, 2H), 3.0 (s, 3H), 3.45 (m, 1H), 3.5 (m, 2H), 3.7 (m, 1H), 4.2-4.35 (d, 1H), 4.8 (d, 1H), 5.0 (s, 2H), 7.55 (d, 2H), 7.9 (d, 2H) and 8.0 (s, 2H). | m/z (ES+) (M − tBu)+ = 419; HPLC tR = 3.20 min |

### Example 30

### Tert-butyl 4-(5((6-(methylsulfonyl)pyridin-3-yl)methoxy)pyrimidin-2-yl)piperazine-1-carboxylate

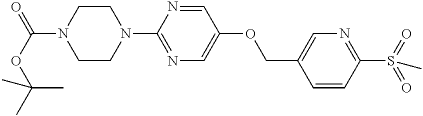

Sodium methanesulfinate (497 mg, 4.87 mmol) was added to tert-butyl 4-(5-((6-bromopyridin-3-yl)methoxy)pyrimidin-2-yl)piperazine-1-carboxylate (Intermediate 20) (548 mg, 1.22 mmol), copper (1) trifluoromethanesulfonate toluene complex (78 mg, 0.12 mmol) and N,N′-dimethylethylenediamine (0.027 mL, 0.24 mmol) in DMSO (20 mL) at 20° C. under nitrogen. The resulting solution was stirred at 130° C. for 90 minutes. The reaction mixture was diluted with EtOAc (100 mL), and washed sequentially with water (100 mL) and saturated brine (75 mL). The organic layer was dried over MgSO₄, filtered and evaporated to afford crude product. The crude product was purified by flash silica chromatography, elution gradient 50 to 100% EtOAc in isohexane. Pure fractions were evaporated to dryness to afford tert-butyl 4-(5-((6-(methylsulfonyl)pyridin-3-yl)methoxy)pyrimidin-2-yl)piperazine-1-carboxylate (385 mg, 70%) as a yellow solid.

¹H NMR (400.132 MHz, CDCl₃) 1.48 (9H, s), 3.25 (3H, s), 3.49 (4H, t), 3.73 (4H, t), 5.14 (2H, s), 8.00-8.06 (1H, m), 8.10-8.15 (3H, m), 8.74-8.78 (1H, m). m/z (ES+)(M+H)+=450.38; HPLC tR=2.96 min

The following Examples were prepared in a similar manner to Example 30, using the Intermediate listed and sodium methanesulfinate:

| Structure and INT | Ex | Name | 1H NMR δ | MS |
|---|---|---|---|---|
| 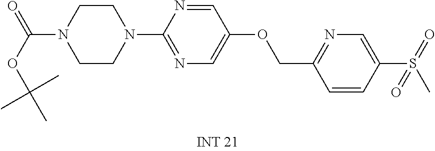 | 31 | tert-butyl 4-(5- ((5- (methylsulfonyl) pyridin-2- yl)methoxy) pyrimidin-2- yl)piperazine- 1-carboxylate | 1H NMR (400.13 MHz, CDCl3) 1.48 (9H, s), 3.12 (3H, s), 3.47-3.50 (4H, m), 3.71-3.73 (4H, m), 5.24 (2H, s), 7.74-7.77 (1H, m), 8.16 (2H, s), 8.26- 8.29 (1H, m), 9.12-9.13 (1H, m | m/z (ES−) (M − H)− = 448; HPLC tR = 2.91 min. |
| 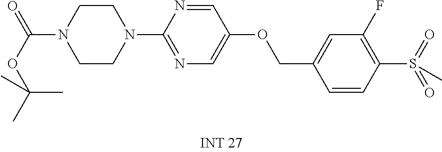 | 32 | tert-butyl 4-(5- (3-fluoro-4- (methylsulfonyl) benzyloxy) pyrimidin-2- yl)piperazine- 1-carboxylate | 1H NMR (400.13 MHz, CDCl3) 1.48 (9H, s), 3.23 (3H, d), 3.47-3.50 (4H, m), 3.71-3.73 (4H, m), 5.09 (2H, s), 7.33-7.37 (2H, m), 7.96-8.00 (1H, m), 8.12 (2H, s) | m/z (ES−) (M − H)− = 465.11; HPLC tR = 3.15 min. |
| 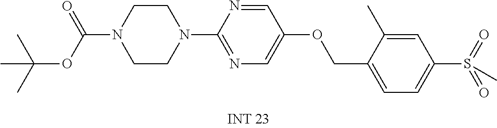 | 33 | tert-butyl 4-(5- (2-methyl-4- (methylsulfonyl) benzyloxy) pyrimidin-2- yl)piperazine- 1-carboxylate | 1H NMR (400.132 MHz, CDCl3) 1.48 (9H, s), 2.45 (3H, s), 3.05 (3H, s), 3.46- 3.51 (4H, m), 3.69-3.75 (4H, m), 5.05 (2H, s), 7.62 (1H, d), 7.78-7.82 (2H, m), 8.13 (2H, s) | m/z (ES+) (M − H)− = 461; HPLC tR = 3.19 min. |
| 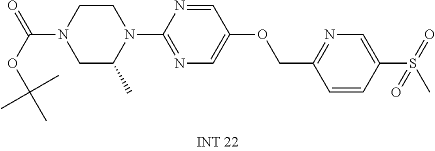 | 34 | (R)-tert-butyl 3-methyl-4-(5- ((5- (methylsulfonyl) pyridin-2- yl)methoxy) pyrimidin-2- yl)piperazine- 1-carboxylate | 1H NMR (400.132 MHz, CDCl3) 1.16 (3H, d), 1.49 (9H, s), 2.87-2.97 (1H, m), 3.08-3.20 (2H, m), 3.12 (3H, s), 3.82-4.19 (2H, m), 4.28-4.36 (1H, m), 4.72-4.81 (1H, m), 5.24 (2H, s), 7.76 (1H, d), 8.17 (2H, s), 8.27 (1H, dd), 9.12 (1H, d) | m/z (ES+) (M + H)+ = 464; HPLC tR = 3.07 min. |

### Example 35

### (R)-tert-butyl 4-(5-(2-fluoro-4-(methylsulfonyl)benzyloxy)pyrimidin-2-yl)-3-methylpiperazine-1-carboxylate

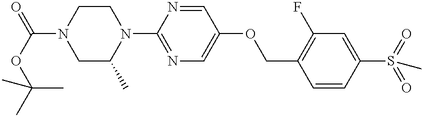

Copper (I) iodide (2.58 μl, 0.07 mmol) was added to (R)-tert-butyl 4-(5-(4-bromo-2-fluorobenzyloxy)pyrimidin-2-yl)-3-methylpiperazine-1-carboxylate (Intermediate 29)(330 mg, 0.69 mmol), methanesulphinic acid sodium salt (84 mg, 0.82 mmol), L-proline (15.79 mg, 0.14 mmol) and sodium hydroxide (4.06 μl, 0.14 mmol) in DMSO (3.0 mL) under nitrogen. The resulting solution was degassed with nitrogen for 20 minutes and stirred at 80° C. for 2 days. It was cooled to room temperature and partitioned between 50% brine (50 mL) and ethyl acetate (150 mL). The aqueous portion was back extracted with ethyl acetate (2×100mL) and all of the combined organics were washed with brine (50 mL), dried (sodium sulphate), concentrated in vacuo and adsorbed onto silica. The crude product was purified by flash silica chromatography, elution gradient 0 to 75% EtOAc in isohexane. Pure fractions were evaporated to dryness to afford (R)-tert-butyl 4-(5-(2-fluoro-4-(methylsulfonyl)benzyloxy)pyrimidin-2-yl)-3-methylpiperazine-1-carboxylate (190 mg, 58%) as a white solid. ¹H NMR (400 MHz, DMSO) 1.05 (d, 3H), 1.4 (s, 9H), 2.85 (m, 1H), 3.05 (m, 2H), 3.25 (s, 3H), 3.8 (m, 1H), 3.95 (m, 1H), 4.2 (m, 1H), 4.7 (m, 1H), 5.25 (s, 2H), 7.8 (m, 3H), 8.3 (s, 2H). m/z (ES−) (M−H)−=479.15; HPLC tR=3.31 min.

The following Examples were prepared in a similar manner to Example 35, using the intermediate shown and an appropriate bromide or chloride starting material:

| Structure and INT | Ex | Name | 1H NMR δ | MS |
|---|---|---|---|---|
| 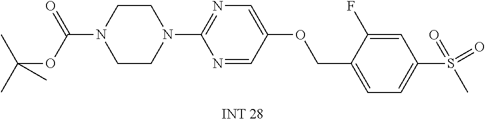 | 36 | tert-butyl 4- (5-(2- fluoro-4- (methylsulf- onyl)benzyl- oxy)pyrimi- din-2- yl)piperazine- 1- carboxylate | 1H NMR (DMSO d6) 1.4 (s, 9H), 3.3 (s, 3H), 3.4 (t, 4H), 3.6 (t, 4H), 5.25 (s, 2H), 7.8-7.9 (m, 3H) and 8.3 (s, 2H). | m/z (ES+) (M + H)+ = 467; HPLC tR = 2.51 min |
| 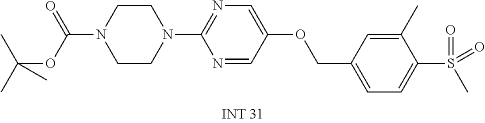 | 37 | tert-butyl 4- (5-(3- methyl-4- (methylsulf- onyl)benzyl- oxy)pyrimi- din-2- yl)piperazine- 1- carboxylate | 1H NMR (400 MHz, DMSO) 1.4 (s, 9H), 2.65 (s, 3H), 3.2 (s, 3H), 3.4 (m, 4H), 3.6 (m, 4H), 5.15 (s, 2H), 7.5 (m, 2H), 7.9 (d, 1H), 8.25 (s, 2H). | m/z (ES−) (M − H)− = 461.23; HPLC tR = 3.28 min |

### Example 38

### Tert-butyl 4-(5-((3-cyanopyridin-4-yl)methoxy)pyrimidin-2-yl)piperazine-1-carboxylate

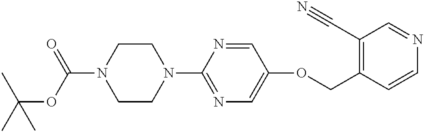

Tert-butyl 4-(5-((3-bromopyridin-4-yl)methoxy)pyrimidin-2-yl)piperazine-1-carboxylate (Intermediate 25) (0.95 g, 2.11 mmol), zinc cyanide (0.198 g, 1.69 mmol), tris(dibenzylideneacetone)dipalladium(0) (0.077 g, 0.08 mmol) and 4,5-bis(diphenylphosphino)-9,9-dimethylxanthene (0.098 g, 0.17 mmol) (xantphos) were suspended in DMF (20 mL) and sealed into a microwave tube (evacuated and purged with nitrogen). The reaction was heated to 130° C. for 60 minutes in the microwave reactor and cooled to RT. The reaction mixture was filtered through celite. The reaction mixture was diluted with EtOAc (100 mL), and washed sequentially with water (100 mL) and saturated brine (100 mL). The organic layer was dried over Na2SO4, filtered and evaporated to afford crude product. The crude product was purified by flash silica chromatography, is elution gradient 1 to 4% MeOH in DCM. This was recolumned by flash silica chromatography, elution gradient 20 to 80% EtOAc in isohexane. Pure fractions were evaporated to dryness to afford tert-butyl 4-(5-((3-cyanopyridin-4-yl)methoxy)pyrimidin-2-yl)piperazine-1-carboxylate (0.410 g, 49%) as a white solid. ¹H NMR (400.132 MHz, CDCl₃) 1.49 (9H, s), 3.46-3.53 (4H, m), 3.72-3.75 (4H, m), 5.21 (2H, s), 7.65 (1H, d), 8.17 (2H, s), 8.84 (1H, d), 8.90 (1H, s). m/z (ES+) M+=397; HPLC tR=3.04 min.

The following Example was prepared in a similar manner to Example 38, using the intermediate stated and zinc cyanide:

| Structure and INT | Ex | Name | 1H NMR δ | MS |
|---|---|---|---|---|
| 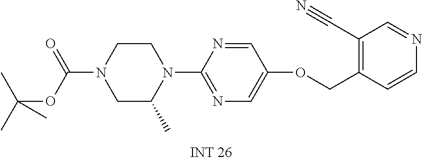 | 39 | (R)-tert- butyl 4-(5- ((3- cyanopyri- din-4- yl)methoxy) pyrimidin- 2-yl)-3- methylpiper- azine-1- carboxylate | 1H NMR (400.132 MHz, CDCl3) 1.16 (3H, d), 1.49 (9H, s), 2.86-2.97 (1H, m), 3.05- 3.22 (2H, m), 3.83-4.18 (2H, m), 4.30-4.39 (1H, m), 4.72- 4.84 (1H, m), 5.20 (2H, s), 7.66 (1H, d), 8.18 (2H, s), 8.84 (1H, d), 8.90 (1H, s) | m/z (ES+) (M + H)+ = 411; HPLC tR = 3.16 min. |

### Example 40

### 4-((2-(4-(5-Fluoropyrimidin-2-yl)piperazin-1-yl)pyrimidin-5-yloxy)methyl)nicotinonitrile

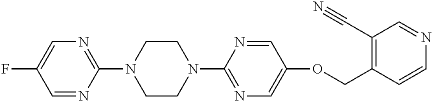

4-((2-(Piperazin-1-yl)pyrimidin-5-yloxy)methyl)nicotinonitrile hydrochloride (0.35 g, 1.05 mmol), 2-chloro-5-fluoropyrimidine (0.418 g, 3.16 mmol) and N-ethyl-N-isopropylpropan-2-amine (0.366 mL, 2.10 mmol) were dissolved in acetonitrile (15 mL) and sealed into a microwave tube. The reaction was heated to 100° C. for 3 hours in the microwave reactor and cooled to RT. Heated for a further 6 hours. The reaction was incomplete and further 2-chloro-5-fluoropyrimidine (0.418 g, 3.16 mmol) was added and the solution was stirred at 120° C. for a further 3 hours. The reaction was incomplete and further N-ethyl-N-isopropylpropan-2-amine (0.366 mL, 2.10 mmol)was added and the suspension was stiffed at 130° C. for a further 4 hours. The reaction mixture was diluted with EtOAc (50 mL), and washed sequentially with water (50 mL) and saturated brine (50 mL). The organic layer was dried over MgSO₄, filtered and evaporated to afford crude product. The crude product was purified by flash silica chromatography, elution gradient 1 to 5% MeOH in DCM. Pure fractions were evaporated to dryness and triturated with ether/DCM to afford 4-((2-(4-(5-fluoropyrimidin-2-yl)piperazin-1-yl)pyrimidin-5-yloxy)methyl)nicotinonitrile (0.118 g, 29%) as a white solid.

¹H NMR (400.132 MHz, CDCl₃) 3.85 (8H, s), 5.23 (2H, s), 7.66 (1H, d), 8.19 (2H, s), 8.23 (2H, s), 8.85 (1H, d), 8.91 (1H, s).m/z (ES+) (M+H)+=393; HPLC tR=2.34 min.

The following Examples were prepared in a similar manner to Example 40, using the Intermediates stated and an appropriate bromide or chloride starting material:

| Structure | Ex | Name | 1H NMR δ | MS |
|---|---|---|---|---|
| 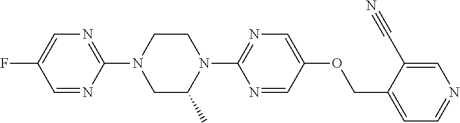 | 41 | (R)-4-((2-(4-(5- fluoropyrimidin- 2-yl)-2- methylpiperazin- 1-yl)pyrimidin- 5- yloxy)methyl) nicotinonitrile | 1H NMR (400.13 MHz, DMSO-d6) 1.05 (3H, d), 3.00- 3.09 (1H, m), 3.15- 3.28 (2H, m), 4.35 (1H, dt), 4.41-4.52 (2H, m), 4.75-4.82 (1H, m), 5.34 (2H, s), 7.74 (1H, dd), 8.34 (2H, s), 8.45 (2H, s), 8.87 (1H, d), 9.05 (1H, d) | m/z (ES+) (M + H)+ = 407.44; HPLC tR = 2.39 min. |
| 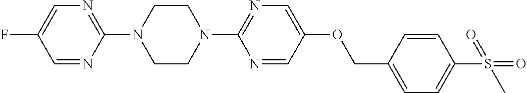 | 42 | 5-fluoro-2-(4-(5- (4- (methylsulfonyl) benzyloxy)pyrim- idin-2- yl)piperazin-1- yl)pyrimidine | 1H NMR (CDCl3). 3.0 (s, 3H), 3.8 (t, 8H), 5.05 (s, 2H), 7.5 (d, 2H), 7.9 (d, 2H), 8.05 (s, 2H) and 8.15 (s, 2H). | m/z (ES+) (M + H)+ = 445; HPLC tR = 3.07 min |

### Example 43

### Tert-butyl 4-(5-(4-((2-hydroxyethyl)(methyl)carbamoyl)benzyloxy)pyrimidin-2-yl)piperazine-1-carboxylate

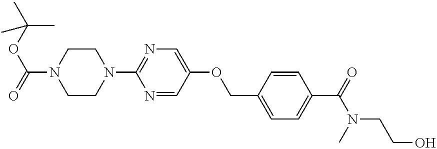

To a stirred suspension of 4-((2-(4-(tert-butoxycarbonyl)piperazin-1-yl)pyrimidin-5-yloxy)methyl)benzoic acid (Intermediate 37) (330 mg, 0.80 mmol) and 2-(methylamino)ethanol (59.8 mg, 0.80 mmol) in tetrahydrofuran (10.600 mL) under nitrogen at ambient temperature was added 4-(4,6-dimethoxy[1.3.5]triazin-2-yl)-4-methylmorpholinium chloride hydrate (242 mg, 0.88 mmol). The mixture was stirred at ambient temperature for 16 hours, the tetrahydrofuran was evaporated in vacuo to give a residue which was partitioned between 2M sodium carbonate solution (50 mL) and ethyl acetate (50 mL). The mixture was filtered through Celite and the aqueous layer extracted with ethyl acetate (2×50mL). The combined ethyl acetate extracts were washed with 2M sodium carbonate solution (2×50 mL), brine, dried (Na₂SO₄) and evaporated in vacuo to give a residue which was crystallised from ethyl acetate/isohexane to give tert-butyl 4-(5-(4-((2-hydroxyethyl)(methyl)carbamoyl)benzyloxy)pyrimidin-2-yl)piperazine-1-carboxylate (240 mg, 64%). ¹H NMR (DMSO-d6) 1.4 (s, 9H), 2.95 (s, 3H), 3.35 (t, 4H 3.4 (t, 2H), 3.6 (t, 2H), 3.65 (t, 4H), 4.4 (t, 1H), 5.15 (s, 2H), 7.4 (d, 2H), 7.45 (d, 2H) and 8.25 (s, 2H). m/z (ES+) (M-Boc)+=372; HPLC tR=3.04 min

The following Examples were prepared in a similar manner to Example 43, using the acid

Intermediate 37 and an appropriate amine starting material:

| Structure | Ex | Name | 1H NMR δ | MS |
|---|---|---|---|---|
| 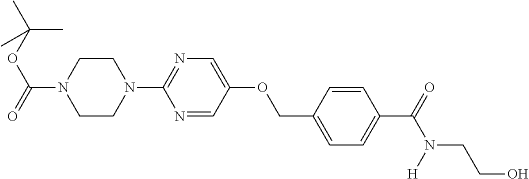 | 44 | tert-butyl 4-(5- (4-(2- hydroxyethyl- carbamoyl)benz- yloxy)pyrimi- din-2- yl)piperazine- 1-carboxylate | 1H NMR (DMSO d6) 1.4 (s, 9H), 3.3-3.4 (m, 6H), 3.45 (t, 2H), 3.65 (t, 4H), 4.35 (t, 1H), 5.15 (s, 2H), 7.5 (d, 2H), 7.85 (d, 2H), 8.05 (br, 1H) and 8.2 (s, 2H). | m/z (ES+) (M − Boc)+ = 358; HPLC tR = 2.98 min |
| 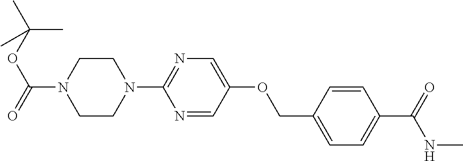 | 45 | tert-butyl 4-(5- (4- (methylcarba- moyl)benzyloxy) pyrimidin-2- yl)piperazine- 1-carboxylate | 1H NMR (400.13 MHz, DMSO-d6) 1.41 (9H, s), 2.77 (3H, d), 3.37 (4H, t), 3.58-3.61 (4H, m), 5.15 (2H, s), 7.49 (2H, d), 7.82- 7.84 (2H, m), 8.26 (2H, s), 8.40 (1H, d) | m/z (ES+) (M + H)+ = 428.24; HPLC tR = 3.09 min |
| 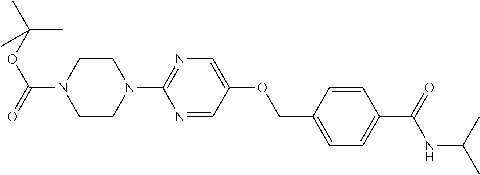 | 46 | tert-butyl 4-(5- (4- (isopropylcarba- moyl)benzyl- oxy)pyrimidin- 2- yl)piperazine- 1-carboxylate | 1H NMR (400.13 MHz, DMSO-d6) 1.15 (6H, d), 1.41 (9H, s), 3.35-3.38 (4H, m), 3.58-3.61 (4H, m), 4.05-4.09 (1H, m), 5.16 (2H, s), 7.48 (2H, d), 7.83-7.85 (2H, m), 8.17 (1H, d), 8.25 (2H, s) | m/z (ES−) (M − H)− = 454.24; HPLC tR = 3.29 min |
| 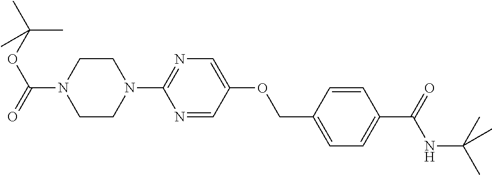 | 47 | tert-butyl 4-(5- (4-(tert- butylcarbamoyl) benzyloxy) pyrimidin-2- yl)piperazine- 1-carboxylate | 1H NMR (400.13 MHz, DMSO-d6) 1.36 (9H, s), 1.41 (9H, s), 3.37 (4H, t), 3.58-3.60 (4H, m), 5.16 (2H, s), 7.46 (2H, d), 7.71 (1H, s), 7.78-7.80 (2H, m), 8.25 (2H, s) | m/z (ES−) (M − H)− = 468.26; HPLC tR = 3.51 min |
| 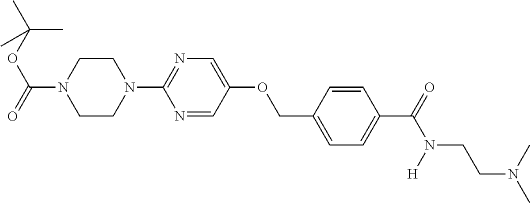 | 48 | tert-butyl 4-(5- (4-(2- (dimethylamino) ethylcarba- moyl)benzyloxy) pyrimidin-2- yl)piperazine- 1-carboxylate | 1H NMR (DMSO d6) 1.45 (s, 9H), 2.25 (s, 6H), 2.5 (t, 2H), 3.4 (t, 2H), 3.45 (t, 4H), 3.65 (t, 4H), 5.2 (s, 2H), 7.5 (d, 2H), 7.85 (d, 2H), 8.0 (br, 1H), and 8.25 (s, 2H). | m/z (ES−) (M − H)− = 483; HPLC tR = 3.16 min |
| 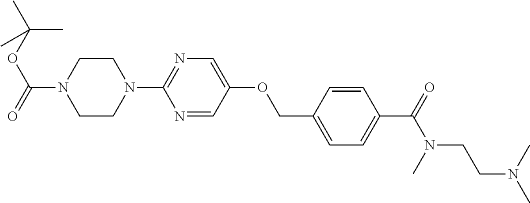 | 49 | tert-butyl 4-(5- (4-((2- (dimethylamino) ethyl)(methyl) carbamoyl) benzyloxy)pyrim- idin-2- yl)piperazine- 1-carboxylate | 1H NMR (DMSO d6) 1.4 (s, 6H), 2.1 (s, 6H), 2.45 (t, 2H), 2.95 (s, 3H), 3.4 (t, 6H), 3.65 (t, 4H), 5.15 (s, 2H), 7.35 (d, 2H), 7.5 (d, 2H) and 8.2 (s, 2H). | m/z (ES+) (M + H)+ = 499; HPLC tR = 3.30 min |
| 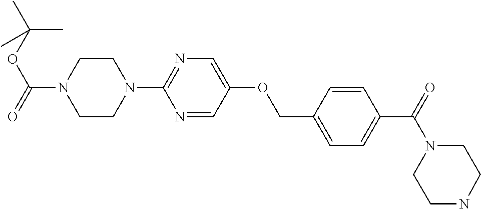 | 50 | tert-butyl 4-(5- (4-(piperazine- 1- carbonyl)benz- yloxy)pyrimi- din-2- yl)piperazine- 1-carboxylate | 1H NMR (DMSO d6) 1.4 (s, 9H), 2.65 (t, 4H), 3.3 (t, 8H), 3.6 (t, 4H), 5.05 (s, 2H), 7.3 (d, 2H), 7.4 (d, 2H), 8.1 (s, 1H) and 8.15 (s, 2H). | m/z (ES−) (M − H)− = 481; HPLC tR = 2.94 min |
| 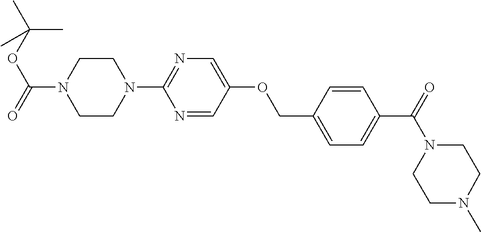 | 51 | tert-butyl 4-(5- (4-(4- methylpiper- azine-1- carbonyl)benz- yloxy)pyrimi- din-2- yl)piperazine- 1-carboxylate | 1H NMR (DMSO d6) 1.45 (s, 9H), 2.2 (s, 3H), 2.3 (t, 4H), 3.4 (t, 4H), 3.34 (t, 4H), 3.65 (t, 4H), 5.15 (s, 2H), 7.4 (d, 2H), 7.5 (d, 2H) and 8.25 (s, 2H). | m/z (ES+) (M + H)+ = 497; HPLC tR = 3.15 min |
| 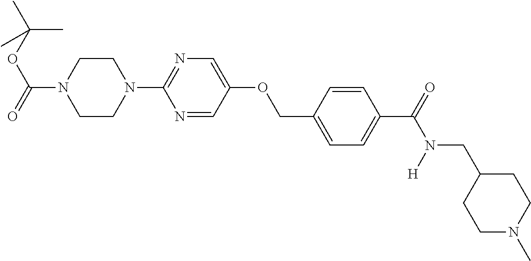 | 52 | tert-butyl 4-(5- (4-((1- methylpiper- idin-4- yl)methylcarba- moyl)benzyl- oxy)pyrimidin-2- yl)piperazine- 1-carboxylate | 1H NMR (DMSO d6) 1.4 (s, 9H), 1.55 (br, 1H), 2.15 (s, 3H), 2.8 (s, 6H), 3.4 (t, 4H), 3.65 (t, 4H), 3.8 (s, 1H), 5.15 (s, 2H), 7.35 (d, 2H), 7.45 (d, 2H) and 8.2 (s, 2H). | m/z (ES+) (M + H)+ = 526; HPLC tR = 3.24 min |
| 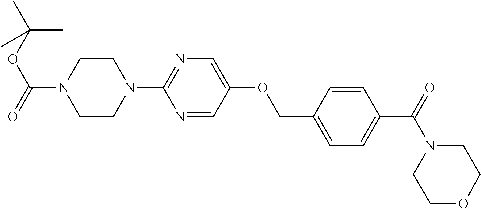 | 53 | tert-butyl 4-(5- (4-(morpholin- 4-yl)-1- carbonyl)benz- yloxy)pyrimi- din-2- yl)piperazine- 1-carboxylate | 1H NMR (DMSO d6) 1.45 (S, 9H), 3.4 (t, 4H), 3.5 (t, 4H), 3.6-3.7 (m, 8H), 5.15 (s, 2H), 7.4 (d, 2H), 7.5 (d, 2H) and 8.25 (s, 2H). | m/z (ES+) (M − Boc)+ = 384; HPLC tR = 3.10 min |

### Example 54

### Tert-butyl 4-(5-(4-(isopropylcarbamoyl)-3-methylbenzyloxy)pyrimidin-2-yl)piperazine-1-carboxylate

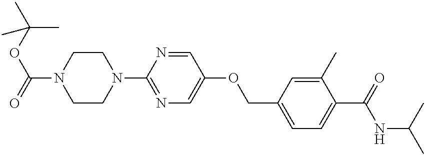

N-Ethyldiisopropylamine (0.211 mL, 1.21 mmol) was added to 4-((2-(4-(tert-butoxycarbonyl)piperazin-1-yl)pyrimidin-5-yloxy)methyl)-2-methylbenzoic acid (Intermediate 38) (130 mg, 0.30 mmol), propan-2-amine (35.9 mg, 0.61 mmol) and O-(7-azabenzotriazol-1-yl)-N,N,N′,N′-tetramethyluronium hexafluorophosphate (138 mg, 0.36 mmol) in DCM (4 mL) at ambient temperature. The resulting solution was stirred at ambient temperature for 20 hours. The reaction mixture was diluted with water (10 mL) and poured onto a phase separator. The organic layer was purified by flash silica chromatography, elution gradient O to 50% EtOAc in isohexane. Pure fractions were evaporated to dryness to afford tert-butyl 4-(5-(4-(isopropylcarbamoyl)-3-methylbenzyloxy)pyrimidin-2-yl)piperazine-1-carboxylate (109 mg, 77%) as a colourless solid. ¹H NMR (400.132 MHz, CDCl₃) 1.26 (6H, d), 1.48 (9H, s), 2.45 (3H, s), 3.48 (4H, t), 3.70 (4H, t), 4.22-4.33 (1H, m), 5.00 (2H, s), 5.51 (1H, d), 7.19-7.25 (2H, m), 7.34 (1H, d), 8.10 (2H, s). m/z (ES+) (M-Boc)=370.45; HPLC tR=3.31 min.

The following Example was prepared in a similar manner to Example 54, using Intermediate 38 and t-butylamine:

| Structure | Ex | Name | 1H NMR δ | MS |
|---|---|---|---|---|
| 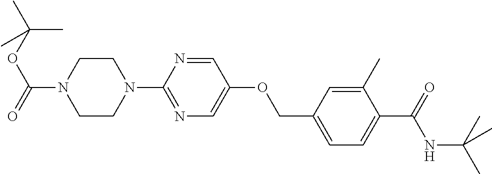 | 55 | tert-butyl 4-(5- (4-(tert- butylcarbamoyl)- 3- methylbenzyl- oxy)pyrimidin-2- yl)piperazine-1- carboxylate | 1H NMR (400.132 MHz, CDCl3) 1.46 (9H, s), 1.48 (9H, s), 2.44 (3H, s), 3.48 (4H, t), 3.70 (4H, t), 4.99 (2H, s), 5.51 (1H, s), 7.17- 7.23 (2H, m), 7.32 (1H, d), 8.10 (2H, s) | m/z (ES+) (M − Boc) = 384.47; HPLC tR = 3.47 min. |

### Example 56

Isopropyl 4-(5-(4-(methylsulfonyl)benzyloxy)pyrimidin-2-yl)piperazine-1-carboxylate

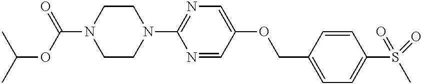

Isopropyl chloroformate, 1M solution in toluene (1.169 mL, 1.17 mmol) was added to 5-(4-(methylsulfonyl)benzyloxy)-2-(piperazin-1-yl)pyrimidine hydrochloride (Intermediate 35) (300 mg, 0.78 mmol) and triethylamine (1.304 mL, 9.35 mmol) in DCM (12.0 mL). The resulting solution was stirred at 20° C. for 18 hours. It was diluted with DCM (100 mL) and washed with 50% brine (50 mL), dried (sodium sulphate) and concentrated in vacuo to an off white solid (351 mg). The crude product was purified by flash silica chromatography, elution gradient 0 to 5% MeOH in DCM then recrystallised from DMSO:acetonitrile:water 7:2:1. This gave isopropyl 4-(5-(4-(methylsulfonyl)benzyloxy)-pyrimidin-2-yl)piperazine-1-carboxylate (219 mg, 65%) as a white solid. ¹H NMR (400 MHz, DMSO) 1.2 (d, 6H), 3.2 (s, 3H), 3.4 (t, 4H), 3.65 (t, 4H), 4.8 (m, 1H), 5.25 (s, 2H), 7.65 (d, 2H), 7.95 (d, 2H), 8.3 (s, 2H). m/z (ES+) (M+H)=435.16; HPLC tR=2.97 min

The following Examples were prepared in a similar manner to Example 56, using the appropriate piperazine (prepared by removal of the t-BOC group from the appropriate tert-butoxycarbonylpiperazine compound described herein using the method described to prepare Intermediate 33) and isopropyl chloroformate:

| Structure | Ex | Name | 1H NMR δ | MS |
|---|---|---|---|---|
| 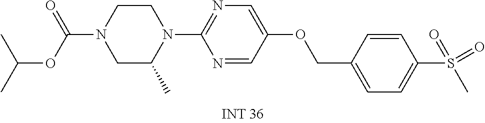 | 57 | (R)-isopropyl 3-methyl-4-(5- (4- (methylsulfonyl) benzyloxy) pyrimidin-2- yl)piperazine- 1-carboxylate | 1H NMR (400 MHz, DMSO) 1.0 (d, 3H), 1.2 (m, 6H), 2.9 (m, 1H), 3.0 (m, 2H), 3.2 (s, 3H), 3.8 (m, 1H), 3.95 (m, 1H), 4.25 (m, 1H), 4.7 (m, 1H), 4.8 (m, 1H), 5.2 (s, 2H), 7.7 (d, 2H), 7.9 (d, 2H), 8.3 (s, 2H). | m/z (ES+) (M + H)+ = 449.2; HPLC tR = 3.06 min |
| 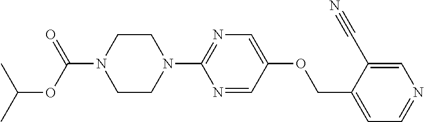 | 58 | isopropyl 4-(5- ((3- cyanopyridin- 4- yl)methoxy) pyrimidin-2- yl)piperazine- 1-carboxylate | 1H NMR (CDCl3) 1.2 (d, 6H), 3.45 (t, 4H), 3.7 (t, 4H), 4.9 (dt, 1H), 5.15 (s, 2H), 7.6 (d, 1H), 8.1 (s, 2H), 8.75 (d, 1H) and 8.8 (s, 1H). | m/z (ES+) (M + H)+ = 383; HPLC tR = 2.15 min |
| 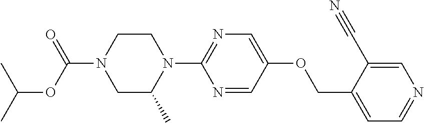 | 59 | (R)-isopropyl 4-(5-((3- cyanopyridin- 4- yl)methoxy) pyrimidin-2-yl)- 3- methylpiper- azine-1- carboxylate | 1H NMR (CDCl3) 1.1 (d, 3H), 1.2 (d, 6H), 2.9 (br, 1H), 3.05-3.15 (m, 2H), 3.85-4.2 (br, 2H), 4.3 (d, 1H), 4.75 (br, 1H), 4.9 (dt, 1H), 5.15 (s, 2H), 7.6 9d, 1H), 8.1 (s, 2H), 8.75 9d, 1H) and 8.8 (s, 1H). | m/z (ES+) (M + H)+ = 397; HPLC tR = 2.27 min |

### Example 60

### 1-Methylcyclopropyl 4-(5-(4-(methylsulfonyl)benzyloxy)pyrimidin-2-yl)piperazine-1-carboxylate

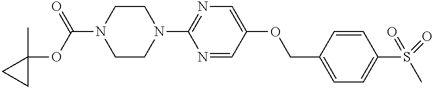

1-Methylcyclopropyl 4-nitrophenyl carbonate (296 mg, 1.25 mmol) in DCM (4mL) was added to 5-(4-(methylsulfonyl)benzyloxy)-2-(piperazin-1-yl)pyrimidine hydrochloride (Intermediate 35) (400 mg, 1.04 mmol) and triethylamine (0.290 mL, 2.08 mmol) in DCM (20 mL). The resulting suspension was stirred at 20° C. for 18 hours. The reaction mixture was diluted with DCM (100 mL) and washed with 50% brine (50 mL), dried (sodium sulphate), concentrated in vacuo and adsorbed onto silica. The crude product was purified by flash silica chromatography, elution gradient 0 to 100% EtOAc in isohexane. It was adsorbed onto silica and the mixture was re-purified by flash silica chromatography, elution gradient 0 to 30% EtOAc in DCM. Pure fractions were evaporated to dryness then recrystallised from DMSO:acetonitrile:water 7:2:1. This gave 1-methylcyclopropyl 4-(5-(4-(methylsulfonyl)benzyloxy)pyrimidin-2-yl)piperazine-1-carboxylate (171 mg, 37%) as a white solid. ¹H NMR (400 MHz, DMSO) 0.6 (t, 2H), 0.8 (t, 2H), 1.5 (t, 3H), 3.2 (s, 3H), 3.4 (m, 4H), 3.65 (t, 4H), 5.2 (s, 2H), 7.7 (d, 2H), 7.95 (d, 2H), 8.3 (s, 2H).

m/z (ES+) (M+H)+=447.20; HPLC tR=2.97 min.

The following Examples were prepared in a similar manner to Example 60, using Intermediate 35 and the appropriate 4-nitrophenyl carbonate:

| Structure | Ex | Name | 1H NMR δ | MS |
|---|---|---|---|---|
| 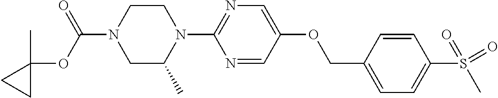 | 61 | (R)-1- methylcyclo- propyl 3-methyl- 4-(5-(4- (methylsulfonyl) benzyloxy) pyrimidin-2- yl)piperazine- 1-carboxylate | 1H NMR (400 MHz, DMSO) 0.7 (m, 2H), 0.85 (m, 2H), 1.1 (d, 3H), 1.55 (s, 3H), 2.9 (m, 1H), 3.1 (m, 2H), 3.25 (s, 3H), 3.8-4.1 (m, 2H), 4.3 (m, 1H), 4.75 (m, 1H), 5.3 (s, 2H), 7.75 (d, 2H), 8.0 (d, 2H), 8.35 (s, 2H). | m/z (ES−) (M − H)− = 459.55; HPLC tR = 3.06 min |
| 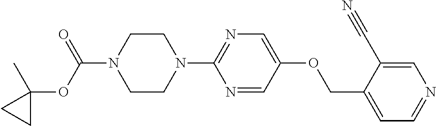 | 62 | 1- methylcyclo- propyl 4-(5-((3- cyanopyridin- 4- yl)methoxy) pyrimidin-2- yl)piperazine- 1-carboxylate | 1H NMR (400 MHz, DMSO) 0.6 (t, 2H), 0.8 (t, 2H), 1.5 (s, 3H), 3.4 (m, 4H), 3.65 (t, 4H), 5.4 (s, 2H), 7.7 (d, 1H), 8.3 (s, 2H), 8.85 (d, 1H), 9.05 (s, 1H). | m/z (ES+) (M + H)+ = 395.30; HPLC tR = 2.21 min |

### Example 63

### Cyclobutyl 4-(5-(4-(methylsulfonyl)benzyloxy)pyrimidin-2-yl)piperazine-1-carboxylate

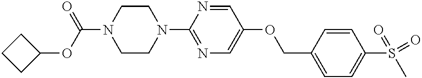

Triethylamine (0.362 mL, 2.60 mmol) was added to 5-(4-(methylsulfonyl)benzyloxy)-2-(piperazin-1-yl)pyrimidine hydrochloride (Intermediate 35) (0.25 g, 0.65 mmol) and cyclobutyl 2,5-dioxopyrrolidin-1-yl carbonate (0.180 g, 0.84 mmol) in DCM (15 mL) at 20° C. under nitrogen. The resulting solution was stiffed at 20° C. for 24 hours. The reaction mixture was diluted with DCM (20 mL), and washed sequentially with water (25 mL) and saturated brine (50 mL). The organic layer was dried over Na₂SO₄, filtered and evaporated to afford crude product. The crude product was purified by flash silica chromatography, elution gradient 1 to 5% MeOH in DCM. Pure fractions were evaporated to dryness to afford cyclobutyl 4-(5-(4-(methylsulfonyl)benzyloxy)pyrimidin-2-yl)piperazine-1-carboxylate (0.243 g, 84%) as a white solid. ¹H NMR (400.132 MHz, CDCl₃) δ1.56-1.67 (1H, m), 1.73-1.83 (1H, m), 2.02-2.14 (2H, m), 2.31-2.40 (2H, m), 3.06 (3H, s), 3.51-3.55 (4H, m), 3.71-3.74 (4H, m), 4.97 (1H, quintet), 5.12 (2H, s), 7.62 (2H, d), 7.98 (2H, d), 8.13 (2H, s). m/z (ES+) (M+H)+=447; HPLC tR=3.06 min

The following Examples were prepared in a similar manner to Example 63, using the Intermediate stated (or alternatively the appropriate piperazine was prepared by removal of the t-BOC group from the appropriate tert-butoxycarbonylpiperazine compound described herein using the method described to prepare Intermediate 33) and the appropriate 2,5-dioxopyrrolidin-1-yl carbonate:

| Structure | Ex | Name | 1H NMR δ | MS |
|---|---|---|---|---|
| 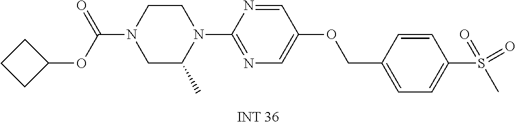 | 64 | (R)- cyclobutyl 3-methyl-4- (5-(4- (methylsulf- onyl)benzyl- oxy)pyrimi- din-2- yl)piperazine- 1- carboxylate | 1H NMR (400.13 MHz, DMSO-d6) 1.03 (3H, d), 1.52- 1.61 (1H, m), 1.67-1.75 (1H, m), 1.91-2.06 (2H, m), 2.21- 2.32 (2H, m), 2.90-3.07 (3H, m), 3.21 (3H, s), 3.78-3.84 (1H, m), 3.92-3.99 (1H, m), 4.23-4.27 (1H, m), 4.65-4.73 (1H, m), 4.82-4.89 (1H, m), 5.23 (2H, s), 7.69 (2H, d), 7.93- 7.95 (2H, m), 8.29 (2H, s) | m/z (ES+) (M + H)+ = 461.24; HPLC tR = 3.14 min. |
| 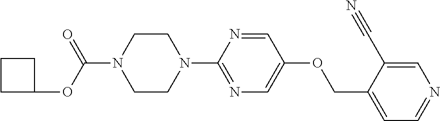 | 65 | cyclobutyl 4-(5-((3- cyanopyri- din-4- yl)methoxy) pyrimidin- 2- yl)piperazine- 1- carboxylate | 1H NMR (400.13 MHz, DMSO-d6) 1.49-1.51 (1H, m), 1.66-1.76 (1H, m), 1.94- 2.06 (2H, m), 2.20-2.29 (2H, m), 3.42 (4H, s), 3.64 (2H, tsp), 3.65 (2H, m), 4.85 (1H, td), 5.34 (2H, s), 7.73 (1H, dd), 8.33 (2H, s), 8.87 (1H, d), 9.05 (1H, d) | m/z (ES+) (M + H)+ = 395.36; HPLC tR = 2.25 min. |

### Example 66

### 1,1,1-Trifluoropropan-2-yl 4-(5-(4-(methylsulfonyl)benzyloxy)pyrimidin-2-yl)piperazine-1-carboxylate

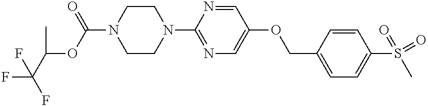

Triethylamine (0.290 mL, 2.08 mmol) was added to 5-(4-(methylsulfonyl)benzyloxy)-2-(piperazin-1-yl)pyrimidine hydrochloride (Intermediate 35) (200 mg, 0.52 mmol) and phenyl 1,1,1-trifluoropropan-2-yl carbonate (Intermediate 39) (243 mg, 1.04 mmol) in DCM (15 mL) under nitrogen. The resulting solution was stirred at 20° C. for 18 hours. Only 5% product was evident so solvent swapped for CHCl3 (10 mL) and heated at 75° C. for 16 hours. Reaction was diluted with DCM (10 mL) and washed with water (10 mL) and concentrated in vacuo to a pale yellow gum. The crude product was purified by flash silica chromatography, elution gradient 0 to 80% EtOAc in isohexane. Pure fractions were is evaporated to dryness to afford 1,1,1-trifluoropropan-2-yl 4-(5-(4-(methylsulfonyl)benzyloxy)pyrimidin-2-yl)piperazine-1-carboxylate (70.0 mg, 28%) as a colourless gum. ¹H NMR (400.13 MHz, DMSO-d6) 1.37 (3H, d), 3.21 (3H, s), 3.43-3.50 (4H, m), 3.60-3.70 (4H, m), 5.25 (2H, s), 5.30-5.38 (1H, m), 7.69 (2H, d), 7.94 (2H, dt), 8.30 (2H, s). m/z (ES+) (M+H)+=489.26; HPLC tR=3.13 min.

The following Examples were prepared in a similar manner to Example 66, using the Intermediate stated (or alternatively the appropriate piperazine was prepared by removal of the t-BOC group from the appropriate tert-butoxycarbonylpiperazine compound described herein using the method described to prepare Intermediate 33) and the appropriate carbonate:

| Structure | Ex | Name | 1H NMR δ | MS |
|---|---|---|---|---|
| 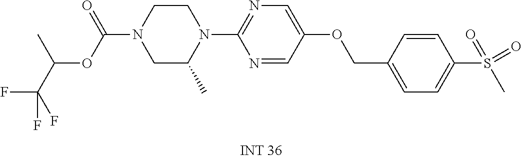 | 67 | (3R)-1,1,1- trifluoropropan- 2-yl 3-methyl-4- (5-(4- (methylsulfonyl) benzyloxy)pyrim- idin-2- yl)piperazine-1- carboxylate | 1H NMR (400 MHz, DMSO) 1.05 (d, 3H), 1.4 (d, 3H), 3.05 (m, 2H), 3.2 (m, 4H), 3.8 (m, 1H), 4.0 (m, 1H), 4.3 (m, 1H), 4.75 (m, 1H), 5.25 (s, 2H), 5.4 (m, 1H), 7.7 (d, 2H), 7.95 (d, 2H), 8.3 (s, 2H). | m/z (ES+) (M + H)+ = 4503.11; HPLC tR = 3.21 min. |
| 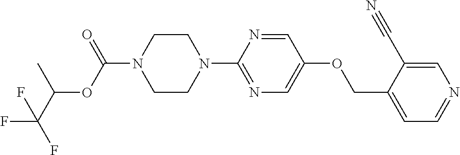 | 68 | 1,1,1- trifluoropropan- 2-yl 4-(5-((3- cyanopyridin-4- yl)methoxy)pyrim- idin-2- yl)piperazine-1- carboxylate | 1H NMR (400.13 MHz, DMSO-d6) 1.37 (3H, d), 3.46-3.48 (5H, m), 3.67- 3.69 (5H, m), 5.31- 5.38 (3H, m), 7.72-7.74 (1H, m), 8.34 (2H, s), 8.87 (1H, d), 9.05 (1H, d) | m/z (ES+) (M + H)+ = 437.19; HPLC tR = 2.47 min. |

### Example 70

### 2-Cyanopropan-2-yl 4-(5-(4-(methylsulfonyl)benzyloxy)pyrimidin-2-yl)piperazine-1-carboxylate

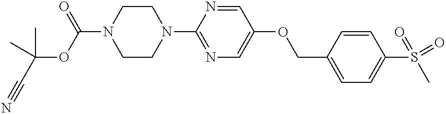

Triethylamine (0.290 mL, 2.08 mmol) was added to 5-(4-(methylsulfonyl)benzyloxy)-2-(piperazin-1-yl)pyrimidine hydrochloride (Intermediate 35) (200 mg, 0.52 mmol) and 2-cyanopropan-2-ylphenyl carbonate (213 mg, 1.04 mmol) in DCM (15 mL) under nitrogen. The resulting solution was stiffed at 20° C. for 18 hours. Only 5% product was evident so solvent swapped for CHCl₃ (10 mL) and heated at 75° C. for 16 hours. Reaction was diluted with DCM (10 mL) and washed with water (10 mL) and concentrated in vacuo to a pale yellow gum. The crude product was purified by flash silica chromatography, elution gradient 0 to 80% EtOAc in isohexane. Pure fractions were evaporated to dryness to afford 2-cyanopropan-2-yl 4-(5-(4-(methylsulfonyl)benzyloxy)pyrimidin-2-yl)piperazine-1-carboxylate (45.0 mg, 19%) as a colourless gum. ¹H NMR (400.13 MHz, DMSO-d6) 1.72 (6H, s), 3.30 (4H, s), 3.43-3.50 (4H, m), 3.60-3.70 (4H, m), 5.25 (2H, s), 7.69 (2H, d), 7.94 (2H, dt), 8.29 (2H, s). m/z (ES+) (M+H)+=460.29; HPLC tR=2.73 min.

The following Examples were prepared in a similar manner to Example 70, using the appropriate piperazine (prepared by removal of the t-BOC group from the appropriate tert-butoxycarbonylpiperazine compound described herein using the method described to prepare Intermediate 33) and the appropriate carbonate:

| Structure | Ex | Name | 1H NMR δ | MS |
|---|---|---|---|---|
| 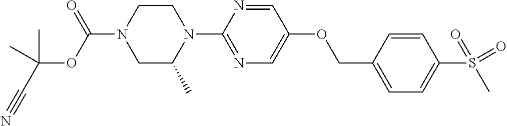 | 71 | (R)-2- cyanopropan- 2-yl 3-methyl- 4-(5-(4- (methylsulfonyl) benzyloxy) pyrimidin-2- yl)piperazine- 1-carboxylate | 1H NMR (400.13 MHz, DMSO-d6) δ 1.04 (3H, d), 1.72 (6H, s), 2.94-3.21 (6H, m), 3.74-4.00 (2H, m), 4.21-4.32 (1H, m), 4.65-4.71 (1H, m), 5.24 (2H, s), 7.70 (2H, d), 7.93- 7.96 (2H, m), 8.30 (2H, d) | m/z (ES+) (M + H)+ = 474.20; HPLC tR = 2.31 min. |
| 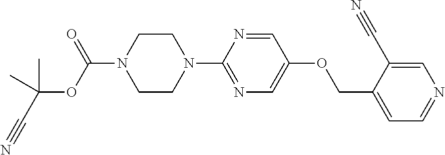 | 72 | 2- cyanopropan- 2-yl 4-(5-((3- cyanopyridin- 4- yl)methoxy) pyrimidin-2- yl)piperazine- 1-carboxylate | 1H NMR (400.13 MHz, DMSO-d6) 1.72 (6H, s), 3.43-3.46 (4H, m), 3.64- 3.71 (4H, m), 5.34 (2H, s), 7.72-7.74 (1H, m), 8.34 (2H, s), 8.87 (1H, d), 9.05 (1H, d) | m/z (ES+) (M + H)+ = 408.34; HPLC tR = 2.11 min. |
| 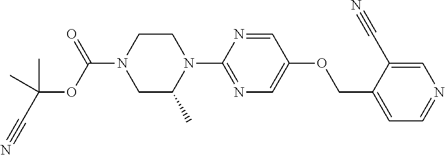 | 73 | (R)-2- cyanopropan- 2-yl 4-(5-((3- cyanopyridin- 4- yl)methoxy) pyrimidin-2-yl)- 3- methylpiper- azine-1- carboxylate | 1H NMR (400.13 MHz, CDCl3) 1.18 (3H, d), 1.80 (6H, s), 2.95-3.07 (1H, m), 3.16-3.23 (2H, m), 3.80 (1H, d), 3.97 (1H, q), 4.20 (1H, d), 4.41 (1H, t), 4.83 (1H, s), 5.21 (2H, s), 7.65- 7.66 (1H, m), 8.19 (2H, s), 8.85 (1H, d), 8.91 (1H, s) | m/z (ES+) (M + H)+ = 422; HPLC tR = 2.16 min. |

### Example 75

### Oxetan-3-yl 4-(5-(4-(methylsulfonyl)benzyloxy)pyrimidin-2-yl)piperazine-1-carboxylate

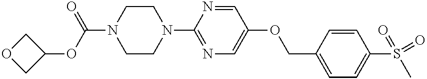

Triethylamine (0.362 mL, 2.60 mmol) was added to 5-(4-(methylsulfonyl)benzyloxy)-2-(piperazin-1-yl)pyrimidine hydrochloride (Intermediate 35) (0.25 g, 0.65 mmol) and 2,5-dioxopyrrolidin-1-yl oxetan-3-yl carbonate (0.260 g, 0.84 mmol) in DCM (15 mL) at 20° C. under nitrogen. The resulting solution was stiffed at 20° C. for 4 hours. The reaction mixture was diluted with DCM (20 mL), and washed sequentially with water (25 mL) and saturated brine (50 mL). The organic layer was dried over Na₂SO₄, filtered and evaporated to afford crude product. The crude product was purified by flash silica chromatography, elution gradient 1 to 5% MeOH in DCM. Pure fractions were evaporated to dryness to afford oxetan-3-yl 4-(5-(4-(methylsulfonyl)benzyloxy)pyrimidin-2-yl)piperazine-1-carboxylate (0.192 g, 66%) as a white solid. ¹H NMR (400.132 MHz, CDCl₃) 3.07 (3H, s), 3.51-3.59 (4H, m), 3.74-3.79 (4H, m), 4.66-4.70 (2H, m), 4.90 (2H, t), 5.13 (2H, s), 5.43 (1H, quintet), 7.62 (2H, d), 7.97 (2H, d), 8.14 (2H, s). m/z (ES+) (M−H)−=447; HPLC tR=2.49 min.

The following Examples were prepared in a similar manner to Example 75, using the appropriate piperazine prepared as described previously and 2,5-dioxopyrrolidin-1-yl oxetan-3-yl carbonate:

| Structure | Ex | Name | 1H NMR δ | MS |
|---|---|---|---|---|
| 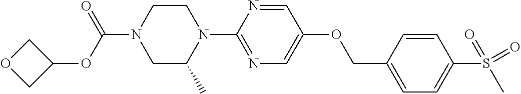 | 76 | (R)-oxetan-3- yl 3-methyl-4- (5-(4- (methylsulfonyl) benzyloxy) pyrimidin-2- yl)piperazine- 1-carboxylate | 1H NMR (400.13 MHz, DMSO-d6) 1.05 (3H, d), 2.82-3.15 (3H, m), 3.21 (3H, s), 3.76-4.06 (2H, m), 4.27 (1H, dd), 4.46-4.54 (2H, m), 4.68-4.80 (3H, m), 5.24 (2H, s), 5.32 (1H, ddd), 7.70 (2H, d), 7.95 (2H, dt), 8.30 (2H, s) | m/z (ES+) (M + H)+ = 463.3; HPLC tR = 2.56 min. |
| 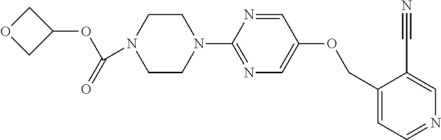 | 77 | oxetan-3-yl 4- (5-((3- cyanopyridin- 4- yl)methoxy) pyrimidin-2- yl)piperazine- 1-carboxylate | 1H NMR (400.13 MHz, DMSO-d6) 3.46-3.50 (4H, m), 3.67-3.69 (4H, m), 4.49-4.52 (2H, m), 4.74- 4.78 (2H, m), 5.28-5.32 (1H, m), 5.29-5.34 (2H, m), 7.72-7.74 (1H, m), 8.34 (2H, s), 8.87 (1H, d), 9.05 (1H, d) | m/z (ES+) (M + H)+ = 397.4; HPLC tR = 1.66 min. |

### Example 78

### (3R)-Tetrahydrofuran-3-yl 3-methyl-4-(5-(4-(methylsulfonyl)benzyloxy)pyrimidin-2-yl)piperazine-1-carboxylate

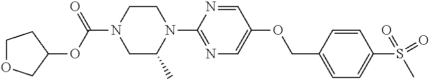

Triethylamine (0.288 mL, 2.07 mmol) was added dropwise to 2,5-dioxopyrrolidin-1-yl tetrahydrofuran-3-yl carbonate (Intermediate 36) (0.154 g, 0.67 mmol) and (R)-2-(2-methylpiperazin-1-yl)-5-(4-(methylsulfonyl)benzyloxy)pyrimidine hydrochloride (0.206 g, 0.52 mmol) in DCM (15 mL) at 20° C. over a period of 1 minute under nitrogen. The io resulting solution was stirred at 20° C. for 19 hours. The reaction mixture was diluted with DCM (50 mL), and washed with 2M K₂CO₃ aq (20 mL). The organic layer was dried over MgSO₄, filtered and evaporated to afford crude product. The crude product was purified by flash silica chromatography (40 g column), elution gradient 0 to 100% EtOAc in DCM. Pure fractions were evaporated to dryness to afford (3R)-tetrahydrofuran-3-yl 3-methyl-4-(5-(4-(methylsulfonyl)benzyloxy)pyrimidin-2-yl)piperazine-1-carboxylate (0.191 g, 78%) as a white solid. ¹H NMR (400.13 MHz, DMSO-d6) 1.02 (3H, d), 1.86-1.95 (1H, m), 2.06-2.16 (1H, m), 3.01-3.11 (3H, m), 3.21 (3H, s), 3.66-3.83 (5H, m), 3.90-4.03 (1H, m), 4.22-4.28 (1H, m), 4.65-4.73 (1H, m), 5.15-5.17 (1H, m), 5.23 (2H, s), 7.69 (2H, d), 7.93-7.96 (2H, m), 8.29 (2H, s). m/z (ES+) (M+H)+=477.22; HPLC tR=2.66 min.

### Example 79

### 3-Methyloxetan-3-yl 4-(5-(4-(methylsulfonyl)benzyloxy)pyrimidin-2-yl)piperazine-1-carboxylate

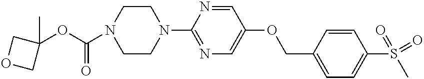

4-Fluorophenyl 3-methyloxetan-3-yl carbonate (0.147 g, 0.65 mmol) was added to 5-(4-(methylsulfonyl)benzyloxy)-2-(piperazin-1-yl)pyrimidine hydrochloride (Intermediate 35) (0.25 g, 0.65 mmol) and triethylamine (0.272 mL, 1.95 mmol) in CHCl₃ (10 mL) at 20° C. under nitrogen. The reaction was heated at 120° C. for 18 hours by which time the solvent had evaporated and a dark gum remained. The reaction mixture was diluted with DCM (25 mL), and washed sequentially with water (25 mL) and saturated brine (25 mL). The organic layer was dried over Na₂SO₄, filtered and evaporated to afford crude product. The crude product was purified by flash silica chromatography, elution gradient 1 to 5% MeOH in DCM. Pure fractions were evaporated to dryness to afford 3-methyloxetan-3-yl 4-(5-(4-(methylsulfonyl)benzyloxy)pyrimidin-2-yl)piperazine-1-carboxylate (0.032 g, 11%) as a white solid. ¹H NMR (400.132 MHz, CDCl₃) 1.74 (3H, s), 3.06 (3H, s), 3.49-3.56 (4H, m), 3.72-3.77 (4H, m), 4.51 (2H, d), 4.80 (2H, d), 5.12 (2H, s), 7.62 (2H, d), 7.98 (2H, d), 8.13 (2H, s). m/z (ES+) (M+H)+=463; HPLC tR=1.94 min.

The following Examples were prepared in a similar manner to Example 79, using an Intermediate piperazine prepared as described previously and an appropriate carbonate:

| Structure | Ex | Name | 1H NMR δ | MS |
|---|---|---|---|---|
| 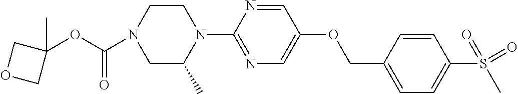 | 80 | (R)-3- methyloxetan-3- yl 3-methyl-4- (5-(4- (methylsulfonyl) benzyloxy)pyrimi- din-2- yl)piperazine-1- carboxylate | 1H NMR (400.132 MHz, CDCl3) 1.17 (3H, d), 1.75 (3H, s), 2.88-2.98 (1H, m), 3.07 (3H, s), 3.14-3.26 (2H, m), 3.82-4.19 (2H, m), 4.30-4.40 (1H, m), 4.50 (2H, d), 4.80-4.82 (3H, m), 5.12 (2H, s), 7.62 (2H, d), 7.98 (2H, d), 8.13 (2H, s) | m/z (ES+) (M + H)+ = 477; HPLC tR = 2.03 min. |
| 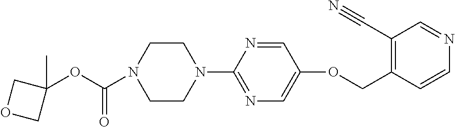 | 81 | 3-methyloxetan- 3-yl 4-(5-((3- cyanopyridin-4- yl)methoxy)pyrimi- din-2- yl)piperazine-1- carboxylate | 1H NMR (400.132 MHz, CDCl3) 1.76 (3H, s), 3.48- 3.56 (4H, m), 3.74-3.81 (4H, m), 4.51 (2H, d), 4.81 (2H, d), 5.21 (2H, s), 7.65 (1H, d), 8.19 (2H, s), 8.85 (1H, d), 8.91 (1H, s) | m/z (ES+) (M + H)+ = 411; HPLC tR = 1.80 min. |

### Example 82

### (R)-Tetrahydro-2H-pyran-4-yl 4-(5-((3-cyanopyridin-4-yl)methoxy)pyrimidin-2-yl)-3-methylpiperazine-1-carboxylate

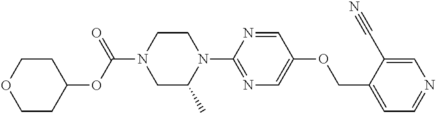

Triethylamine (0.485 mL, 3.48 mmol) was added to (R)-4-((2-(2-methylpiperazin-1-yl)pyrimidin-5-yloxy)methyl)nicotinonitrile (270 mg, 0.87 mmol) and 2,5-dioxopyrrolidin-1-yl tetrahydro-2H-pyran-4-yl carbonate (275 mg, 1.13 mmol) in DCM (20 mL) at 20° C. under nitrogen. The resulting solution was stiffed at 20° C. for 4 hours. The reaction mixture was diluted with DCM (20 mL), and washed sequentially with water (25 mL) and saturated brine (50 mL). The organic layer was dried over Na₂SO₄, filtered and evaporated to afford crude product. The crude product was purified by flash silica chromatography, elution gradient 1 to 5% MeOH in DCM. Pure fractions were evaporated to dryness to afford (R)-tetrahydro-2H-pyran-4-yl 4-(5-((3-cyanopyridin-4-yl)methoxy)pyrimidin-2-yl)-3-methylpiperazine-1-carboxylate (299 mg, 78%) as a white solid after trituration with diethyl ether. ¹H NMR (400.132 MHz, CDCl₃) 1.17 (3H, d), 1.63-1.77 (2H, m), 1.90-2.02 (2H, m), 2.87-3.10 (1H, m), 3.11-3.24 (2H, m), 3.51-3.62 (2H, m), 3.86-4.03 (3H, m), 4.04-4.26 (1H, m), 4.35-4.43 (1H, m), 4.77-4.86 (1H, m), 4.88-4.97 (1H, m), 5.21 (2H, s), 7.65 (1H, d), 8.18 (2H, s), 8.85 (1H, d), 8.91 (1H, s). m/z (ES+) (M+H)+=439.50; HPLC tR=1.95 min.

### Example 83

### Tert-butyl 4-(5-(4-(ethylsulfonyl)benzyloxy)pyrimidin-2-yl)piperazine-1-carboxylate

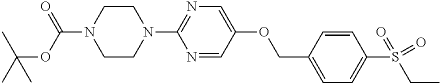

To a stirred solution of tert-butyl 4-(5-(4-iodobenzyloxy)pyrimidin-2-yl)piperazine-1-carboxylate (496 mg, 1.0 mmol), (trifluoromethylsulfonyloxy)copper (42.5 mg, 0.20 mmol) and N¹,N²-dimethylethane-1,2-diamine (35.3 mg, 0.40 mmol) in DMSO (10.0 mL) at ambient temperature under an atmosphere of nitrogen was added sodium ethanesulfinate (581 mg, 5.00 mmol). The mixture was heated at 120° C. for 1 hour, cooled to ambient temperature, partitioned between ethyl acetate (150 mL) and water (50 mL), washed with water (1×50 mL), brine (2×50 mL), dried (MgSO₄) and evaporated in vacuo to a residue which was chromatographed on silica with 50% ethyl acetate in isohexane as eluant to give a solid which was crystallised from ethyl acetate/isohexane to give tert-butyl 4-(5-(4-(ethylsulfonyl)benzyloxy)pyrimidin-2-yl)piperazine-1-carboxylate (420 mg, 91%). ¹H NMR (CDCl₃). 1.2 (t, 3H), 1.4 (s, 9H), 3.05 (q, 2H), 3.4 (t, 4H), 3.65 (t, 4H), 5.0 (s, 2H), 7.5 (d, 2H), 7.85 (d, 2H) and 8.05 (s, 2H). m/z (ES+) (M-tBu)+=407; HPLC tR =2.56 min

The following Example was prepared in a similar manner to Example 83, using tert-butyl 4-(5-(4-iodobenzyloxy)pyrimidin-2-yl)piperazine-1-carboxylate and sodium cyclopropylsulfinate:

| Structure | Ex | Name | 1H NMR δ | MS |
|---|---|---|---|---|
| 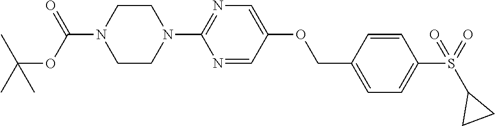 | 84 | Tert-butyl 4- (5-(4- (cyclopropyl- sulfonyl) benzyloxy) pyrimidin-2- yl)pipcrazinc- 1-carboxylate | 1H NMR(CDCl3) 1.0 (dt, 2H), 1.3 (dt, 2H), 1.4 (s, 9H), 2.4 (dt, 1H), 3.4 (t, 4H), 3.65 (dt, 4H), 5.05 (s, 2H), 7.5 (d, 2H), 7.85 (dt, 2H0 and 8.05 (s, 2H). | m/z (ES+) (M + H)+ = 475; HPLC tR = 2.64 min |

### Example 85

### 4-((2-(4-(5-Isopropyl-1,2,4-oxadiazol-3-yl)piperazin-1-yl)pyrimidin-5-yloxy)methyl)nicotinonitrile

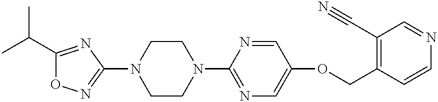

3-(4-(5-((3-Bromopyridin-4-yl)methoxy)pyrimidin-2-yl)piperazin-1-yl)-5-isopropyl-1,2,4-oxadiazole (Intermediate 41) (0.14 g, 0.30 mmol), zinc cyanide (0.029 g, 0.24 mmol), tris(dibenzylideneacetone)dipalladium(0) (0.011 g, 0.01 mmol) and 4,5-bis(diphenyl-phosphino)-9,9-dimethylxanthene (0.014 g, 0.02 mmol) (xantphos) were suspended in DMF (2 mL) and sealed into a microwave tube (evacuated and purged with nitrogen). The reaction was heated to 130° C. for 60 minutes in the microwave reactor and cooled to RT. The reaction mixture was filtered through celite. The reaction mixture was diluted with EtOAc (100 mL), and washed sequentially with water (100 mL) and saturated brine (100 mL). The organic layer was dried over Na₂SO₄, filtered and evaporated to afford crude product. The crude product was purified by flash silica chromatography, elution gradient 10 to 50% EtOAc in DCM. Pure fractions were evaporated to dryness to afford 4-((2-(4-(5-isopropyl-1,2,4-oxadiazol-3-yl)piperazin-1-yl)pyrimidin-5-yloxy)methyl)nicotinonitrile (0.032 g, 26%) as a white solid. ¹H NMR (400.132 MHz, CDCl₃) 1.37 (6H, d), 3.09 (1H, septet), 3.50-3.54 (4H, m), 3.84-3.89 (4H, m), 5.22 (2H, s), 7.66 (1H, d), 8.19 (2H, s), 8.85 (1H, d), 8.91 (1H, s). m/z (ES+) (M+H)+=407; HPLC tR=2.36 min.

### Example 86

### (R)-4-((2-(4-(5-isopropyl-1,2,4-oxadiazol-3-yl)-2-methylpiperazin-1-yl)pyrimidin-5-yloxy)methyl)nicotinonitrile

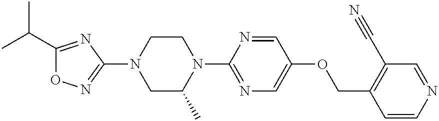

Tris(dibenzylideneacetone)dipalladium(0) (0.332 g, 0.36 mmol) and 4,5-bis(diphenylphosphino)-9,9-dimethylxanthene (0.420 g, 0.73 mmol) were stirred in DMA (25 ml) which had been thoroughly degassed. The catalyst mixture was heated to 50° C. for 30 mins to pre-form the catalyst. In a separate pot (R)-3-(4-(5-((3-bromopyridin-4-yl)methoxy)pyrimidin-2-yl)-3-methylpiperazin-1-yl)-5-isopropyl-1,2,4-oxadiazole (4.3 g, 9.06 mmol) (Intermediate 42), Zn powder (0.059 g, 0.90 mmol), and zinc cyanide (0.852 g, 7.25 mmol) were stiffed in another 25 ml of DMA and water (5 ml), which had again been degassed, and was kept under nitrogen. The mixture containing the starting material and zinc cyanide was added in aliquots to the catalyst mixture. Once addition was complete the reaction mixture was heated to 60° C. for 6 hrs. Another set of equivs of the zinc cyanide is and the catalysts were then added and left at 60° C. overnight. The reaction was filtered through celite, and washed through with ethyl acetate and water. The filtrate was diluted with EtOAc (750 ml), and washed sequentially with water (2×400 ml) and saturated brine (100 mL). The organic layer was evaporated to afford crude product which was purified by flash silica chromatography, elution gradient 0 to 80% EtOAc in isohexane. Pure fractions were evaporated to dryness to afford a yellow solid, which was triturated with a small amount of diethyl ether yielding (R)-4-((2-(4-(5-isopropyl-1,2,4-oxadiazol-3-yl)-2-methylpiperazin-1-yl)pyrimidin-5-yloxy)methyl)nicotinonitrile (2.70 g, 71%) as a pale yellow solid. ¹H NMR (400.13 MHz, DMSO-d6) 1.12 (3H, d), 1.26 (6H, d), 2.91-3.00 (1H, m), 3.06-3.22 (3H, m), 3.72 (1H, dt), 3.86 (1H, dt), 4.36 (1H, q), 4.77-4.85 (1H, m), 5.34 (2H, s), 7.74 (1H, dd), 8.34 (2H, s), 8.87 (1H, d), 9.05 (1H, d). m/z (ES+) (M+H)+=421.45; HPLC tR=2.5 min. Alternatively the product was purified by stiffing it in methanol (approximately 10 rel vols) at ambient temperature for 4 days then collecting the solid by filtration and drying it under vacuum.

### Example 87

### 5-Isopropyl-3-(4-(5-(4-(methylsulfonyl)benzyloxy)pyrimidin-2-yl)piperazin-1-yl)-1,2,4-oxadiazole

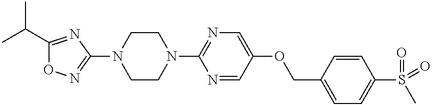

(E)-N-((Hydroxyimino)(4-(5-(4-(methylsulfonyl)benzyloxy)pyrimidin-2-yl)piperazin-1-yl)methyl)isobutyramide (Intermediate 45) (0.424 g, 0.89 mmol) was suspended in toluene (80 mL) and stirred at 120° C. for 30 minutes. All volatiles were removed under reduced pressure and the residue was diluted with DCM (75 mL), and washed with 2M K₂CO₃ aq. (20 mL). The organic layer was dried over MgSO₄, filtered and evaporated to afford crude product. The crude product was purified by flash silica chromatography, elution gradient 0 to 100% EtOAc in DCM. The fastest spot fractions were evaporated to dryness to afford 5-isopropyl-3-(4-(5-(4-(methylsulfonyl)benzyloxy)pyrimidin-2-yl)piperazin-1-yl)-1,2,4-oxadiazole (0.230 g, 56%). ¹H NMR (400.13 MHz, DMSO-d6) 1.27 (6H, d), 3.08-3.15 (1H, m), 3.21 (3H, s), 3.37-3.40 (4H, m), 3.72-3.74 (4H, m), 5.25 (2H, s), 7.70 (2H, d), 7.93-7.95 (2H, m), 8.30 (2H, s). m/z (ES+) (M+H)+=459.42; HPLC tR=2.46 min.

The following Examples were prepared in a similar manner to Example 87, using the intermediates listed below:

| structure and intermediates | Ex | Name | 1H NMR δ | MS |
|---|---|---|---|---|
| 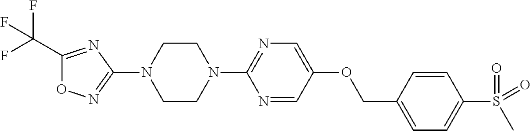 INT 46 | 88 | 3-(4-(5-(4- (methylsulfonyl) bcnzyloxy) pyrimidin-2-yl) pipcrazin-1-yl)-5- (trifluoromcthyl)- 1,2,4-oxadiazole | 1H NMR (400.13 MHz, CDCl3) 3.06 (3H, s), 3.57- 3.59 (4H, m), 3.85-3.89 (4H, m), 5.13 (2H, s), 7.62 (2H, d), 7.96- 8.00 (2H, m), 8.15 (2H, s) | m/z (ES+) (M + McCN)+ = 526.22; HPLC tR = 2.77 min. |
| 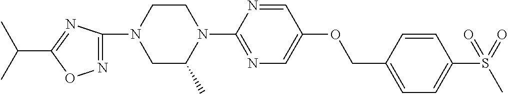 INT 47 | 89 | (R)-5-isopropyl- 3-(3-methy]-4-(5- (4-(mcthylsulfonyl) bcnzyloxy) pyrimidin- 2-yl)pipcrazin- 1-yl)-1,2,4- oxadiazole | 1H NMR (400.13 MHz, CDCl3) 1.24 (3H, d), 1.35 (3H, d), 2.99-3.13 (5H, m), 3.21 (1H, dd), 3.30 (1H, ddd), 3.84 (1H, dt), 4.00 (1H, d5), 4.44 (1H, dq), 4.85-4.92 (1H, m), 5.12 (2H, s), 7.62 (2H, d), 7.98 (2H, dt), 8.14 (2H, s) | m/z (ES+) (M + H)+ = 473.47; HPLC tR = 2.57 min. |
| 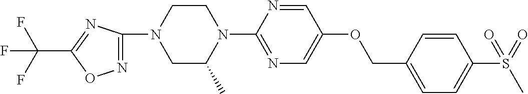 INT 48 | 90 | (R)-3-(3-methyl- 4-(5-(4- (mcthylsulfonyl) bcnzyloxy) pyrimidin-2-yl) pipcrazin-1-yl)- 5-(trifluoromcthyl)- 1,2,4-oxadiazole | 1H NMR (400.13 MHz, DMSO-d6) 1.12 (3H, d), 3.07-3.33 (6H, m), 3.75 (1H, dt), 3.86-3.91 (1H, m), 4.36-4.42 (1H, m), 4.80- 4.87 (1H, m), 5.25 (2H, s), 7.70 (2H, d), 7.95 (2H, dt), 8.32 (2H, s) | m/z (ES+) (M + H)+ = (499.46; HPLC tR = 2.88 min. |

### Example 91

### Tert-butyl 4-(5-(4-(trifluoromethylsulfinyl)benzyloxy)pyrimidin-2-yl)piperazine-1-carboxylate

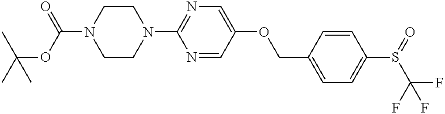

To a stirred solution of Tert-butyl 4-(5-(4-(trifluoromethylthio)benzyloxy)pyrimidin-2-yl)piperazine-1-carboxylate (Intermediate 57) (586 mg, 1.25 mmol) in dichloromethane (25.0 mL) at 0° C. was added a solution of 3-chloroperoxybenzoic acid (1228 mg, 4.98 mmol) in dichloromethane (25.0 mL). The mixture was stirred at ambient temperature for 16 hours, the dichloromethane evaporated in vacuo to a residue which was taken up in ethyl acetate (75 mL), washed with sodium metabisulphite solution, sodium hydrogen carbonate solution, brine, dried (MgSO₄) and evaporated in vacuo to a residue which was chromatographed on silica with 20% ethyl acetate in isohexane as eluant to give a product which was shown to be tert-butyl 4-(5-(4-(trifluoromethylsulfinyl)benzyloxy)pyrimidin-2-yl)piperazine-1-carboxylate (420 mg, 69%) plus recovered starting material (110 mg). ¹H NMR (CDCl₃) 1.4 (s, 9H), 3.4 (t, 4H), 3.65 (t, 4H), 5.05 (s, 2H), 7.6 (d, 2H), 7.75 (d, 2H) and 8.05 (s, 2H). m/z (ES+) (M+H)+=487; HPLC tR=2.86 min

### Example 92

### Tert-butyl 4-(5-(4-(2-morpholinoethylsulfonyl)benzyloxy)pyrimidin-2-yl)piperazine-1-carboxylate

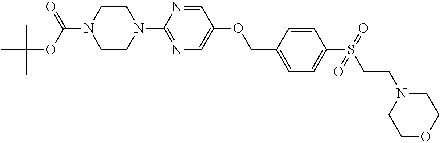

To a stirred solution of tert-butyl 4-(5-(4-(2-morpholinoethylsulfinyl)benzyloxy)pyrimidin-2-yl)piperazine-1-carboxylate (Intermediate 59) (0.335 g, 0.63 mmol) in methanol (0.790 mL) was added a solution of sodium tungstate dihydrate (4.16 mg, 0.01 mmol) in water (0.016 mL). The mixture was heated to 55° C. and treated with hydrogen peroxide (0.039 mL, 0.63 mmol) over 1 minute. When the addition was completed, the mixture was heated at 55° C. for 90 minutes, cooled to ambient temperature, the methanol evaporated in vacuo and the aqueous residue extracted with DCM (3×125 mL). The combined extracts were dried (MgSO₄) and evaporated. The crude product was purified by flash alumina chromatography, elution gradient 0 to 60% EtOAc in isohexane. Pure fractions were evaporated to dryness to afford tert-butyl 4-(5-(4-(2-morpholinoethylsulfonyl)benzyloxy)-pyrimidin-2-yl)piperazine-1-carboxylate (0.030 g, 9%) as a white solid. ¹H NMR (400.132 MHz, CDCl₃) 1.48 (9H, s), 2.36 (4H, t), 2.78 (2H, t), 3.30 (2H, t), 3.49 (4H, t), 3.54 (4H, t), 3.71 (4H, t), 5.12 (2H, s), 7.61 (2H, d), 7.95 (2H, d), 8.12 (2H, s). m/z (ES+) (M+H)+=548; HPLC tR=1.58 min.

### Example 93

### Tert-butyl 4-(5-(4-(methylsulfinyl)benzyloxy)pyrimidin-2-yl)piperazine-1-carboxylate

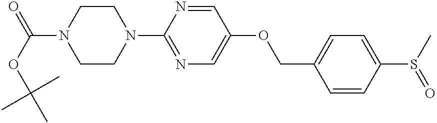

To a stirred solution of tert-butyl 4-(5-(4-(methylthio)benzyloxy)pyrimidin-2-yl)piperazine-1-carboxylate (Intermediate 60) (0.4 g, 0.96 mmol) in methanol (20 mL) was added a solution of sodium tungstate dihydrate (6.34 mg, 0.02 mmol) in water (0.2 mL). The mixture was stiffed at 20° C. and treated with hydrogen peroxide (0.059 mL, 0.96 mmol) over 1 minute. When the addition was completed, the mixture was stiffed at 20° C. for 60 minutes, treated with saturated sodium hydrogen carbonate solution (12 mL), the methanol evaporated in vacuo and the aqueous residue extracted with ethyl acetate (3×125 mL). The combined ethyl acetate extracts were dried (MgSO₄) and evaporated. The crude product was purified by flash silica chromatography, elution gradient 50 to 100% EtOAc in isohexane. Pure fractions were evaporated to dryness to afford tert-butyl 4-(5-(4-(methylsulfinyl)benzyloxy)pyrimidin-2-yl)piperazine-1-carboxylate (0.275 g, 66%) as a white solid. ¹H NMR (400.132 MHz, CDCl₃) 1.47 (9H, s), 2.74 (3H, s), 3.46-3.51 (4H, m), 3.68-3.74 (4H, m), 5.08 (2H, s), 7.57 (2H, d), 7.68 (2H, d), 8.12 (2H, s). m/z (ES+) (M+H)+=433; HPLC tR=3.03 min.

### Example 94

### Tert-butyl 4-(5-((2-isobutyramidopyrimidin-5-yl)methoxy)pyrimidin-2-yl)piperazine-1-carboxylate

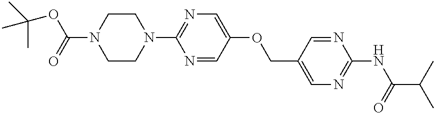

Isobutyryl chloride (0.022 mL, 0.21 mmol) was added to tert-butyl 4-(5-((2-aminopyrimidin-5-yl)methoxy)pyrimidin-2-yl)piperazine-1-carboxylate (Intermediate 62) (0.08 g, 0.10 mmol) and pyridine (0.033 mL, 0.41 mmol) in DCM (2 mL) at 20° C. The resulting solution was stirred at 20° C. for 2 hours. The reaction mixture was diluted with DCM (20 mL), and washed sequentially with water (15 mL) and saturated brine (20 mL). The organic layer was dried over Na₂SO₄, filtered and evaporated to afford crude product. The crude product was purified by flash silica chromatography, elution gradient 1 to 4% DCM in MeOH. Pure fractions were evaporated to dryness to afford tert-butyl 4-(5-((2-isobutyramidopyrimidin-5-yl)methoxy)pyrimidin-2-yl)piperazine-1-carboxylate (0.011 g, 23%) as a white solid. ¹H NMR (400.132 MHz, CDCl₃) 1.28 (6H, d), 1.48 (9H, s), 2.94 (1H, septet), 3.46-3.50 (4H, m), 3.68-3.75 (4H, m), 4.97 (2H, s), 8.04 (1H, s), 8.12 (2H, s), 8.64 (2H, s). m/z (ES+) (M+H)+=458; HPLC tR=3.03 min.

### Example 95

### Tert-butyl 4-(5-((3-methylpyridin-4-yl)methoxy)pyrimidin-2-yl)piperazine-1-carboxylate

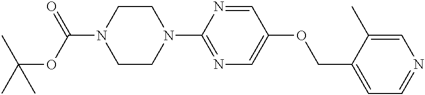

Borane-THF complex, 1M in THF (5.83 mL, 5.83 mmol) was added to 3-methylisonicotinic acid (400 mg, 2.92 mmol) in THF (5.0 mL) at 5° C. over a period of 5 minutes under nitrogen. The resulting solution was stiffed and allowed to warm to ambient temperature over 18 hours. Methanol (20 mL) was added cautiously followed by 4M HCl in dioxane (10 drops) and the mixture stirred for 30 minutes. It was concentrated in vacuo and adsorbed onto silica. The crude 3-methyl-4-pyridylmethanol was purified by flash silica chromatography, elution gradient 0 to 100% EtOAc in isohexane. Pure fractions were evaporated to dryness to afford 156 mg of a white solid. To this was added diisopropyl azodicarboxylate (0.240 mL, 1.22 mmol), tert-butyl 4-(5-hydroxypyrimidin-2-yl)piperazine-1-carboxylate (Intermediate 1) (341 mg, 1.22 mmol) and triphenylphosphine (0.271 mL, 1.24 mmol) in THF (10.0 mL) at 0° C. over a period of 2 minutes under nitrogen. The resulting solution was stirred and warmed to ambient temperature over 18 hours. It was diluted with DCM (100 mL) and washed with brine (30 mL), dried (sodium sulphate), concentrated in vacuo and adsorbed onto silica. The crude product was purified by flash silica chromatography, elution gradient 0 to 100% EtOAc in isohexane. Pure fractions were evaporated to dryness to afford a white solid (285 mg). This was suspended in DMSO-MeCN-Water 7:2:1 (7.5 mL, 0.71 mmol) and stirred at 100° C. for 18 hours. It was cooled to room temperature and the resulting white precipitate collected by filtration and dried under vacuum. This gave tert-butyl 4-(5-((3-methylpyridin-4-yl)methoxy)pyrimidin-2-yl)piperazine-1-carboxylate (198 mg, 72%) as a white solid. ¹H NMR (400 MHz, DMSO) 1.4 (s, 9H), 2.25 (s, 3H), 3.4 (t, 4H), 3.6 (t, 4H), 5.15 (s, 2H), 7.4 (d, 1H), 8.3 (s, 2H), 8.4 (m, 2H). m/z (ES+) (M+H)+=386.25; HPLC tR=2.66 min.

### Example 96

### (R)-4-((2-(4-(5-(difluoromethyl)-1,2,4-oxadiazol-3-yl)-2-methylpiperazin-1-yl)pyrimidin-5-yloxy)methyl)nicotinonitrile

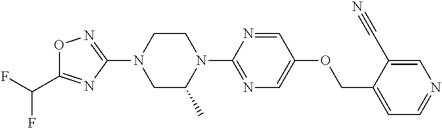

Cesium carbonate (1.377 g, 4.23 mmol) was added to 4-(chloromethyl)nicotinonitrile (1.280 g, 3.52 mmol) and (R)-2-(4-(5-(difluoromethyl)-1,2,4-oxadiazol-3-yl)-2-methylpiperazin-1-yl)pyrimidin-5-ol (1.1 g, 3.52 mmol, INT 87) in DMF (20 mL). The resulting mixture was stirred at 20° C. for 20 hours. The reaction mixture was quenched with water (15 mL), extracted with EtOAc (2×20 ml), the organic layer was dried over MgSO₄, filtered and evaporated to afford a beige solid. Purified by preparative HPLC (Phenomenex Gemini C18 110A (axia) column, 5μ silica, 30 mm diameter, 100 mm length), using decreasingly polar mixtures of water (containing 0.5% NH3) and MeCN as eluents. Fractions containing the desired compound were neutralised with 1M HCl, the acetonitrile evaporated and the residue extracted into DCM, dried and evaporated to dryness to afford the product as a yellow gum which was re-purified by flash alumina chromatography, elution gradient 10 to 50% EtOAc in isohexane. Pure fractions were evaporated to dryness to afford (R)-4-((2-(4-(5-(difluoromethyl)-1,2,4-oxadiazol-3-yl)-2-methylpiperazin-1-yl)pyrimidin-5-yloxy)methyl)nicotinonitrile (0.510 g, 34%) as a white solid. ¹H NMR (400 MHz, CDCl₃) 1.25 (3H, d), 3.10-3.17 (1H, m), 3.27-3.37 (2H, m), 3.86-3.91 (1H, m), 4.01-4.05 (1H, m), 4.49-4.54 (1H, m), 4.91-4.99 (1H, m), 5.21 (2H, s), 6.65 (1H, t), 7.66 (1H, d), 8.20 (2H, s), 8.85 (1H, d), 8.91 (1H, s). m/z (ES+) (M+H)+=429.38; HPLC tR=2.46 min.

### Example 97

### (R)-4-((2-(4-(5-cyclopropyl-1,2,4-oxadiazol-3-yl)-2-methylpiperazin-1-yl)pyrimidin-5-yloxy)methyl)nicotinonitrile

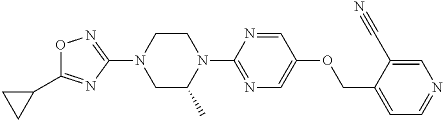

(R)-3-(4-(5-((3-bromopyridin-4-yl)methoxy)pyrimidin-2-yl)-3-methylpiperazin-1-yl)-5-cyclopropyl-1,2,4-oxadiazole (350 mg, 0.74 mmol), zinc cyanide (87 mg, 0.74 mmol), 9,9-dimethyl-4,5-bis(diphenylphosphino)xanthene (34.3 mg, 0.06 mmol) and tris(dibenzylideneacetone)dipalladium(0) (27 mg, 0.03 mmol) were suspended in DMF (5.0 mL), degassed with nitrogen for 15 minutes and sealed into a microwave tube. The reaction was heated at 130° C. for 1 hour in the microwave reactor and cooled to RT. The rection mixture was diluted with ethyl acetate (150 mL) and washed with water (30 mL) and brine (30 mL), dried (magnesium sulphate), concentrated in vacuo and adsorbed onto silica. The crude product was purified by flash silica chromatography, elution gradient 0 to 100% EtOAc in isohexane. Pure fractions were evaporated to dryness to afford (R)-4-((2-(4-(5-cyclopropyl-1,2,4-oxadiazol-3-yl)-2-methylpiperazin-1-yl)pyrimidin-5-yloxy)methyl)nicotinonitrile (168 mg, 54%) as a white solid. ¹H NMR (400.13 MHz, DMSO-d6) 1.15 (7H, m), 2.2 (1H, m), 3.0 (1H, m), 3.2 (2H, m), 3.75 (1H, d), 3.9 (1H, d), 4.4 (1H, d), 4.85 (1H, m), 5.4 (2H, s), 7.8 (1H, d), 8.4 (2H, s), 8.9 (1H, d), 9.1 (1H, s). m/z (ES+) (M+H)+=419.48; HPLC tR=2.43 min.

### Example 98

### (R)-4-((2-(4-(3-isopropyl-1,2,4-oxadiazol-5-yl)-2-methylpiperazin-1-yl)pyrimidin-5-yloxy)methyl)nicotinonitrile

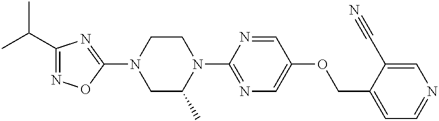

Potassium carbonate (0.601 g, 4.35 mmol) was added to (R)-2-(4-(3-isopropyl-1,2,4-oxadiazol-5-yl)-2-methylpiperazin-1-yl)pyrimidin-5-ol (0.441 g, 1.45 mmol, INT 69) and 4-(chloromethyl)nicotinonitrile (0.608 g, 1.59 mmol) in acetonitrile (20 mL) under nitrogen. The resulting solution was stirred at ambient temperature for 18 hours. The reaction mixture was concentrated and diluted with EtOAc (50 mL), and washed sequentially with water (50 mL) and saturated brine (50 mL). The organic layer was dried over MgSO₄, filtered and evaporated to afford crude product. The crude product was purified by flash silica chromatography, elution gradient 0 to 30% EtOAc in DCM. Pure fractions were evaporated to dryness to afford (R)-4-((2-(4-(3-isopropyl-1,2,4-oxadiazol-5-yl)-2-methylpiperazin-1-yl)pyrimidin-5-yloxy)methyl)nicotinonitrile (0.037 g, 6%) as a pale yellow solid. ¹H NMR (400 MHz, CDCl₃) 1.23 (3H, d), 1.30 (6H, d), 2.91 (1H, dt), 3.27 (2H, tdd), 3.41 (1H, dd), 3.96 (1H, dd), 4.04-4.21 (1H, m), 4.43-4.62 (1H, m), 4.82-5.03 (1H, m), 5.22 (2H, s), 7.58-7.76 (1H, m), 8.20 (2H, s), 8.85 (1H, d), 8.91 (1H, s). m/z (ES+) (M+H)+=421.4; HPLC tR=2.33 min.

### Example 99

### (R)-4-((2-(2-methyl-4-(3-(trifluoromethyl)-1,2,4-oxadiazol-5-yl)piperazin-1-yl)pyrimidin-5-yloxy)methyl)nicotinonitrile

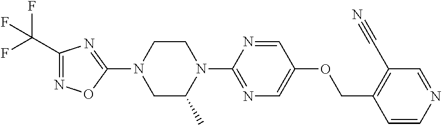

Potassium carbonate (31.4 mg, 0.23 mmol) was added to (R)-2-(2-methyl-4-(3-(trifluoromethyl)-1,2,4-oxadiazol-5-yl)piperazin-1-yl)pyrimidin-5-ol (25 mg, 0.08 mmol) and 4-(chloromethyl)nicotinonitrile (23.10 mg, 0.15 mmol) in acetonitrile (1 mL) at 20° C. The resulting suspension was stirred at 60° C. for 90 minutes. The reaction mixture was diluted with DCM (10 mL), and washed with water (5 mL). The organic layer was purified by flash silica chromatography, elution gradient 0 to 60% EtOAc in isohexane. Pure fractions were evaporated to dryness to afford (R)-4-((2-(2-methyl-4-(3-(trifluoro-methyl)-1,2,4-oxadiazol-5-yl)piperazin-1-yl)pyrimidin-5-yloxy)methyl)nicotinonitrile (17 mg, 50%) as a colourless gum. ¹H NMR (400.13 MHz, CDCl₃) 1.17 (3H, d), 3.21-3.31 (2H, m), 3.44 (1H, dd), 3.94 (1H, dt), 4.12 (1H, d), 4.45-4.55 (1H, m), 4.90-4.98 (1H, m), 5.16 (2H, s), 7.59 (1H, dd), 8.14 (2H, s), 8.79 (1H, d), 8.85 (1H, s). m/z (ES+) (M+H)+=447.42; HPLC tR=2.63 min.

### Example 100

### 4-((2-((R)-4-(5-((S)-1-methoxyethyl)-1,2,4-oxadiazol-3-yl)-2-methylpiperazin-1-yl)pyrimidin-5-yloxy)methyl)nicotinonitrile

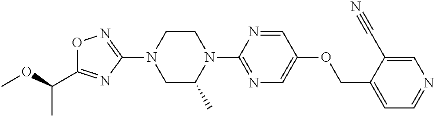

3-((R)-4-(5-((3-bromopyridin-4-yl)methoxy)pyrimidin-2-yl)-3-methylpiperazin-1-yl)-5-((S)-1-methoxyethyl)-1,2,4-oxadiazole (0.180 g, 0.37 mmol), was placed in a round bottomed flask and to it was added dicyanozinc (0.034 g, 0.29 mmol), tris(dibenzylideneacetone)dipalladium(0) (0.013 g, 0.01 mmol), (9,9-dimethyl-9H-xanthene-4,5-diyl)bis(diphenylphosphine) (0.017 g, 0.03 mmol) and zinc (2.53 μL, 0.04 mmol) and the solids degassed then DMA (2 mL) and water (0.02 mL) and the mixture degassed again (N₂/vacuum). The reaction was heated to 130° C. for 90 minutes then diluted with DCM (50 mL) and washed with 2M K₂CO₃ aq (20 ml). The organic layer was dried over MgSO₄, filtered and evaporated to afford crude product. The crude product was purified by preparative HPLC (Waters XBridge Prep C18 OBD column, 5μ silica, 50 mm diameter, 150 mm length), using decreasingly polar mixtures of water (containing 0.5% NH₃) and MeCN as eluents. Fractions containing the desired compound were combined and the pH adjusted to ˜7 with 1M HCl aq. The bulk of the organic solvent was removed under reduced pressure to give a white suspension. The suspension was extracted with DCM (2×25 ml) and the combined organics dried over Na₂SO₄, filtered and evaporated to afford 4-((2-((R)-4-(5-((S)-1-methoxyethyl)-1,2,4-oxadiazol-3-yl)-2-methylpiperazin-1-yl)pyrimidin-5-yloxy)methyl)nicotinonitrile (0.125 g, 78%), as a very pale brown solid. ¹H NMR (400 MHz, CDCl₃, 30° C.) 1.25 (3H, d), 1.58 (3H, d), 3.08 (1H, td), 3.21-3.36 (2H, m), 3.43 (3H, s), 3.89 (1H, dt), 4.00-4.06 (1H, m), 4.45-4.54 (2H, m), 4.88-4.96 (1H, m), 5.22 (2H, s), 7.66 (1H, dd), 8.20 (2H, s), 8.85 (1H, d), 8.91 (1H, d). m/z (ES+) (M+H)+=437.

The following Example was prepared in a similar manner to Example 100, using the Intermediate stated:

| Structure and INT | Ex | Name | 1H NMR δ | MS |
|---|---|---|---|---|
| 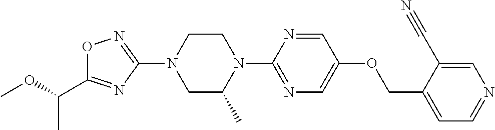 INT 72 | 101 | 4-((2-((R)-4- (5-((R)-1- methoxyethyl)- 1,2,4-oxadiazol- 3-yl)-2- mcthylpiperazin- 1-yl)pyrimidin- 5-yloxy)methyl) nicotinonitrile | 1H NMR (400 MHz, CDCl3, 30° C.) d 1.25 (3H, d), 1.57 (3H, d), 3.08 (1H, td), 3.20- 3.39 (2H, m), 3.43 (3H, s), 3.88 (1H, dt), 3.96-4.08 (1H, m), 4.42-4.59 (2H, m), 4.91 (1H, ddd), 5.22 (2H, s), 7.62- 7.68 (1H, m), 8.20 (2H, s), 8.85 (1H, d), 8.91 (1H, s). | m/z (ES+) (M + H)+ = 437 |

### Example 102

### (R)-4-((2-(2-methyl-4-(5-(trifluoromethyl)-1,2,4-oxadiazol-3-yl)piperazin-1-yl)pyrimidin-5-yloxy)methyl)nicotinonitrile

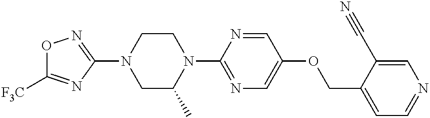

Cesium carbonate (5.49 g, 16.86 mmol) was added to 4-(chloromethyl)nicotinonitrile (2.144 g, 14.05 mmol) and (R)-2-(2-methyl-4-(5-(trifluoromethyl)-1,2,4-oxadiazol-3-yl)piperazin-1-yl)pyrimidin-5-ol (4.64 g, 14.05 mmol, INT 73) in DMF (60 mL). The resulting mixture was stiffed at 20° C. for 70 hours. The reaction mixture was quenched with water (15 mL), extracted with EtOAc (2×20 ml). The organic layer was dried over MgSO₄, filtered and evaporated to afford a beige solid that was purified by flash silica chromatography, elution gradient 10 to 30% EtOAc in DCM. The oil obtained was triturated with isohexane/Et₂O to give a solid which was collected by filtration and dried under vacuum to give (R)-4-((2-(2-methyl-4-(5-(trifluoromethyl)-1,2,4-oxadiazol-3-yl)piperazin-1-yl)pyrimidin-5-yloxy)methyl)nicotinonitrile (3.43 g, 55%) as a white solid. ¹H NMR (400 MHz, CDCl₃) 1.25 (3H, d), 3.09-3.20 (1H, m), 3.27-3.40 (2H, m), 3.86-3.91 (1H, m), 4.00-4.06 (1H, m), 4.48-4.56 (1H, m), 4.91-4.99 (1H, m), 5.22 (2H, s), 7.66 (1H, d), 8.20 (2H, s), 8.85 (1H, d), 8.91 (1H, s). m/z (ES+) (M+H)+=447.

### Example 103

### (R)—((R)-1,1,1-trifluoropropan-2-yl) 4-(5-((3-cyanopyridin-4-yl)methoxy)pyrimidin-2-yl)-3-methylpiperazine-1-carboxylate

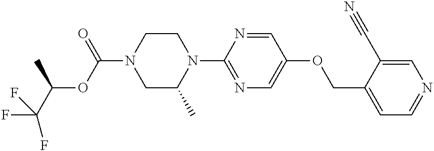

Triethylamine (0.582 mL, 4.17 mmol) was added to 4-((2-(2-methylpiperazin-1-yl)pyrimidin-5-yloxy)methyl)nicotinonitrile dihydrochloride (0.297 g, 0.80 mmol, INT 65), (R)-phenyl 1,1,1-trifluoropropan-2-yl carbonate (0.367 g, 1.57 mmol) and (R)-1,1,1-trifluoro-2-propanol (0.189 mL, 2.09 mmol) in chloroform (5 mL) under nitrogen. The solution was stirred at 80° C. for 18 hours during which time the solvent had evaporated slightly to leave a dark solution. The reaction mixture was diluted with DCM (50 mL), and washed with 2M K₂CO₃ aq. (20 ml). The organic layer was dried over Na₂SO₄ and evaporated to afford crude product. The crude product was purified by preparative HPLC (Waters XBridge Prep C18 OBD column, 5μ silica, 50 mm diameter, 150 mm length), using decreasingly polar mixtures of water (containing 0.5% NH₃) and MeCN as eluents. The product fractions were combined and the pH adjusted to ˜7 with 2M HCl aq and 1M NaHCO₃ aq. The bulk of the organic solvent was removed under reduced pressure to give a white suspension. The suspension was extracted with DCM (2×50 mL) and the combined organics dried over Na₂SO₄, filtered and evaporated then triturated with Et₂O to give a solid which was dried under vacuum to give (R)—((R)-1,1,1-trifluoropropan-2-yl) 4-(5-((3-cyanopyridin-4-yl)methoxy)pyrimidin-2-yl)-3-methylpiperazine-1-carboxylate (0.214 g, 43%) as a white solid. ¹H NMR (400 MHz, DMSO, 30° C.) 9.12 (s, 1H), 8.94 (d, 1H), 8.41 (s, 2H), 7.80 (d, 1H), 5.42 (d, 1H), 5.40 (s, 2H), 4.81 (s, 1H), 4.37 (d, 1H), 4.15-3.95 (m, 1H), 3.89 (d, 1H), 3.27 (s, 1H), 3.18-2.97 (m, 2H), 1.43 (d, 3H), 1.12 (d, 3H). m/z (ES+) (M+H)+=451

The following Example was prepared in a similar manner to Example 103, using the Intermediate stated and (S)-phenyl 1,1,1-trifluoropropan-2-yl carbonate and (S)-1,1,1-trifluoro-2-propanol:

| Structure and INT | Ex | Name | 1H NMR δ | MS |
|---|---|---|---|---|
| 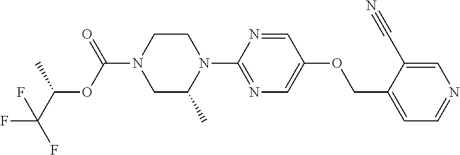 INT 65 | 104 | (R)-((S)-1,1,1- trifluoropropan- 2-yl)4-(5-((3- cyanopyridin-4- yl)methoxy) pyrimidin-2- yl)-3- mcthylpipcrazine- 1-carboxylate | 1H NMR (400.13 MHz)(DMSO-d6) 1.05 (3H, d), 1.35 (3H, d), 2.95-3.3 (3H, m), 3.8-4.05 (2H, m), 4.3 (1H, m), 4.75 (1H, m), 5.3 (3H, m), 7.7 (1H, d), 8.3 (2H, s), 8.85 (1H, d), 9.05 (1H, s) | m/z (ES+) (M + H)+ = 451 |

The (R) and (S)-phenyl 1,1,1-trifluoropropan-2-yl carbonates were made according to the same procedure as INT 39 using (R) and (S)-1,1,1-trifluoro-2-propanol, respectively.

### Example 105

### (R)-2,2,2-trifluoroethyl 4-(5-((3-cyanopyridin-4-yl)methoxy)pyrimidin-2-yl)-3-methylpiperazine-1-carboxylate

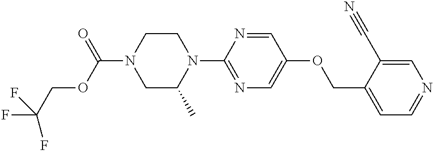

Triethylamine (0.633 mL, 4.54 mmol) was added to (R)-4-((2-(2-methylpiperazin-1-yl)pyrimidin-5-yloxy)methyl)nicotinonitrile hydrochloride (394 mg, 1.14 mmol, INT 65), phenyl 2,2,2-trifluoroethyl carbonate (487 mg, 1.70 mmol) in 2,2,2-trifluoroethanol (4.97 mL, 68.16 mmol) and chloroform (3 mL) under nitrogen. The reaction was stirred at 90° C. for 18 hours during which time the solvent had evaporated slightly to leave a brown clear solution. The reaction mixture was diluted with DCM (50 mL), and washed with 2M K₂CO₃ aq. (20 ml). The organic layer was dried over Na₂SO₄, filtered and evaporated to afford crude product. The crude product was purified by flash silica chromatography, elution gradient 0 to 100% EtOAc in isohexane (quickly to 70% then more slowly to 100%). Pure fractions were evaporated to dryness to give a pale yellow solid that was triturated with Et₂O to give a solid which was collected by filtration and washed with isohexane and dried under vacuum to give (R)-2,2,2-trifluoroethyl 4-(5-((3-cyanopyridin-4-yl)methoxy)pyrimidin-2-yl)-3-methylpiperazine-1-carboxylate (193 mg, 38.9%) as a white solid. ¹H NMR (400 MHz, DMSO, 100° C.) 1.10 (3H, d), 3.03-3.35 (3H, m), 3.83 (1H, d), 3.97 (1H, d), 4.33 (1H, d), 4.53-4.96 (3H, m), 5.31 (2H, s), 7.72 (1H, d), 8.31 (2H, s), 8.85 (1H, d), 8.99 (1H, s). m/z (ES+) (M+H)+=437

The following Example was prepared in a similar manner to Example 105, using the Intermediate stated

| Structure and INT | Ex | Name | 1H NMR δ | MS |
|---|---|---|---|---|
| 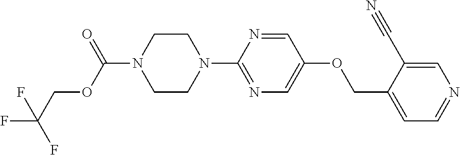 INT 64 | 106 | 2,2,2-trifluorocthyl 4-(5-((3- cyanopyridin-4- yl)mcthoxy) pyrimidin-2- yl)pipcrazinc-1- carboxylate | ¹H NMR (400.13 MHz, CDC1₃) 3.5 (4H, dd), 3.7 (4H, br), 4.4- 4.5 (2H, q), 5.15 (2H, s), 7.6 (1H, d), 8.1 (2H, s), 8.75 (1H, d), 8.8 (1H, s) | m/z (ES−) (M − H)− = 421 |

### Example 107

### (R)-4-((2-(2-methyl-4-(5-(3-methyloxetan-3-yl)-1,2,4-oxadiazol-3-yl)piperazin-1-yl)pyrimidin-5-yloxy)methyl)nicotinonitrile

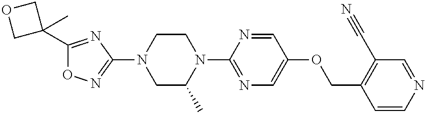

Cesium carbonate (0.217 mL, 2.71 mmol) was added to (R)-2-(2-methyl-4-(5-(3-methyloxetan-3-yl)-1,2,4-oxadiazol-3-yl)piperazin-1-yl)pyrimidin-5-ol (300 mg, 0.90 mmol, INT 74) and 4-(chloromethyl)nicotinonitrile (1290 mg, 8.45 mmol) in acetonitrile (10.0 mL). The resulting suspension was stirred at 20° C. for 3 days then it was concentrated in vacuo and adsorbed onto silica. The crude product was purified by flash silica chromatography, elution gradient 0 to 100% EtOAc in isohexane. Pure fractions were evaporated to dryness to afford (R)-4-((2-(2-methyl-4-(5-(3-methyloxetan-3-yl)-1,2,4-oxadiazol-3-yl)piperazin-1-yl)pyrimidin-5-yloxy)methyl)nicotinonitrile (123 mg, 30%) as a white solid. ¹H NMR (400.13 MHz) (DMSO-d6) 1.15 (3H, d), 1.7 (3H, s), 3.0 (1H, m), 3.2 (2H, m), 3.7 (1H, d), 3.9 (1H, d), 4.4 (1H, d), 4.5 (2H, d), 4.85 (3H, m), 5.35 (2H, s), 7.7 (1H, d), 8.35 (2H, s), 8.85 (1H, d), 9.05 (1H, s). m/z (ES+) (M+H)+=449.

### Example 108

### (R)-3-(trifluoromethyl)oxetan-3-yl 4-(5-(2-fluoro-4-(methylsulfonylmethyl)benzyloxy)-pyrimidin-2-yl)-3-methylpiperazine-1-carboxylate

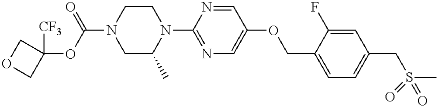

HCl (4M in dioxane) (3 mL) was added to (R)-tert-butyl 4-(5-(2-fluoro-4-(methylsulfonylmethyl)benzyloxy)pyrimidin-2-yl)-3-methylpiperazine-1-carboxylate (0.220 g, 0.44 mmol, INT 75) in DCM (7 mL). The resulting solution was stirred at 20° C. for 18 hours giving a pale yellow gum. It was concentrated in vacuo, azeotroped once with toluene and dried under vacuum to give a white solid. To this was added triethylamine (0.368 mL, 2.64 mmol) and perfluorophenyl 3-(trifluoromethyl)oxetan-3-yl carbonate (0.163 g, 0.44 mmol) in chloroform (5 mL) at 20° C. The reaction was stirred at 20° C. for 18 hours. The reaction mixture was diluted with DCM (50 mL), and washed with 2M K₂CO₃ (20 mL). The organic layer was dried over MgSO₄, filtered and evaporated to afford crude material. The crude product was purified by flash silica chromatography, elution gradient 0 to 40% EtOAc in DCM. Pure fractions were evaporated to dryness to afford (0.232 g, 94%) as a white solid. ¹H NMR (400 MHz, DMSO, 100° C.) 1.11 (3H, d), 2.90 (3H, s), 3.04-3.30 (3H, m), 3.77-3.85 (1H, m), 3.91-3.98 (1H, m), 4.28-4.35 (1H, m), 4.49 (2H, s), 4.73-4.82 (3H, m), 4.94-5.00 (2H, m), 5.15 (2H, s), 7.27-7.33 (2H, m), 7.56 (1H, t), 8.26 (2H, s). m/z (ES+) (M+H)+=563.

The following Examples were prepared in a similar manner to Example 108, using the Intermediates stated

| Structure and INT | Ex | Name | 1H NMR δ | MS |
|---|---|---|---|---|
| 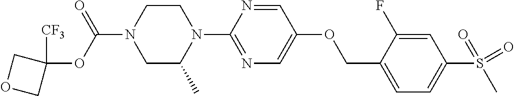 INT 75 | 109 | (R)-3- (trifluoromcthyl) oxctan-3-yl 4- (5-(2-fluoro-4- (methylsulfonyl) bcnzyloxy) pyrimidin-2-yl)- 3-mcthyl- piperazine- 1-carboxylate | 1H NMR (400 MHz, CDCl3, 30° C.) 1.11- 1.25 (3H, m), 3.07 (3H, d), 3.18 (2H, d), 3.29 (1H, s), 4.01 (2H, ddd), 4.41 (1H, t), 4.84 (1H, d), 4.85-5.15(4H, m), 5.16 (2H, s), 7.73 (3H, ddd), 8.16 (2H, s). | m/z (ES+) (M + H)+ = 549 |
| 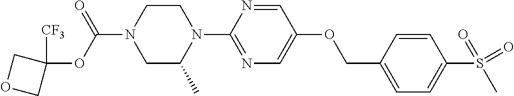 INT 36 | 110 | (R)-3- (trifluoromethyl) oxetan-3-yl 3- mcthyl-4-(5-(4- (mcthylsulfonyl) benzyloxy) pyrimidin-2- yl)piperazine-1- carboxylate | 1H NMR (400 MHz, CDCl3) 1.16 (3H, d), 3.06 (3H, s), 2.92-3.31 (3H, m), 3.85-3.97 (1H, m), 4.09 (1H, dd), 4.39 (1H, t),4.86 (3H, dd), 4.97 (1H, t), 5.04 (1H, s), 5.12 (2H, s), 7.62 (2H, d), 7.96-8.01 (2H, m), 8.13 (2H, s). | m/z (ES+) (M + H)+ = 531 |
| 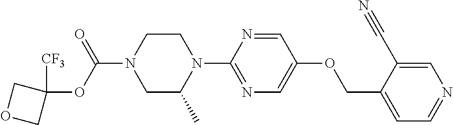 INT 65 | 111 | (R)-3- (trifluoromcthyl) oxetan-3-yl 4- (5-((3- cyanopyridin-4- yl)mcthoxy) pyrimidin-2-yl)- 3-mcthylpipcrazine- 1-carboxylate | 1H NMR (400 MHz, CDCl3) 1.18 (3H, d), 2.94-3.31 (3H, m), 3.85-4.19 (2H, m), 4.37- 4.49 (1H, m), 4.81-4.91 (3H, m), 4.94-4.99 (1H, m), 5.03- 5.07 (1H, m), 5.22 (2H, s), 7.66 (1H, d), 8.19 (2H, s), 8.85 (1H,d), 8.91 (1H, s) | m/z (ES+) (M + H)+ = 479 |

### Example 112

### (R)-4-((2-(4-(5-isopropyl-1,2,4-oxadiazol-3-yl)-2-methylpiperazin-1-yl)pyrimidin-5-yloxy)methyl)nicotinonitrile

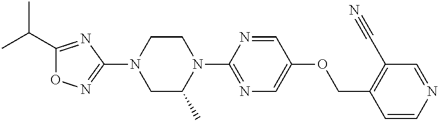

To a stirred solution of (R)-5-isopropyl-3-(3-methylpiperazin-1-yl)-1,2,4-oxadiazole (106 mg, 0.50 mmol) (Intermediate 82) and 4-((2-chloropyrimidin-5-yloxy)methyl)nicotinonitrile (124 mg, 0.50 mmol) (Intermedaite 84) in 2-propanol (1.264 mL) in a microwave vial was added N,N-diisopropylethylamine (0.250 mL, 1.51 mmol) and the mixture heated in a Biotage Initiator Microwave at 140° C. for 40 hours. The mixture was cooled to ambient temperature, poured onto ethyl acetate (30 mL), washed with water (5 mL), brine (5 mL), dried (MgSO₄) and evaporated to a residue which was chromatographed on silica with 50% ethyl acetate in isohexane then by basic reverse-phase chromatography to give (R)-4-((2-(4-(5-isopropyl-1,2,4-oxadiazol-3-yl)-2-methylpiperazin-1-yl)pyrimidin-5-yloxy)methyl)nicotinonitrile (95 mg, 45%).

Intermediates

Int 1

### Tert-butyl 4-(5-hydroxypyrimidin-2-yl)piperazine-1-carboxylate

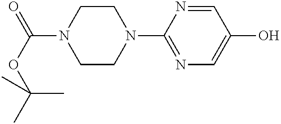

To a mixture of tert-butyl 4-(5-bromopyrimidin-2-yl)piperazine-1-carboxylate (17.16 g, 50 mmol), bis(pinacolato)diboron (14.09 g, 55.50 mmol), potassium acetate (14.92 g, 152.00 mmol) and palladium(II) acetate (0.370 g, 1.65 mmol) under an atmosphere of nitrogen was added DMF (185 mL). The stirred mixture was heated at 85° C. for 16 hours, cooled to ambient temperature, poured onto water (2775 mL), extracted with ethyl acetate (2×750 mL), the combined ethyl acetate extracts washed with brine, dried (MgSO₄) and evaporated in vacuo to a residue which was taken up in a mixture of tetrahydrofuran (370 mL) and water (370 mL) and treated with sodium perborate tetrahydrate (19.62 g, 127.50 mmol). The mixture was stirred at ambient temperature for 16 hours. The tetrahydrofuran evaporated in vacuo, then the aqueous residue was treated with saturated ammonium chloride solution (500 mL) and extracted with ethyl acetate (3×350 mL). The combined ethyl acetate extracts were washed with brine, dried (MgSO₄) and evaporated in vacuo to a residue which was chromatographed on silica with 50% ethyl acetate in isohexane as eluant to give tert-butyl 4-(5-hydroxypyrimidin-2-yl)piperazine-1-carboxylate (3.13 g, 23%). ¹H NMR (500 MHz CDCl₃) 1.47 (s, 9H), 3.41-3.50 (m, 4H), 3.60-3.70 (m, 4H), 6.64 (s, 1H), 8.05 (s, 2H). m/z (ES−) (M−H)−=279; HPLC tR=1.93 min.

The following pyrimidin-5-ol intermediates were prepared in a similar manner to Int 1, using an appropriate bromo intermediate

| INTERMEDIATE | Name | 1H NMR δ | MS |
|---|---|---|---|
| 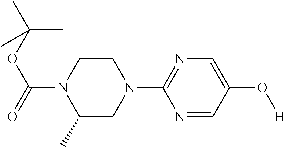 INT 2 | (S)-tert-butyl 4-(5- hydroxypyrimidin- 2-yl)-2- methylpipcrazine- 1-carboxylate | | m/z (ESI−) (M − H)− = 293; HPLC tR = 1.60 min. |
| 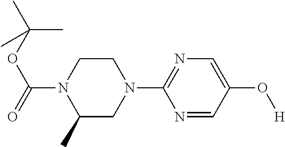 INT 3 | (R)-tert-butyl 4-(5- hydroxypyrimidin- 2-yl)-2- mcthylpipcrazine- 1-carboxylate | | m/z (ESI−) (M − H)− = 293; HPLC tR = 1.60 min. |
| 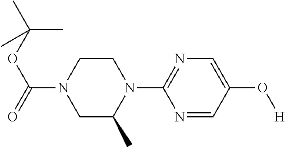 INT 4 | (S)-tert-butyl 4-(5- hydroxypyrimidin- 2-yl)-3- mcthylpipcrazine- 1-carboxylate | 1H NMR (400.13 MHz, DMSO-d6) 1.05-1.07 (3H, m), 1.42 (9H, s), 2.75-3.15 (3H, m), 3.74-4.02 (3H, m), 4.32-4.40 (1H, m), 4.74-4.83 (1H, m), 6.63 (1H, t), 8.37 (2H, d) | m/z (ES−) (M − H)− = 279; HPLC tR = 1.93 min. |
| 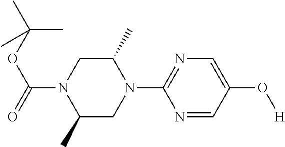 INT 5 | (2R,5S)-tert-butyl 4-(5- hydroxypyrimidin- 2-yl)-2,5- dimcthylpiperazine- 1-carboxylate | 1H NMR (400.132 MHz, CDCl3) 1.10-1.18 (6H, m), 1.48 (9H, s), 3.22- 3.36 (2H, m), 3.68-3.82 (1H, m), 4.10-4.32 (2H, m), 4.66-4.80 (1H, m), 5.92 (1H, bs), 8.07 (2H, s) | m/z (ES+) (M + H)+ = 309; HPLC tR = 2.87 min. |
| 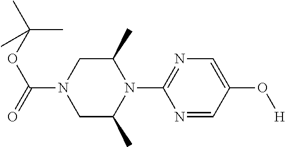 INT 6 | (3R,5S)-tert-butyl 4-(5- hydroxypyrimidin- 2-yl)-3,5- dimcthylpipcrazine- 1-carboxylate | 1H NMR (400.13 MHz, CDCl3) 1.24 (9H, s), 1.51 (6H, d), 3.05 (2H, s), 4.03 (2H, bs), 4.63 (1H, s), 4.80 (1H, s), 8.11 (2H, s) | m/z (ESI+) (M + H)+ = 309; HPLC tR = 1.76 min. |
| 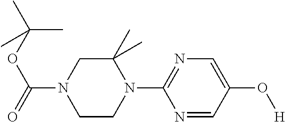 INT 7 | tert-butyl 4-(5- hydroxypyrimidin- 2-yl)-3,3- dimcthylpipcrazine- 1-carboxylate | 1H NMR (400.13 MHz, CDCl3) 1.25 (6H, s), 1.48 (9H, s), 2.05 (1H, s), 3.41- 3.62 (4H, m), 3.89 (2H, t), 8.07 (2H, s) | m/z (ESI+) (M + H)+ = 309; HPLC tR = 1.59 min. |
| 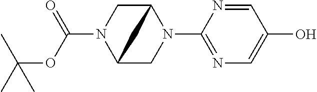 INT 8 | (1R,4R)-tert-butyl 5-(5- hydroxypyrimidin- 2-yl)-2,5- diazabicyclo[2.2.1] hcptanc-2- carboxylate | 1H NMR (400 MHz, DMSO) 1.45 (m, 9H), 1.95 (m, 2H), 3.2 (d, 1H), 3.4 (m, 2H),3.5 (m, 1H), 4.4 (m, 1H),4.75 (s, 1H), 8.05 (s, 2H), 9.2 (s, 1H). | m/z (ESI+) (M + H)+ = 293.18; HPLC tR = 1.36 min. |
| 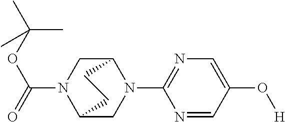 INT 9 | (1S,4S)-tcrt-butyl 5-(5- hydroxypyrimidin- 2-yl)-2,5- diazabicyclo[2.2.2] octane-2- carboxylate | 1H NMR(CDCl3) 1.4 (d, 9H), 1.75 (m, 2H), 1.95 (m, 2H), 2.9 (d, 1H), 3.5 (m, 2H), 3.7 (d, 1H), 4.25 (d, 1H),4.75 (d, 1H), 6.25 (br, 1H) and 7.95 (s, 2H). | m/z (ES+) (M + H)+ = 307; HPLC tR = 1.56 min. |

Int 10

### (R)-Tert-butyl 4-(5-hydroxypyrimidin-2-yl)-3-methylpiperazine-1-carboxylate

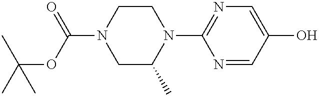

A 1.6M solution of n-Butyllithium (28.7 mL, 45.99 mmol) in hexane was added dropwise to a stiffed solution of (R)-tert-butyl 4-(5-bromopyrimidin-2-yl)-3-methylpiperazine-1-carboxylate (13.69 g, 38.32 mmol) (Intermediate 11) and triisopropyl borate (10.61 mL, 45.99 mmol) in THF (100 mL) cooled to −55° C., over a period of 10 minutes under nitrogen. The internal temperature was not allowed to rise above −44° C. The resulting solution was stirred at −45° C. for 40 minutes. The temperature was allowed to warm to −20° C. whereupon glacial acetic acid (4.1 mL) was added. The reaction was rinsed in to a single necked round bottomed flask (with THF/MeOH) then evaporated to a yellow solid. This was azeotroped with MeOH (200 mL) and the resultant pale yellow solid dissolved in MeOH (40 mL). Water (140 mL) was added but the product appeared to ‘stick’ to the sides of the flask. Hydrogen peroxide (3.39 mL, 38.32 mmol) was added dropwise with manual ‘swirling’ but upon complete addition none of the gum had appeared to dissolve. MeOH (˜20-30 mL) was rinsed down the side of the flask and gradually a white suspension formed which was stiffed over the weekend. The reaction mixture was diluted with EtOAc (300 mL), and washed with water (100 mL). The aqueous layer was extracted with DCM (2×100 mL) and the combined organic layers were dried over MgSO₄, filtered and evaporated to afford crude product. The crude product was purified by flash silica chromatography elution gradient 0 to 100% EtOAc in DCM. Pure fractions were evaporated to dryness to afford (R)-tert-butyl 4-(5-hydroxypyrimidin-2-yl)-3-methylpiperazine-1-carboxylate (9.10 g, 81%) as a white solid. ¹H NMR (400.13 MHz, DMSO-d6) 1.05-1.07 (3H, m), 1.42 (9H, s), 2.75-3.15 (3H, m), 3.74-4.02 (3H, m), 4.32-4.40 (1H, m), 4.74-4.83 (1H, m), 6.63 (1H, t), 8.37 (2H, d). m/z (ES−) (M−H)−=279; HPLC tR=1.93 min.

INT 11

### (R)-Tert-butyl 4-(5-bromopyrimidin-2-yl)-3-methylpiperazine-1-carboxylate

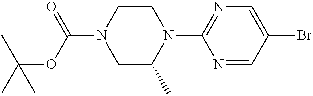

5-Bromo-2-chloropyrimidine (7.50 g, 38.77 mmol), (3R)-1-Boc-3-methylpiperazine (8.15 g, 40.71 mmol) and potassium carbonate (6.08 mL, 100.81 mmol) were suspended in butyronitrile (90 mL) and heated to 120° C. for 15 hours and cooled to RT. The solvent was removed in vacuo and the residue taken up in ethyl acetate (500 mL), washed with water (70 mL) and brine (70 mL), dried (sodium sulphate), filtered and evaporated to leave the crude product. The crude product was purified by flash silica chromatography elution gradient 0 to 20% EtOAc in DCM. Pure fractions were evaporated to dryness to afford (R)-tert-butyl 4-(5-bromopyrimidin-2-yl)-3-methylpiperazine-1-carboxylate (13.69 g, 99%) as a pale yellow gum which crystallised on standing. ¹H NMR (400 MHz, DMSO) 1.05 (d, 3H), 1.4 (s, 9H), 2.9 (m, 1H), 3.1 (m, 2H), 3.8 (d, 1H), 3.95 (m, 1H), 4.3 (d, 1H), 4.7 (m, 1H), 8.45 (s, 2H). m/z (ES+) (M+H)+=358; HPLC tR=3.55 min.

The following Bromides were prepared in a similar manner to Intermediate 11, using the appropriate piperazine and 5-bromo-2-chloropyrimidine:

| INTERMEDIATE | Name | ¹H NMR δ | MS |
|---|---|---|---|
| 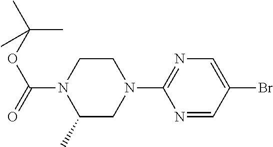 INT 12 | (S)-tcrt-butyl 4-(5- bromopyrimidin- 2-yl)-2- mcthylpipcrazine- 1-carboxylate | ¹H NMR (400.13 MHz, CDCl3) 1.13 (3H, d), 1.48 (9H, s), 2.94-3.04 (1H, m), 3.11-3.22 (2H, m), 3.91 (1H, dt), 4.29- 4.36 (1H, m), 4.41 (1H, dt), 4.46-4.52 (1H, m), 8.28 (2H, s) | m/z (ES+) (M − tBu)+ = 301.18; HPLC tR = 3.39 min. |
| 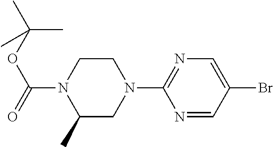 INT 13 | (R)-tcrt-butyl 4-(5- bromopyrimidin- 2-yl)-2- methylpiperazine- 1-carboxylate | 1H NMR(400.13 MHz, CDCl3) 1.13 (3H, d), 1.48 (9H, s), 2.94-3.04 (1H, m), 3.11-3.22 (2H, m), 3.91 (1H, dt), 4.29- 436 (1H, m), 4.41 (1H, dt), 4.46-4.52 (1H, m), 8.28 (2H, s) | m/z (ES+) (M − tBu)+ = 301.18; HPLC tR = 3.39 min. |
| 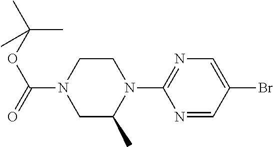 INT 14 | (S)-tcrt-butyl 4-(5- bromopyrimidin- 2-yl)-3- mcthylpipcrazine- 1-carboxylate | 1H NMR(CDCl3) 1.1 (d, 3H), 1.4 (s, 9H), 2.85 (br, 1H), 3.0-3.15 (m, 2H), 3.8-4.15 (m, 2H), 4.3 (br, 1H), 4.75 (br, 1H) and 8.25 (s, 2H). | m/z (ES+) (M − tBu)+ = 301; HPLC tR = 3.50 min. |
| 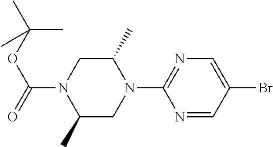 INT 15 | (2R,5S)-tcrt- butyl 4-(5- bromopyrimidin- 2-yl)-3,5- dimcthylpiperazine- 1-carboxylate | 1H NMR (400.132 MHz, CDCl3) 1.09-1.23 (6H, m), 1.48 (9H, s), 3.21- 3.37 (2H, m), 3.70- 3.85 (1H, m), 4.24-4.51 (2H, m), 4.77-4.87 (1H, m), 8.28 (2H, s) | m/z (ES+) (M − Boc) = 271, 273; HPLC tR = 3.54 min. |
| 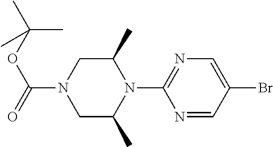 INT 16 | (3R,5S)-tert- butyl 4-(5- bromopyrimidin- 2-yl)-3,5- dimcthylpiperazine- 1-carboxylate | 1H NMR (400.13 MHz, CDCl3) 1.23 (6H, d), 1.50 (9H, s), 2.95-3.11 (2H, m), 3.91-4.14 (4H, m), 8.32 (2H, s) | m/z (ESI+) (M + H)+ = 373; HPLC tR = 3.69 min. |
| 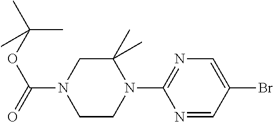 INT 17 | tert-butyl 4-(5- bromopyrimidin- 2-yl)-3,3- dimcthylpipcrazine- l-carboxylate | | m/z (ESI+) (M + H)+ = 373; HPLC tR = 3.77 min. |
| 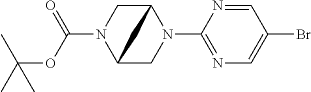 INT 18 | (1R,4R)-tert- butyl 5-(5- bromopyrimidin- 2-yl)-2,5- diazabicyclo[2.2.1] hcptanc-2- carboxylate | 1H NMR (400 MHz, DMSO) 1.35 (m, 9H), 1.9 (m, 2H), 3.15 (d, 1H), 3.35 (m, 2H), 3.5 (m, 1H), 4.45 (d, 1H), 4.8 (s, 1H), 8.4 (s, 2H). | m/z (ESI+) (M + H)+ = 357.06; HPLC tR = 3.13 min. |
| 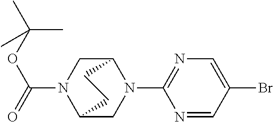 INT 19 | (1S,4S)-tcrt- butyl 5-(5- bromopyrimidin- 2-yl)-2,5- diazabicyclo[2.2.2] octane-2- carboxylate | ¹H NMR (400 MHz, CDCl₃) 1.4 (s, 9H), 1.7-1.8 (m, 2H), 1.9- 2.0 (m, 2H), 3.4-3.7 (m, 4H), 4.24.4 (d, 1H), 4.8 (d, 1H) and 8.2 (s, 2H). | m/z (ES+) (M − tBu)+ = 315; HPLC tR = 3.42 min. |

tert-butyl 4-(5-bromopyrimidin-2-yl)-piperazine-1-carboxylate is a known compound [WO2003010158]

The following aryl halides were prepared in a similar manner to Example 7 using Intermediate 1 and an appropriate alcohol.

| INTERMEDIATE | Name | 1H NMR δ | MS |
|---|---|---|---|
| 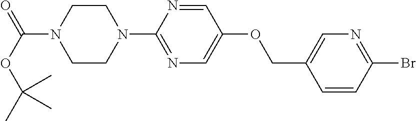 INT 20 | tert-butyl 4-(5- ((6-bromopyridin- 3-yl)methoxy) pyrimidin-2- yl)piperazine-l- carboxylate | 1H NMR (400.132 MHz, CDCl3) 1.48 (9H, s), 3.49 (4H, t), 3.72 (4H, t), 4.99 (2H, s), 7.52 (1H, d), 7.59- 7.64 (1H, m), 8.11 (2H, s), 8.38-8.42 (1H, m) | m/z (ES+) (M − Boc) = 350.28; HPLC tR = 3.37 min. |
| 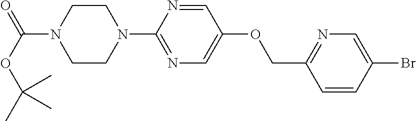 INT 21 | tert-butyl 4-(5- ((5-bromopyridin- 2-yl)mcthoxy) pyrimidin-2- yl)pipcrazine-1- carboxylate | | m/z (ES−) M− = 450; HPLC tR = 3.49 min. |
| 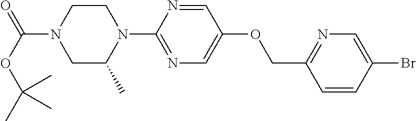 INT 22 | (R)-tcrt-butyl 4-(5-((5- bromopyridin-2- yl)methoxy) pyrimidin-2-yl)- 3-mcthylpipcrazine- l-carboxylate | 1H NMR (400.132 MHz, CDCl3) 1.15 (3H, d), 1.48 (9H, s), 2.81-2.99 (1H, m), 3.06-3.19 (2H, m), 3.74-4.01 (2H, m), 4.28- 4.35 (1H, m), 4.68-4.79 (1H, m), 5.10 (2H, s), 7.42 (1H, d), 7.84-7.88 (1H, m), 8.15 (2H, s), 8.66 (1H, d) | m/z (ES+) (M + H)+ = 464, 466; HPLC tR = 3.58 min. |
| 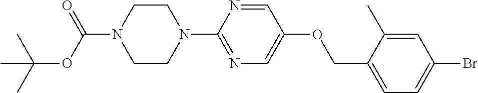 INT 23 | tert-butyl 4-(5- (4-bromo-2- methylbcnzyloxy) pyrimidin-2- yl)piperazine-l- carboxylate | 1H NMR (400.132 MHz, CDCl3) 1.48 (9H, s), 2.35 (3H, s), 3.47-3.51 (4H, m), 3.69-3.74 (4H, m), 4.95 (2H, s), 7.19-7.38 (3H, m), 8.10 (2H, s) | m/z (ES+) (M + H)+ = 463, 465; HPLC tR = 3.85 min. |
| 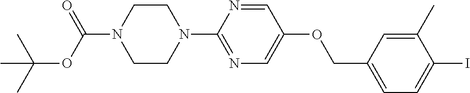 INT 24 | tert-butyl 4-(5- (4-iodo-3- methylbenzyloxy) pyrimidin-2- yl)piperazine-1- carboxylate | 1H NMR (400 MHz, DMSO) 1.4 (s, 9H), 2.35 (s, 3H), 3.35 (t, 4H), 3.6 (m, 4H), 5.0 (s, 2H), 7.0 (d, 1H), 7.4 (s, 1H), 7.8 (d, 1H), 8.25 (s, 2H). | m/z (ES+) (M + H)+ = 511.02; tR = 3.95 min. |
| 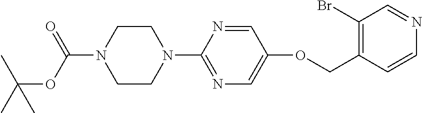 INT 25 | tert-butyl 4-(5- ((3-bromopyridin-4- yl)mcthoxy) pyrimidin-2- yl)pipcrazinc-l- carboxylate | 1H NMR (400.132 MHz, CDCl3) 1.48 (9H, s), 3.46- 3.52 (4H, m), 3.69-3.76 (4H, m), 5.07 (2H, s), 7.51 (1H, d), 8.15 (2H, s), 8.56 (1H, d), 8.72 (1H, s) | m/z (ES+) (M + H)+ = 450, 452; HPLC tR = 3.39 min. |

Intermediate 26

### (R)-tert-butyl 4-(5-((3-bromopyridin-4-yl)methoxy)pyrimidin-2-yl)-3-methylpiperazine-1-carboxylate

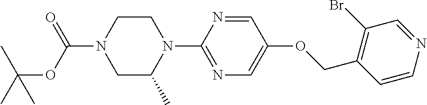

Diisopropyl azodicarboxylate (40.3 ml, 194.50 mmol) was added to (R)-tert-butyl 4-(5-hydroxypyrimidin-2-yl)-3-methylpiperazine-1-carboxylate (45.8 g, 155.60 mmol) (Intermediate 10) and triphenylphosphine (61.2 g, 233.40 mmol) in tetrahydrofuran (1000 ml) at 20° C. under nitrogen. The resulting solution was stirred at 20° C. for 30 minutes. (3-bromopyridin-4-yl)methanol (36.56 g, 194.45 mmol) was added. The resulting solution was stiffed overnight under nitrogen. The solvent was evaporated in vacuo to afford crude product.

The crude product was purified by flash silica chromatography, elution gradient 0 to 50% EtOAc in isohexane. Fractions were evaporated to dryness to afford (R)-tert-butyl 4-(5-((3-bromopyridin-4-yl)methoxy)pyrimidin-2-yl)-3-methylpiperazine-1-carboxylate (43.1 g, 60%) as a pale yellow gum. ¹H NMR (400.132 MHz, CDCl₃) 1.16 (3H, d), 1.48 (9H, s), 2.82-2.97 (1H, m), 3.08-3.21 (2H, m), 3.83-4.01 (2H, m), 4.29-4.40 (1H, m), 4.72-4.84 (1H, m), 5.06 (2H, s), 7.51 (1H, d), 8.16 (2H, s), 8.56 (1H, d), 8.72 (1H, s). m/z (ES+) (M+H)+=464,466; HPLC tR=3.47 min.

The following bromides were prepared in a similar manner to Example 1, using the appropriate hydroxypyrimidine and an appropriate bromide:

| INTERMEDIATE | Name | 1H NMR δ | MS |
|---|---|---|---|
| 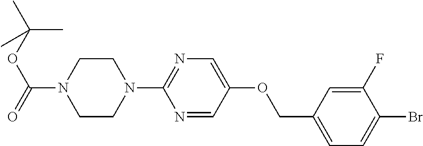 INT 27 | tcrt-butyl 4-(5- (4-bromo-3- fluorobcnzyloxy) pyrimidin-2- yl)pipcrazinc-l- carboxylate | 1H NMR (400.13 MHz, CDCl3) 1.48 (9H, s), 3.47- 3.50 (4H, m), 3.69-3.72 (4H, m), 4.97 (2H, s), 7.04- 7.07 (1H, m), 7.17-7.20 (1H, m), 7.54-7.57 (1H, m), 8.10 (2H, s) | m/z (ES+) (M + H)+ = 468.96; HPLC tR = 3.79 min. |
| 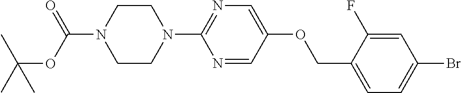 INT 28 | tcrt-butyl 4-(5- (4-bromo-2- fluorobenzyloxy) pyrimidin-2- yl)piperazine-l- carboxylate | 1H NMR (400 MHz, DMSO) 1.4 (s, 9H), 3.4 (m, 4H), 3.65 (m, 4H), 5.1 (s, 2H), 7.5 (m, 2H), 7.6 (d, 1H), 8.3 (s, 2H). | m/z (ES+) (M + H)+ = 468.96; HPLC tR = 3.88 min. |
| 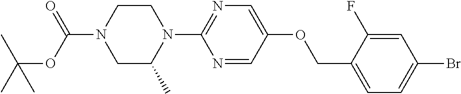 INT 29 | (R)-tcrt-butyl 4- (5-(4-bromo-2- fluorobcnzyloxy) pyrimidin-2-yl)-3- mcthylpipcrazine- 1-carboxylate | 1H NMR (400 MHz, DMSO) 1.0 (d, 3H), 1.4 (s, 9H), 2.85 (m, 1H), 3.0 (m, 2H), 3.8 (m, 1H), 3.95 (m, 1H), 4.2 (m, 1H), 4.7 (m, 1H), 5.1 (s, 2H), 7.5 (m, 2H), 7.6 (d, 1H), 8.25 (s, 2H). | m/z (ES+) (M + H)+ = 483.24; HPLC tR = 3.84 min. |
| 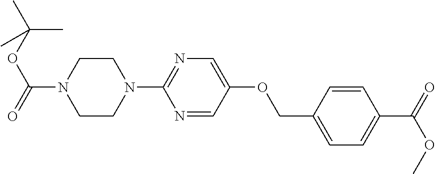 INT 30 | tert-butyl 4-(5- (4-(mcthoxy- carbonyl)benzyloxy) pyrimidin-2- yl)piperazine-1- carboxylate | 1H NMR (400.13 MHz, DMSO-d6) 1.41 (9H, s), 3.37 (4H, t), 3.58-3.61 (4H, m), 3.85 (3H, s), 5.19 (2H, s), 7.57 (2H, d), 7.97 (2H, d), 8.27 (2H, s) | m/z (ES+) (M + H)+ = 429.19; HPLC tR = 3.55 min. |
| 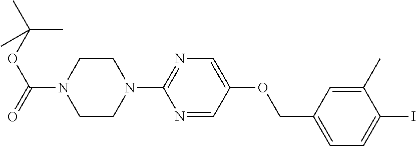 INT 31 | tcrt-butyl 4-(5- (4-iodo-3- mcthylbcnzyloxy) pyrimidin-2- yl)piperazine-1- carboxylate | 1H NMR (400.132 MHz, CDCl3) 1.48 (9H, s), 2.44 (3H, s), 3.48 (4H, t), 3.70 (4H, t), 4.93 (2H, s), 6.87- 6.92 (1H, m), 7.26-7.29 (1H, m), 7.81 (1H, d), 8.10 (2H, s) | m/z (ES+) (M + H)+ = 511.28; HPLC tR = 3.93 min. |
| 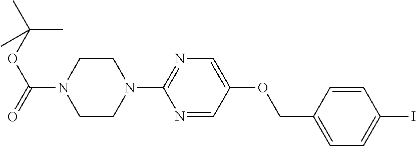 INT 32 | tcrt-butyl 4-(5- (4-iodobcnzyloxy) pyrimidin-2- yl)piperazine-1- carboxylate | 1H NMR (CDCl3) 1.4 (s, 9H), 3.4 (t, 4H), 3.6 (t, 4H), 4.9 (s, 2H),7.05 (d, 2H), 7.65 (d, 2H) and 8.0 (s, 2H). | m/z (ES+) (M + H)+ = 497; HPLC tR = 3.36 min. |

INT 33

### (R)-5-((3-Bromopyridin-4-yl)methoxy)-2-(2-methylpiperazin-1-yl)pyrimidine

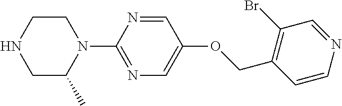

Hydrogen chloride (4M in dioxane) (17.01 mL, 68.05 mmol) was added to (R)-tert-butyl 4-(5-((3-bromopyridin-4-yl)methoxy)pyrimidin-2-yl)-3-methylpiperazine-1-carboxylate (3.16 g, 6.81 mmol) (Intermediate 26) in DCM (75 mL) at 20° C. The resulting solution was stirred at 20° C. for 30 minutes. The reaction was evaporated and azeotroped with toluene. Slurried in ether and evaporated to give (R)-5-((3-bromopyridin-4-yl)methoxy)-2-(2-methylpiperazin-1-yl)pyrimidine bis HCl salt (3.16 g, 100%) as a cream solid. ¹H NMR (400.13 MHz, DMSO-d6) 1.23 (2H, d), 2.85-2.96 (1H, m), 3.05-3.32 (4H, m), 4.45-4.52 (1H, m), 4.85-4.93 (1H, m), 5.21 (2H, s), 7.66 (1H, dd), 8.38 (2H, s), 8.62 (1H, d), 8.81 (1H, d), 9.15 (1H, d), 9.62 (1H, s).m/z (ES+) (M+H)+=366.31; HPLC tR=0.95 min.

The following amines were prepared in a similar manner to Intermediate 33, using an appropriate tert-butyl carbamate

| INTERMEDIATE | Name | 1H NMR δ | MS |
|---|---|---|---|
| 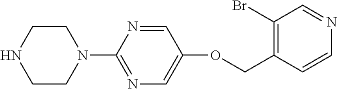 INT 34 | 5-((3- bromopyridin- 4-yl)methoxy)- 2-(piperazin-1- yl)pyrimidine | 1H NMR (400.132 MHz, DMSO) 3.09-3.15 (4H, m), 3.86-3.91 (4H, m), 5.22 (2H, s), 7.68 (1H, d), 8.38 (2H, s), 8.63 (1H, d), 8.82 (1H, s), 9.42 (1H, s) | m/z (ES+) (M + H)+ = 350, 352; HPLC tR = 0.95 min. |
| 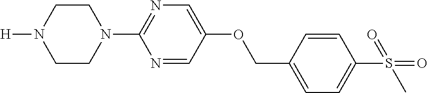 INT 35 | 5-(4- (mcthylsulfonyl) bcnzyloxy)- 2-(pipcrazin-l- yl)pyrimidine | 1H NMR (DMSO d6) 3.05 (t, 4H), 3.15 (s, 3H), 3.8 (t, 4H), 5.2 (s, 2H), 7.6 (d, 2H), 7.85 (d, 2H), 8.25 (s, 2H) and 9.3 (s, 2H). | m/z (ES+) (M + H)+ = 349.27; HPLC tR = 2.07 min |
| 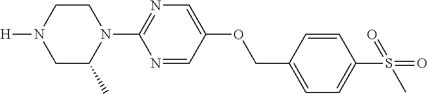 INT 36 | (R)-2-(2- mcthylpipcrazin- l-yl)-5-(4- (mcthylsulfonyl) benzyloxy) pyrimidine | 1H NMR (400.13 MHz, DMSO-d6) 1.24 (3H, d), 2.83-2.95 (1H, m), 3.03- 3.13 (1H, m), 3.14-3.31 (6H, m), 4.42-4.49 (1H, m), 4.83-4.92 (1H, m), 5.26 (2H, s), 7.70 (2H, d), 7.95 (2H, dt), 8.33 (2H, s), 9.18 (1H, s), 9.66 (1H, s) | m/z (ES+) (M + H)+ = 363.3; HPLC tR = 1.14 min. |
| 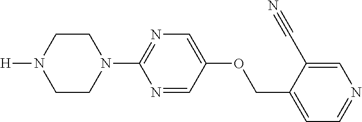 INT 64 | 4-[({2- [pipcrazin-1- yl]pyrimidin- 5-yl}oxy)methyl] pyridine-3- carbonitrilc | 1H NMR (400.132 MHz, DMSO) 3.11-3.17 (4H, m), 3.84-3.89 (4H, m), 5.36 (2H, s), 7.73 (1H, d), 8.38 (2H, s), 8.88 (1H, d), 9.06 (1H, s), 9.10 (1H, s) | m/z (ES+) (M + H)+ = 297; HPLC tR = 0.82 min. |
| 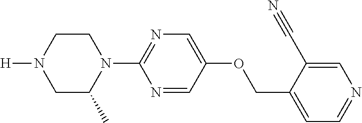 INT 65 | 4-[({2-[(2R)-2- mcthylpipcrazin- 1-yl]pyrimidin-5- yl}oxy)mcthyl] pyridinc-3- carbonitrilc | 1H NMR (400.13 MHz, DMSO-d6) 1.25 (3H, d), 2.85-2.97 (1H, m), 3.05- 3.32 (4H, m), 4.47 (1H, d), 4.85-4.93 (1H, m), 5.36 (2H, s), 7.74 (1H, dd), 8.38 (2H, s), 8.88 (1H, d), 9.05 (1H, s), 9.13 (1H, s), 9.57 (1H, s) | m/z (ES+) (M + H)+ = 311.37; HPLC tR = 0.78 min. |

INT 37

### 4-((2-(4-(tert-butoxycarbonyl)piperazin-1-yl)pyrimidin-5-yloxy)methyl)benzoic acid

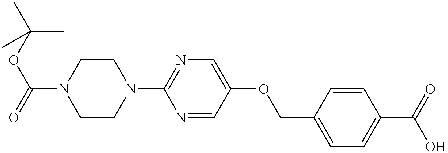

A solution of lithium hydroxide monohydrate (0.374 g, 8.92 mmol) in water (10 mL) was added to a stirred solution of tert-butyl 4-(5-(4-(methoxycarbonyl)benzyloxy)pyrimidin-2-yl)piperazine-1-carboxylate (1.91 g, 4.46 mmol) in THF (20 mL) at 20° C. The resulting mixture was stirred at 20° C. for 20 hours. Work up was carried out by diluting with water (50 mL) and then adjusting the pH to ˜4.5-5 using 1M HCl aq. The thick white suspension was extracted with DCM but not all of the solid would dissolve. The organics were separated and the aqueous was further extracted with DCM:MeOH (9:1) (3×100 mL). The combined organics were evaporated under reduced pressure then azeotroped with toluene (50 mL) to give 4-((2-(4-(tert-butoxycarbonyl)piperazin-1-yl)pyrimidin-5-yloxy)methyl)benzoic acid (1.920 g, 100%) as a white solid. ¹H NMR (400.13 MHz, DMSO-d6) 1.41 (9H, s), 3.35-3.39 (4H, m), 3.58-3.61 (4H, m), 5.18 (2H, s), 7.53 (2H, d), 7.94-7.96 (2H, m), 8.27 (2H, s), 12.97 (1H, br. s). m/z (ES+) (M+H)+=415.21; HPLC tR=1.96 min.

INT 38

### 4-((2-(4-(Tert-butoxycarbonyl)piperazin-1-yl)pyrimidin-5-yloxy)methyl)-2-methylbenzoic acid

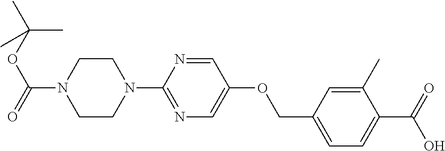

Tert-butyl 4-(5-(4-iodo-3-methylbenzyloxy)pyrimidin-2-yl)piperazine-1-carboxylate (600 mg, 1.18 mmol), trans-di-mu-acetatobis[2-(di-o-tolylphosphino)benzyl]dipalladium(II) (55.2 mg, 0.06 mmol), molybdenum hexacarbonyl (155 mg, 0.59 mmol) and N-ethyldiisopropylamine (0.410 mL, 2.35 mmol) were suspended in water (10 mL)/1,4-Dioxane (10 mL) and sealed into a microwave tube. The reaction was heated to 150° C. for 30 minutes in the microwave reactor and cooled to RT. The reaction mixture was concentrated and diluted with EtOAc (50 mL) this was extracted with saturated Na₂CO₃ (2×25 mL) the combined aqueous layer was acidified with 2M HCl. The aqueous layer was extracted with EtOAc (2×50 mL) and the organics washed with saturated brine (25 mL). The organic layer was dried over MgSO₄, filtered and evaporated to afford desired product. 4-((2-(4-(tert-butoxycarbonyl)piperazin-1-yl)pyrimidin-5-yloxy)methyl)-2-methylbenzoic acid (289 mg, 57%) as a pale brown solid. ¹H NMR (400.132 MHz, CDCl₃) 1.48 (9H, s), 2.66 (3H, s), 3.48 (4H, t), 3.71 (4H, t), 5.04 (2H, s), 7.28-7.32 (2H, m), 8.05 (1H, d), 8.12 (2H, s). m/z (ES−) (M−H)−=427.45; HPLC tR=3.38 min.

INT 39

### Phenyl 1,1,1-trifluoropropan-2-yl carbonate

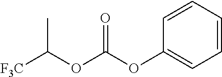

Phenyl chloroformate (2.424 mL, 19.29 mmol) was added to 1,1,1-trifluoro-2-propanol (2.0 g, 17.53 mmol) in pyridine (20 mL) at 0° C. over a period of 5 minutes under nitrogen. The resulting suspension was stirred and allowed to warm to ambient temperature over 4 days. The pyridine was removed in vacuo keeping the water bath temperature below 40° C. The residue was taken up in DCM (200 mL) and washed with saturated aqueous sodium bicarbonate (100 mL), dried (magnesium sulphate) and concentrated in vacuo. This gave phenyl 1,1,1-trifluoropropan-2-yl carbonate (3.06 g, 74%) as a straw coloured oil. ¹H NMR (400 MHz, DMSO) d 1.5 (t, 3H), 5.45 (m, 1H), 7.4 (m, 5H).

m/z (ES−) (M−H)−=233.02; HPLC tR=3.00 min.

INT 40

4-Fluorophenyl 3-methyloxetan-3-yl carbonate

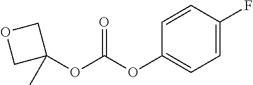

4-Fluorophenyl carbonochloridate (0.169 mL, 1.29 mmol) was added to 3-methyloxetan-3-ol (0.63 g, 1.29 mmol) at 0° C. under nitrogen. DCM (2 mL) was added to the resulting slurry and the suspension formed was stirred and allowed to warm to ambient temperature and stirred for 3 hours. The reaction mixture was evaporated to dryness and redissolved in chloroform (20 mL) and washed with 1M citric acid (15 mL). The organic layer was filtered through a phase separation tube and evaporated to afford desired product which was used without purification. Contains 4-fluorophenol as an impurity. ¹H NMR (400.132 MHz, CDCl₃) 1.82 (3H, s), 4.53 (2H, d), 4.87 (2H, d), 7.06-7.18 (4H, m)

INT 41

### 3-(4-(5-((3-Bromopyridin-4-yl)methoxy)pyrimidin-2-yl)piperazin-1-yl)-5-isopropyl-1,2,4-oxadiazole

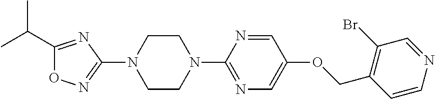

(E)-N-((4-(5-((3-bromopyridin-4-yl)methoxy)pyrimidin-2-yl)piperazin-1-yl)(hydroxyimino)methyl)isobutyramide (Intermediate 43) (260 mg, 0.33 mmol) was suspended in toluene (80 mL) and stirred at 120° C. for 30 minutes. It was cooled to room temperature and concentrated in vacuo. The crude product was purified by flash silica chromatography, elution gradient 10 to 60% EtOAc in DCM. Pure fractions were evaporated to dryness to afford 3-(4-(5-((3-bromopyridin-4-yl)methoxy)pyrimidin-2-yl)piperazin-1-yl)-5-isopropyl-1,2,4-oxadiazole (140 mg, 93%) as a white solid. ¹H NMR (400.132 MHz, CDCl₃) 1.36 (6H, d), 3.09 (1H, septet), 3.50-3.54 (4H, m), 3.84-3.88 (4H, m), 5.08 (2H, s), 7.51 (1H, d), 8.17 (2H, s), 8.56 (1H, d), 8.72 (1H, s).m/z (ES+) (M+H)+=460, 462; HPLC tR=2.66 min.

Intermediate 42

### (R)-3-(4-(5-((3-bromopyridin-4-yl)methoxy)pyrimidin-2-yl)-3-methylpiperazin-1-yl)-5-isopropyl-1,2,4-oxadiazole

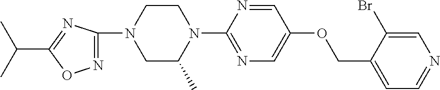

Hydroxylamine hydrochloride (22.12 g, 318.3 mmol) was added portionwise to (R)-4-(5-((3-bromopyridin-4-yl)methoxy)pyrimidin-2-yl)-3-methylpiperazine-1-carbonitrile (88.5 g, 227.4 mmol, INT 54) in dioxane (700 ml) (which had been dried over sieves) and N-Ethyldiisopropylamine (55.1 ml, 318.3 mmol), which was stirred under nitrogen at 50° C. The resulting mixture was then heated and stirred at 80° C. for 2 hours, and a white suspension was formed. The reaction was cooled to 25° C., and to the reaction mixture was added 300 ml of water and pyridine (73.4 ml, 909.4 mmol), this was followed by a steady addition of isobutyryl chloride (45.1 ml, 432 mmol) over 20 mins, controlling the exotherm, and not allowing it to go above 30° C. Once addition was complete, the reaction was allowed to stir overnight at room temperature. Ethyl acetate (1000 ml) was added, and 300 ml of water, and an extraction carried out. The organics were collected, evaporated to dryness and purified by silica chromatography, with graduated solvent 0-50% ethyl acetate/isohexane. Appropriate fractions were collected and evaporated to dryness yielding a pale yellow gum, which crystallised on standing. The product was triturated with a small amount of diethyl ether, and air dried yielding (R)-3-(4-(5-((3-bromopyridin-4-yl)methoxy)pyrimidin-2-yl)-3-methylpiperazin-1-yl)-5-isopropyl-1,2,4-oxadiazole (61.5 g, 57%) as a white solid. ¹H NMR (400.13 MHz, CDCl₃) 1.17 (3H, d), 1.28 (6H, d), 2.93-3.07 (2H, m), 3.15 (1H, dd), 3.24 (1H, ddd), 3.78 (1H, dt), 3.94 (1H, d5), 4.39 (1H, dq), 4.79-4.87 (1H, m), 5.00 (2H, d), 7.45 (1H, dd), 8.10 (2H, s), 8.49 (1H, d), 8.63 (1H, s). m/z (ES+) (M+H)+=476.42; HPLC tR=2.78 min

INT 43

### (E)-N-((4-(5-((3-Bromopyridin-4-yl)methoxy)pyrimidin-2-yl)piperazin-1-yl)(hydroxyimino)methyl)isobutyramide

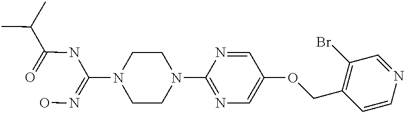

N-Ethyldiisopropylamine (0.114 mL, 0.66 mmol) was added to (E)-4-(5-((3-bromopyridin-4-yl)methoxy)pyrimidin-2-yl)-N′-hydroxypiperazine-1-carboximidamide (0.27 g, 0.66 mmol), isobutyric acid (0.061 mL, 0.66 mmol) and 1-Hydroxybenzotriazole (0.098 g, 0.73 mmol) in DMF (4 mL). The resulting solution was stiffed at 20° C. for 10 minutes. 1-(3-dimethylaminopropyl)-3-ethylcarbodiimide hydrochloride (0.153 g, 0.80 mmol) was added and the resulting solution stirred at 20° C. for 1 hour. It was partitioned between 50% brine (50 mL) and ethyl acetate (2×100 mL) and the combined organics washed with saturated aqueous sodium bicarbonate (50 mL), water (50 mL) and brine (50 mL), dried (sodium sulphate) and concentrated in vacuo. This gave (E)-N-((4-(5-((3-bromopyridin-4-yl)methoxy)pyrimidin-2-yl)piperazin-1-yl)(hydroxyimino)methyl)isobutyramide (0.260 g, 82%) as a pale yellow gum. The mixture also contained the primary amide contaminant from the starting material. This was taken forward as crude material and purified at the next step. m/z (ES+) (M+H)+=478, 480; HPLC tR=1.59 min.

The following Intermediates were prepared in a similar manner to Intermediate 43, using the intermediate starting material (Int sm) shown and the appropriate carboxylic acid:

| Structure | Int Sm | Name | 1H NMR δ | MS |
|---|---|---|---|---|
| 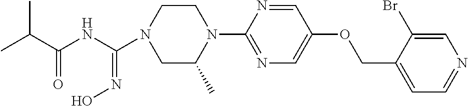 INT 44 | 50 | (R,E)-N-((4-(5-((3- bromopyridin-4- yl)methoxy) pyrimidin-2-yl)-3- methylpiperazin-1- yl)(hydroxyimino) mcthyl)isobutyramidc | | |
| 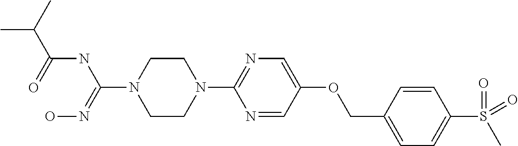 INT 45 | 51 | (E)-N-((hydroxyimino) (4-(5-(4- (mcthylsulfonyl) bcnzyloxy)pyrimidin- 2-yl)pipcrazin-l- yl)mcthyl) isobutyramide | | m/z (ES+) (M + H)+ = 477.27; HPLC tR = 1.55 min |
| 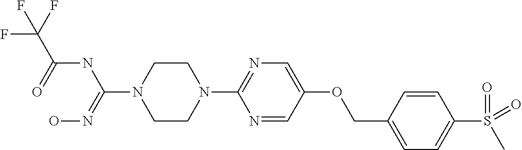 INT 46 | 51 | (E)-2,2,2-trifluoro-N- ((hydroxyimino)(4-(5- (4-(mcthylsulfonyl) benzyloxy)pyrimidin-2- yl)pipcrazin-l-yl) methyl)acetamide | | m/z (ES+) (M + H)+ = 491.44; HPLC tR = 1.59 min. |
| 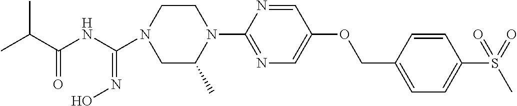 INT 47 | 52 | (R,E)-N- ((hydroxyimino)(3- mcthyl-4-(5-(4- (mcthylsulfonyl) benzyloxy)pyrimidin- 2-yl)pipcrazin-l- yl)mcthyl) isobutyramide | | |
| 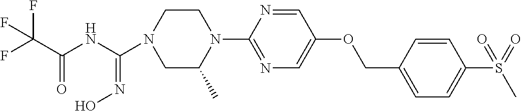 INT 48 | 52 | (R,E)-2,2,2-trifluoro- N-((hydroxyimino)(3- mcthyl-4-(5-(4- (mcthylsulfonyl) benzyloxy)pyrimidin- 2-yl)pipcrazin-l- yl)mcthyl)acetamide | | |

INT 49

### (E)-4-(5-((3-bromopyridin-4-yl)methoxy)pyrimidin-2-yl)-N′-hydroxypiperazine-1-carboximidamide

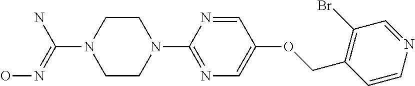

Potassium carbonate (0.250 g, 1.81 mmol) was added to 4-(5-((3-bromopyridin-4-yl)methoxy)pyrimidin-2-yl)piperazine-1-carbonitrile (Intermediate 53) (0.68 g, 1.81 mmol) and hydroxylamine hydrochloride (0.252 g, 3.62 mmol) in ethanol (10 mL) and water (15 mL). The resulting solution was stiffed at 85° C. for 1 hour. It was cooled to room temperature and the ethanol removed in vacuo. A precipitate formed in the remaining aqueous solution and this was filtered, washed with water, collected and azeotroped once with toluene and dried under vacuum to give (E)-4-(5-((3-bromopyridin-4-yl)methoxy)-pyrimidin-2-yl)-N′-hydroxypiperazine-1-carboximidamide (0.550 g, 74%) as a white solid. ¹H NMR (400.132 MHz, DMSO) 3.01-3.06 (4H, m), 3.61-3.65 (4H, m), 5.16-5.19 (4H, m), 6.01 (1H, s), 7.61 (1H, d), 8.30 (2H, s), 8.59 (1H, d), 8.76 (1H, s). m/z (ES+) (M+H)+=408, 410; HPLC tR=1.24 min.

This is about 65% pure with the impurity being 4-{5-[3-bromopyridin-4-yl)methoxy]pyrimidin-2-yl}piperazine-1-carboxamide. This was carried through and removed at the final step.

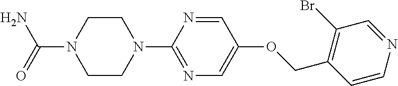

¹H NMR (400.132 MHz, DMSO) 3.33-3.37 (4H, m), 3.58-3.61 (4H, m), 5.17 (2H, s), 6.00 (2H, s), 7.60 (1H, d), 8.31 (2H, s), 8.58 (1H, d), 8.75 (1H, s). m/z (ES+) (M+H)+=393, 395; HPLC tR=1.45 min.

The following Intermediates were prepared in a similar manner to Intermediate 43 using the intermediate nitriles (int sm) listed and hydroxylamine:

| Structure | int sm | Name | 1H NMR δ | MS |
|---|---|---|---|---|
| 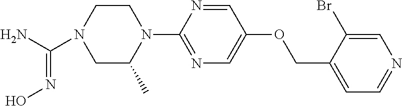 INT 50 | 54 | (R,E)-4-(5-((3- bromopyridin-4- yl)mcthoxy)pyridin- 2-yl)-N′-hydroxy-3- methylpiperazine-1- carboximidamide | | m/z (ES+) (M + H)+ = 424.32; HPLC tR = 1.06 min. |
| 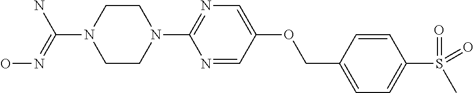 INT 51 | 55 | (E)-N′-hydroxy-4- (5-(4-(methylsulfonyl) bcnzyloxy)pyrimidin- 2-yl)piperazine-1- carboximidamide | | m/z (ES+) (M + H)+ = 407.35; HPLC tR = 1.04 min. |
| 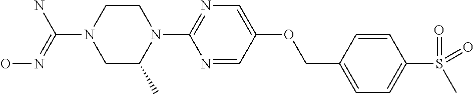 INT 52 | 56 | (R,E)-N′-hydroxy-3- mcthyl-4-(5-(4- (methylsulfonyl) benzyloxy)pyrimidin- 2-yl)piperazine-1- carboximidamide | | m/z (ES+) (M + H)+ = 421.46; HPLC tR = 1.04 min. |

INT 53

### 4-(5-((3-Bromopyridin-4-yl)methoxy)pyrimidin-2-yl)piperazine-1-carbonitrile

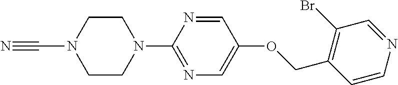

A solution of cyanogen bromide (0.921 g, 8.70 mmol) in dichloromethane (10.00 mL) was added slowly to a stirred suspension of 5-((3-bromopyridin-4-yl)methoxy)-2-(piperazin-1-yl)pyrimidine (1.84 g, 4.35 mmol) and triethylamine (3.64 mL, 26.09 mmol) in dichloromethane (20 mL) cooled to 0° C., over a period of 5 minutes. The resulting suspension was stirred at ambient temperature for 2 hours. The reaction mixture was diluted with DCM (150 mL), and washed with saturated sodium bicarbonate solution (20 mL). The organic layer was dried over MgSO₄, filtered and evaporated to afford crude product. The crude product was purified by flash silica chromatography elution gradient 0 to 60% EtOAc in DCM. Pure fractions were evaporated to dryness to afford 4-(5-((3-bromopyridin-4-yl)methoxy)pyrimidin-2-yl)piperazine-1-carbonitrile (0.680 g, 42%) as a white solid. ¹H NMR (400.132 MHz, CDCl₃) 3.27-3.32 (4H, m), 3.85-3.89 (4H, m), 5.08 (2H, s), 7.50 (1H, d), 8.16 (2H, s), 8.56 (1H, d), 8.72 (1H, s). m/z (ES+) (M+H)+=375, 377; HPLC tR=1.92 min.

Intermediate 54

### (R)-4-(5-((3-bromopyridin-4-yl)methoxy)pyrimidin-2-yl)-3-methylpiperazine-1-carbonitrile

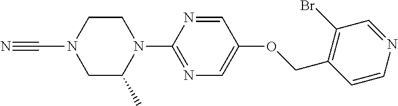

A slurry of sodium bicarbonate (92 g, 1098 mmol) in water (360 mL) and (R)-5-((3-bromopyridin-4-yl)methoxy)-2-(2-methylpiperazin-1-yl)pyrimidine dihydrochloride (120 g, 219.6 mmol, INT 33) in DCM (1200 ml) was stirred at 0° C. Cyanogen bromide (25.5 ml, 263.5 mmol) was added as a solid portionwise over 5 mins and the resulting suspension stiffed at 0° C. for 30 minutes. It was then stirred at 20° C. for 16 hours. Another 0.25 equivs of cyanogen bromide was added and allowed to stir for 2 hrs. The organic layer was separated, and washed with 100 ml of sodium bicarbonate solution. The organics were then evaporated to dryness yielding crude product which was purified by flash silica chromatography, elution gradient 0 to 50% EtOAc in DCM. The resultant gum was triturated with diethyl ether to give (R)-4-(5-((3-bromopyridin-4-yl)methoxy)pyrimidin-2-yl)-3-methylpiperazine-1-carbonitrile (42.0 g, 49%) as a cream solid. ¹H NMR (400.13 MHz, DMSO-d6) 1.17 (3H, m), 3.07-3.20 (2H, m), 3.41-3.50 (1H, m), 4.28-4.37 (1H, m), 4.73-4.81 (1H, m), 5.17 (2H, s), 7.60 (1H, dd), 8.34 (2H, s), 8.58 (1H, d), 8.75 (1H, s). m/z (ES+) (M+H)+=391.29; HPLC tR=2.04 min.

The following Intermediates were prepared in a similar manner to Intermediate 53 using the intermediate shown and cyanogen bromide (Intsm=intermediate starting material):

| Structure | Int Sm | Name | 1H NMR δ | MS |
|---|---|---|---|---|
| 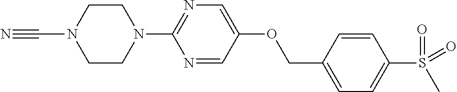 INT 55 | 35 | 4-(5-(4- (methylsulfonyl) bcnzyloxy) pyrimidin-2- yl)pipcrazine- 1-carbonitrile | 1H NMR (400.13 MHz, CDCl3) δ 3.06 (3H, s), 3.27-3.30 (4H, m), 3.86 (4H, t), 5.13 (2H, s), 7.62 (2H, d), 7.96-7.99 (2H, m), 8.13 (2H, s) | m/z (ES+) (M + H)+ = 374.14; HPLC tR = 2.33 min. |
| 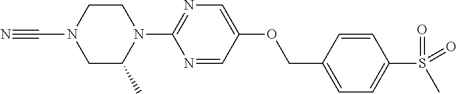 INT 56 | 37 | (R)-3-methyl- 4-(5-(4- (mcthylsulfonyl) bcnzyloxy) pyrimidin-2- yl)piperazine- 1-carbonitrile | 1H NMR (400.13 MHz, DMSO-d6) 1.18 (3H, d), 3.04-3.19 (2H, m), 3.21 (3H, s), 3.28 (2H, d), 3.40- 3.49 (1H, m), 4.26-4.35 (1H, m), 4.71-4.79 (1H, m), 5.24 (2H, s), 7.69 (2H, d), 7.95 (2H, dt), 8.31 (2H, s) | m/z (ES+) (M + H)+ = 388.36; HPLC tR = 1.95 min. |

INT 57

### Tert-butyl 4-(5-(4-(trifluoromethylthio)benzyloxy)pyrimidin-2-yl)piperazine-1-carboxylate

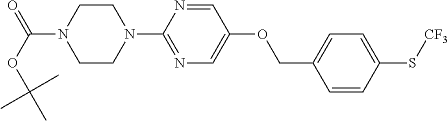

To a stirred suspension of tert-butyl 4-(5-hydroxypyrimidin-2-yl)piperazine-1-carboxylate (0.981 g, 3.5 mmol) and (4-(bromomethyl)phenyl)(trifluoromethyl)sulfane (0.996 g, 3.68 mmol) in DMF (10.90 mL) at ambient temperature was added cesium carbonate (3.42 g, 10.50 mmol). The mixture was stiffed at ambient temperature for 16 hours, poured onto water (165 mL), extracted with ethyl acetate (3×60 mL), the combined ethyl acetate extracts washed with brine, dried (MgSO₄) and evaporated in vacuo to a residue which was crystallised from ethyl acetate/isohexane to give tert-butyl 4-(5-(4-(trifluoromethylthio)benzyloxy)pyrimidin-2-yl)piperazine-1-carboxylate (1.430 g, 87%). ¹H NMR (CDCl₃) 1.5 (s, 9H), 3.5 (t, 4H), 3.7 (t, 4H), 5.05 (s, 2H), 7.45 (d, 2H), 7.7 (d, 2H) and 8.1 (s, 2H). m/z (ES) (M+H)+=471; HPLC tR=3.50 min.

INT 58

### Tert-butyl 4-(5-(4-(2-morpholinoethylthio)benzyloxy)pyrimidin-2-yl)piperazine-1-carboxylate

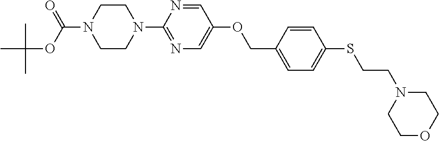

Potassium carbonate (0.553 mL, 9.16 mmol) was added to (4-mercaptophenyl)methanol (0.584 g, 4.17 mmol) and 4-(2-chloroethyl)morpholine hydrochloride (0.930 g, 5.00 mmol) in DMF (10 mL) at 40° C. The resulting solution was stirred at 40° C. for 12 hours. The reaction mixture was diluted with EtOAc (50 mL), and washed sequentially with saturated brine (3×50 mL). The organic layer was dried over MgSO₄, filtered and evaporated to afford crude (4-(2-morpholinoethylthio)phenyl)methanol (1.27 g, 5.01 mmol). Tert-butyl 4-(5-hydroxypyrimidin-2-yl)piperazine-1-carboxylate (1.405 g, 5.01 mmol) and triphenylphosphine (1.643 g, 6.27 mmol) in tetrahydrofuran (82 mL) was added then diisopropyl azodicarboxylate (1.299 mL, 6.27 mmol). The mixture was stirred at 50° C. for 16 hours, the tetrahydrofuran evaporated in vacuo to a residue. The crude product was purified by flash silica chromatography, elution gradient 0 to 20% MeOH in DCM. The crude solid was triturated with Et₂O to give a solid which was collected by filtration and dried under vacuum to give tert-butyl 4-(5-(4-(2-morpholinoethylthio)benzyloxy)-pyrimidin-2-yl)piperazine-1-carboxylate (0.910 g, 35%) as a white solid. ¹H NMR (400.13 MHz, CDCl₃) 1.48 (9H, s), 2.48 (4H, t), 2.62-2.65 (2H, m), 3.04-3.08 (2H, m), 3.47-3.49 (4H, m), 3.68-3.72 (8H, m), 4.98 (2H, s), 7.29-7.36 (4H, m), 8.10 (2H, s). m/z (ES+) (M+H)+=516; HPLC tR=1.54 min.

INT 59

### tert-butyl 4-(5-(4-(2-morpholinoethylsulfinyl)benzyloxy)pyrimidin-2-yl)piperazine-1-carboxylate

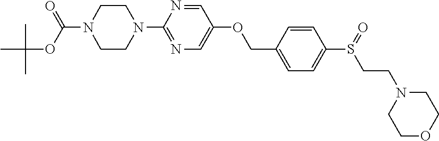

To a stirred solution of tert-butyl 4-(5-(4-(2-morpholinoethylthio)benzyloxy)pyrimidin-2-yl)piperazine-1-carboxylate (0.291 g, 0.56 mmol) in methanol (0.673 mL) was added a solution of sodium tungstate dihydrate (3.72 mg, 0.01 mmol) in water (0.013 mL). The mixture was heated to 55° C. and treated with hydrogen peroxide (0.070 mL, 1.13 mmol) over 1 minute. When the addition was completed, the mixture was heated at 55° C. for 30 minutes, cooled to ambient temperature, treated with saturated sodium hydrogen carbonate solution (12 mL), the methanol evaporated in vacuo and the aqueous residue extracted with ethyl acetate (3×125 mL). The combined ethyl acetate extracts were dried (MgSO₄) and evaporated. The crude product was purified by flash silica chromatography, elution gradient 1 to 5% MeOH in DCM. Pure fractions were evaporated to dryness to afford tert-butyl 4-(5-(4-(2-morpholinoethylsulfinyl)benzyloxy)pyrimidin-2-yl)piperazine-1-carboxylate (0.335 g, 100%) as a white solid. m/z (ES+) (M+H)+=532; HPLC tR=1.41 min.

INT 60

### Tert-butyl 4-(5-(4-(methylthio)benzyloxy)pyrimidin-2-yl)piperazine-1-carboxylate

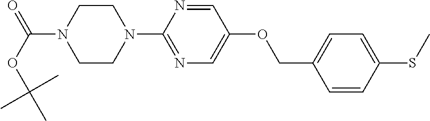

Diisopropyl azodicarboxylate (0.924 mL, 4.46 mmol) was added to tert-butyl 4-(5-hydroxypyrimidin-2-yl)piperazine-1-carboxylate (1 g, 3.57 mmol) and triphenylphosphine (1.403 g, 5.35 mmol) in tetrahydrofuran (30 mL) at 20° C. under nitrogen. The resulting solution was stiffed at 20° C. for 30 minutes then (4-(methylthio)phenyl)methanol (0.688 g, 4.46 mmol) was added. The resulting solution was stirred at rt overnight under nitrogen. The solvent was evaporated and the residue diluted with EtOAc and brine. The aqueous layer was extracted with EtOAc (50 mL) and the combined organics were concentrated in vacuo to afford crude product. The crude product was purified by flash silica chromatography, elution gradient 1 to 4% MeOH in DCM. The crude product was re-purified by flash silica chromatography, elution gradient 10 to 20% EtOAc in isohexane. Pure fractions were evaporated to dryness to afford tert-butyl 4-(5-(4-(methylthio)-benzyloxy)pyrimidin-2-yl)piperazine-1-carboxylate (0.500 g, 34%) as a white solid. ¹H NMR (400.132 MHz, CDCl₃) 1.47 (9H, s), 2.48 (3H, s), 3.46-3.51 (4H, m), 3.67-3.72 (4H, m), 4.97 (2H, s), 7.25-7.32 (4H, m), 8.10 (2H, s). m/z (ES+) (M+H)+=417; HPLC tR=3.68 min.

INT 61

Tert-butyl 4-(5-((2-chloropyrimidin-5-yl)methoxy)pyrimidin-2-yl)piperazine-1-carboxylate

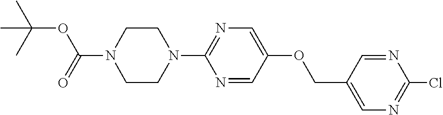

Diisopropyl azodicarboxylate (0.140 mL, 0.71 mmol) was added to a stirred solution of tert-butyl 4-(5-hydroxypyrimidin-2-yl)piperazine-1-carboxylate (0.16 g, 0.57 mmol), and triphenylphosphine (0.225 g, 0.86 mmol) in THF (5 mL) under nitrogen. The resulting solution was stiffed at 20° C. for 30 minutes and then (2-chloropyrimidin-5-yl)methanol (0.083 g, 0.57 mmol) was added. The resulting solution was stirred at rt for 24 hours under nitrogen. The solvent was evaporated and the residue diluted with EtOAc and brine. A white ppt was filtered off and dried under vacuum. The aqueous layer was extracted with EtOAc (50 mL) and the combined organics were concentrated in vacuo to afford crude product. The crude product was purified by flash silica chromatography, elution gradient 1 to 4% MeOH in DCM. The crude product was purified by flash silica chromatography, elution gradient 40 to 100% EtOAc in isohexane. Pure fractions were evaporated to dryness to afford tert-butyl 4-(5-((2-chloropyrimidin-5-yl)methoxy)pyrimidin-2-yl)piperazine-1-carboxylate (0.100 g, 43%) as a white solid. ¹H NMR (400.132 MHz, CDCl3) 1.49 (9H, s), 3.47-3.52 (4H, m), 3.71-3.75 (4H, m), 5.02 (2H, s), 8.14 (2H, s), 8.69 (2H, s). m/z (ES+) (M+H)+=407; HPLC tR=3.18 min.

INT 62

### Tert-butyl 4-(5-((2-aminopyrimidin-5-yl)methoxy)pyrimidin-2-yl)piperazine-1-carboxylate

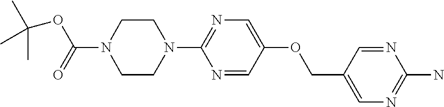

Tert-butyl 4-(5-((2-chloropyrimidin-5-yl)methoxy)pyrimidin-2-yl)piperazine-1-carboxylate (0.1 g, 0.25 mmol) and ammonia (0.5M in dioxane) (15 mL, 7.50 mmol) were sealed into a microwave tube. The reaction was heated to 130° C. for 5 hours in the microwave reactor and cooled to RT. The reaction was incomplete so the temperature was increased to 140° C. and the reaction mixture was stirred for a further 3 hours and then a further 9 hours. The reaction mixture was evaporated to dryness and redissolved in EtOAc (25 mL), and washed sequentially with water (20 mL) and saturated brine (20 mL). The organic layer was dried over Na₂SO₄, filtered and evaporated to afford desired product, tert-butyl 4-(5-((2-aminopyrimidin-5-yl)methoxy)pyrimidin-2-yl)piperazine-1-carboxylate (0.100 g, 100%) as a yellow solid. 50:50 mixture of NH2 and starting chloro. Used crude in the next step. m/z (ES+) (M+H)+=388; HPLC tR=2.77 min.

INT 63

### 4-(Chloromethyl)-N-(2-hydroxyethyl)-N-methylbenzamide

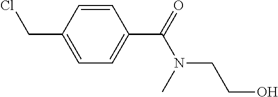

To a stirred solution of 4-(chloromethyl)benzoic acid (5.00 g, 29.31 mmol) in dichloromethane (136 mL) was added oxalyl chloride (3.08 mL, 35.17 mmol) and 1 drop of DMF and the mixture stirred at ambient temperature for 16 hours. The dichloromethane was evaporated in vacuo to a residue which was taken up in dichloromethane (20 mL) and added at 0° C. to a stirred solution of 2-(methylamino)ethanol (7.06 mL, 87.93 mmol) in dichloromethane (70 mL). When the addition was completed, the mixture was allowed to come to ambient temperature and stirred for 2 hours. The dichloromethane was evaporated in vacuo to a residue which was partitioned between water (50 mL) and diethyl ether (125 mL), the diethyl ether layer washed with 1M hydrochloric acid, saturated sodium hydrogen carbonate solution, brine, dried (MgSO₄) and evaporated in vacuo to give 4-(chloromethyl)-N-(2-hydroxyethyl)-N-methylbenzamide (0.360 g, 5%). ¹H NMR (CDCl₃) 2.1 (s, 1H), 3.0 (s, 3H), 3.4-3.9 (m, 4H), 4.5 (s, 2H), and 7.4 (s, 4H).

INT 64 and INT 65

See after INT 36.There is no INT 66.

INT 67

### 4-(chloromethyl)nicotinonitrile

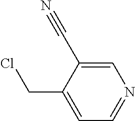

Benzoyl peroxide (0.041 g, 0.17 mmol) was added to 4-methylnicotinonitrile (2 g, 16.93 mmol) and sulfuryl chloride (2.72 mL, 33.86 mmol) in carbon tetrachloride (40 mL) at 20° C. The resulting suspension was stirred at 80° C. for 3 hours then cooled and DCM (100 mL) added. The mixture was washed with water (150 mL) and brine (100 mL). The organic layer was filtered through a phase separation tube and evaporated to give 4-(chloromethyl)nicotinonitrile (1.590 g, 61.6%) as a brown oil. 42% pure by LCMS and NMR with the rest unreacted starting material ¹H NMR (400 MHz, CDCl₃) δ 4.72 (2H, s), 7.60 (1H, d), 8.84 (1H, d), 8.89 (1H, s). m/z (ES+) (M+H)+=153.17; HPLC tR=1.70 min.

INT 68

### (R)-3-(4-(5-((3-bromopyridin-4-yl)methoxy)pyrimidin-2-yl)-3-methylpiperazin-1-yl)-5-cyclopropyl-1,2,4-oxadiazole

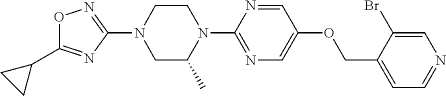

N-Ethyldiisopropylamine (0.557 mL, 3.22 mmol) was added to (R,E)-4-(5-((3-bromopyridin-4-yl)methoxy)pyrimidin-2-yl)-N′-hydroxy-3-methylpiperazine-1-carboximidamide (618 mg, 1.46 mmol, INT 50), cyclopropanecarboxylic acid (0.232 mL, 2.93 mmol) and 1-hydroxybenzotriazole (435 mg, 3.22 mmol) in DMF (12 mL) under nitrogen. 1-(3-Dimethylaminopropyl)-3-ethylcarbodiimide hydrochloride (673 mg, 3.51 mmol) was added and the resulting suspension was stirred at 20° C. for 18 hours. It was diluted with ethyl acetate (200 mL) and washed with water (4×80 mL) and brine (80 mL), dried (magnesium sulphate) and concentrated in vacuo. The residue was suspended in toluene (35 mL). The resulting mixture was stirred at 120° C. for 1 hour then cooled to room temperature and concentrated in vacuo then taken up in DCM and adsorbed onto silica. The crude product was purified by flash silica chromatography, elution gradient 0 to 100% EtOAc in isohexane. Pure fractions were evaporated to dryness to afford (R)-3-(4-(5-((3-bromopyridin-4-yl)methoxy)pyrimidin-2-yl)-3-methylpiperazin-1-yl)-5-cyclopropyl-1,2,4-oxadiazole (383 mg, 55%) as a pale yellow solid. ¹H NMR (400.13 MHz, DMSO-d6) 1.1 (7H, m), 2.15 (1H, m), 2.95 (1H, m), 3.15 (2H, m), 3.7 (1H, d), 3.85 (1H, d), 4.35 (1H, d), 4.8 (1H, m), 5.15 (2H, s), 7.6 (1H, d), 8.3 (2H, s), 8.6 (1H, d), 8.75 (1H, s). m/z (ES+) (M+H)+=474.21; HPLC tR=2.77 min

INT 69

### (R)-2-(4-(3-isopropyl-1,2,4-oxadiazol-5-yl)-2-methylpiperazin-1-yl)pyrimidin-5-ol

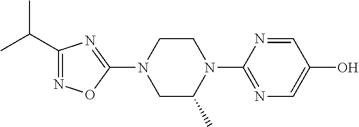

A solution of zinc chloride (24.08 ml, 24.08 mmol) in THF (2.1 ml) was added to a stirred solution of (R)-4-(5-hydroxypyrimidin-2-yl)-3-methylpiperazine-1-carbonitrile (2.4 g, 10.95 mmol, INT 86) and (Z)—N′-hydroxyisobutyrimidamide (2.460 g, 24.08 mmol) in THF (30 ml) at 0° C., under nitrogen. The resulting solution was stirred at ambient temperature for 2 days. The reaction mixture was evaporated to dryness then hydrochloric acid (4.38 mL, 8.76 mmol) was added in toluene (75 mL) at 20° C. under nitrogen. The resulting solution was stirred at 80° C. for 16 hours, causing the reaction to turn black. The reaction mixture was concentrated and diluted with water (15 mL). The reaction mixture was neutralised with saturated Na₂CO₃, diluted with EtOAc (100 mL), and washed sequentially with water (50 mL) and saturated brine (50 mL). The organic layer was dried over MgSO₄, filtered and evaporated to afford crude product which was purified by flash silica chromatography, elution gradient 0 to 5% MeOH in DCM. Pure fractions were evaporated to dryness to afford (R)-2-(4-(3-isopropyl-1,2,4-oxadiazol-5-yl)-2-methylpiperazin-1-yl)pyrimidin-5-ol (0.800 g, 24%) as a orange oil.

INT 70

(R)-2-(2-methyl-4-(3-(trifluoromethyl)-1,2,4-oxadiazol-5-yl)piperazin-1-yl)pyrimidin-5-ol

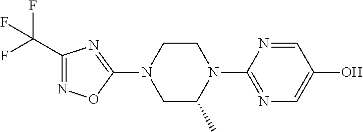

Zinc chloride (22.48 ml, 22.48 mmol) was added to (Z)-2,2,2-trifluoro-N′-hydroxyacetimidamide (1.766 g, 13.79 mmol) and (R)-4-(5-hydroxypyrimidin-2-yl)-3-methylpiperazine-1-carbonitrile (2.24 g, 10.22 mmol, INT 86) in THF (45 ml) and ethyl acetate (50 ml) over a period of 10 minutes under nitrogen. The resulting solution was stirred at 20° C. for 24 hours. All volatiles were removed under reduced pressure and the sticky solid obtained was triturated with Et₂O (50 ml) and the solid collected by filtration, washed with Et₂O (2×10 ml) and dried under suction. The solid was rinsed into a flask is with a mixture of EtOH and DCM. A sonic bath was required to dissolve some of the material. All volatiles were removed under reduced pressure to leave a pale brown sticky solid. Concentrated hydrochloric acid (10 ml, 0.00 μmol) in ethanol (100 ml) was added to this solid. The resulting solution was stirred at 110° C. (oil bath temperature) for 18 hours. All volatiles were removed under reduced pressure and the residue azeotroped with toluene (50 ml). The crude product was purified by suction flash silica chromatography by preabsorbing the material onto celite using mixtures of MeOH and DCM and columned by hand using an eluent of DCM to 100% EtOAc then MeOH:EtOAc (1:9). All product—containing fractions were combined and evaporated to dryness to afford (R)-2-(2-methyl-4-(3-(trifluoromethyl)-1,2,4-oxadiazol-5-yl)piperazin-1-yl)pyrimidin-5-ol (2.63 g, 58%) as a dark gum. ¹H NMR (400.13 MHz, CDCl₃) 1.18 (3H, d), 3.19-3.31 (2H, m), 3.44 (1H, dd), 3.93 (1H, dt), 4.08-4.15 (1H, m), 4.90 (1H, dt), 8.04 (2H, s). m/z (ES+) (M+H)+=331.31; HPLC tR=2.15 min.

INT 71

### 3-((R)-4-(5-((3-bromopyridin-4-yl)methoxy)pyrimidin-2-yl)-3-methylpiperazin-1-yl)-5-((S)-1-methoxyethyl)-1,2,4-oxadiazole

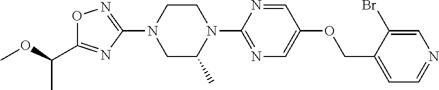

N-Ethyldiisopropylamine (0.508 ml, 2.94 mmol) was added to (R,E)-4-(5-((3-bromopyridin-4-yl)methoxy)pyrimidin-2-yl)-N′-hydroxy-3-methylpiperazine-1-carboximidamide (0.477 g, 1.13 mmol, INT 50), 1-hydroxybenzotriazole hydrate (0.259 g, 1.69 mmol), (S)-2-methoxypropanoic acid (0.165 g, 1.58 mmol) and N-(3-dimethylaminopropyl)-N′-ethylcarbodiimide hydrochloride (0.607 g, 3.16 mmol) in DMF (5 ml) and the resulting suspension stirred at 20° C. for 18 hours. Further portions of 1-hydroxybenzotriazole hydrate (0.259 g, 1.69 mmol), (S)-2-methoxypropanoic acid (0.165 g, 1.58 mmol) and N-(3-dimethylaminopropyl)-N′-ethylcarbodiimide hydrochloride (0.607 g, 3.16 mmol) was added followed by N-ethyldiisopropylamine (0.508 ml, 2.94 mmol) and the reaction stirred for another 18 hours. The reaction mixture was diluted with DCM (50 mL) and washed 2M K₂CO₃ aq. (20 ml). The organic layer was dried over Na₂SO₄, filtered and evaporated then suspended in toluene (25 mL) and stirred at 120° C. for 30 minutes. It was cooled to room temperature and concentrated in vacuo then purified by flash silica chromatography (40 g column), elution gradient 0 to 100% EtOAc in isohexane (material applied in DCM). The pure faster running spot fractions were combined and evaporated to dryness to afford 3-((R)-4-(5-((3-bromopyridin-4-yl)methoxy)pyrimidin-2-yl)-3-methylpiperazin-1-yl)-5-((S)-1-methoxyethyl)-1,2,4-oxadiazole (0.278 g, 50%), ¹H NMR (400 MHz, CDCl₃, 30° C.) d 1.25 (3H, d), 1.58 (3H, d), 3.08 (1H, td), 3.21-3.35 (2H, m), 3.43 (3H, s), 3.89 (1H, dt), 4.00-4.06 (1H, m), 4.43-4.54 (2H, m), 4.87-4.95 (1H, m), 5.07 (2H, s), 7.51 (1H, dd), 8.17 (2H, s), 8.56 (1H, d), 8.72 (1H, s). m/z (ES+) (M+H)+=492.

The following Intermediate was prepared in a similar manner to Intermediate 71, using the Intermediate 50 and (R)-2-methoxypropanoic acid:

| Structure and INT | Name | 1H NMR δ | MS |
|---|---|---|---|
| 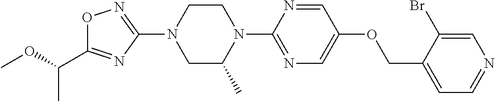 INT 72 | 3-((R)-4-(5-((3- bromopyridin-4- yl)methoxy) pyrimidin-2-yl)- 3-methylpiperazin- 1-yl)-5-((R)-1- methoxyethyl)- 1,2,4-oxadiazole | 1H NMR (400 MHz, CDCl3, 30° C.) d 1.25 (3H, d), 1.57 (3H, d), 3.00-3.14 (1H, m), 3.21-3.30 (1H, m), 3.30-3.38 (1H, m), 3.43 (3H, s), 3.88 (1H, dt), 3.98-4.08 (1H, m), 4.43-4.58 (2H, m), 4.91 (1H, ddd), 5.07 (2H, s), 7.48-7.57 (1H, m), 8.17 (2H, s), 8.56 (1H, d), 8.72 (1H, s). | m/z (ES+) (M + H)+ = 492 |

INT 73

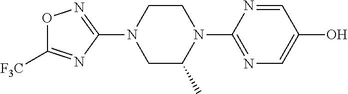

Hydroxylamine hydrochloride (3.48 g, 50.08 mmol) was added to (R)-4-(5-hydroxypyrimidin-2-yl)-3-methylpiperazine-1-carbonitrile (5.49 g, 25.04 mmol, INT 86) and sodium carbonate (3.11 g, 25.04 mmol) in DMF (80 mL) at 20° C. The resulting suspension was stirred at 80° C. for 30 minutes. The reaction was cooled to 25° C. and toluene (120 mL) was added, followed by pyridine (8.10 mL, 100.16 mmol) and trifluoroacetic anhydride (14.00 mL, 100.16 mmol) with water bath cooling. The reaction was stirred at 45° C. for 40 minutes then cooled, and the toluene evaporated. Ethyl acetate was added. The organic layer was separated, washed with water, then with brine, dried over Na₂SO₄, filtered and evaporated to give crude product that was purified by flash silica chromatography, elution gradient 10 to 40% EtOAc in DCM. Pure fractions were evaporated to dryness to afford (R)-2-(2-methyl-4-(5-(trifluoromethyl)-1,2,4-oxadiazol-3-yl)piperazin-1-yl)pyrimidin-5-ol (3.14 g, 38%) as a pale yellow oil. ¹H NMR (400 MHz, CDCl₃) 1.29 (3H, d), 3.15-3.23 (1H, m), 3.34-3.46 (2H, m), 3.88-3.94 (1H, m), 4.04-4.10 (1H, m), 4.40-4.48 (1H, m), 4.85-4.93 (1H, m), 8.27 (2H, s), 8.68 (1H, s). m/z (ES+) (M+H)+=331.

INT 74

### (R)-2-(2-methyl-4-(5-(3-methyloxetan-3-yl)-1,2,4-oxadiazol-3-yl)piperazin-1-yl)pyrimidin-5-ol

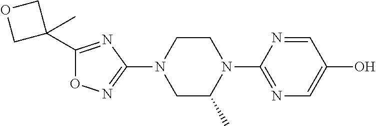

Sodium carbonate (0.426 g, 3.43 mmol) was added to (R)-4-(5-hydroxypyrimidin-2-yl)-3-methylpiperazine-1-carbonitrile (0.753 g, 3.43 mmol, INT 86) and hydroxylamine hydrochloride (0.477 g, 6.87 mmol) in DMF (20 mL) under nitrogen. The resulting solution was stiffed at 80° C. for 2 hours. N-Ethyldiisopropylamine (1.188 mL, 6.87 mmol), 1-Hydroxybenzotriazole (0.928 g, 6.87 mmol) and 3-methyloxetane-3-carboxylic acid (0.797 g, 6.87 mmol) were added in DMF (20 mL) under nitrogen. N-(3-Dimethylaminopropyl)-N′-ethylcarbodiimide hydrochloride (1.316 g, 6.87 mmol) was added and the resulting solution stirred at 20° C. for 18 hours. It was diluted with ethyl acetate (200 mL) and washed with water (4×80 mL) and brine (80 mL), dried (magnesium sulphate) and concentrated in vacuo. The product was dissolved in DCM (20 mL) and the suspension was filtered to yield (R)-2-(2-methyl-4-(5-(3-methyloxetan-3-yl)-1,2,4-oxadiazol-3-yl)piperazin-1-yl)pyrimidin-5-ol as a yellow solid which was used without further purification. ¹H NMR (400 MHz, CDCl₃, 30° C.) d 1.24 (3H, d), 1.81 (3H, s), 3.09 (1H, td), 3.20-3.43 (2H, m), 3.87 (1H, dt), 4.03 (1H, ddd), 4.36-4.50 (1H, m), 4.59 (2H, d), 4.87 (1H, ddd), 5.09 (2H, dd), 5.34 (1H, s), 8.17 (2H, s). m/z (ES+) (M+H)+=333

INT 75

### (R)-tert-butyl 4-(5-(2-fluoro-4-(methylsulfonylmethyl)benzyloxy)pyrimidin-2-yl)-3-methylpiperazine-1-carboxylate

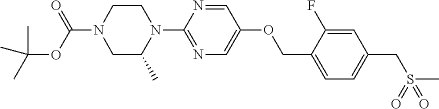

A solution of methanesulfonyl chloride (0.077 mL, 0.99 mmol) in DCM (1 mL) was added dropwise to a stirred solution of (R)-tert-butyl 4-(5-(2-fluoro-4-(hydroxymethyl)-benzyloxy)pyrimidin-2-yl)-3-methylpiperazine-1-carboxylate (0.306 g, 0.71 mmol, INT 76) and N-ethyldiisopropylamine (0.208 mL, 1.20 mmol) in DCM (8 mL) cooled to 0° C., over a period of 5 minutes under nitrogen. The resulting solution was stirred at 0° C. for 1 hour. The reaction mixture was diluted with DCM (50 mL), and washed with 1M citric acid (15 mL). The organic layer was dried over MgSO₄, filtered and evaporated to afford crude product. To this was added lithium iodide (0.190 g, 1.42 mmol) and dioxane (3 mL) and the reaction was heated at 60° C. for 1 hour and then at room temperature overnight. The reaction mixture was diluted with EtOAc (50 mL) and washed with a mixture of saturated ammonium chloride aq and 10% aq. sodium thiosulphate (20 ml). The organic layer was dried over MgSO₄, filtered to give a yellow gum. Sodium methanesulfinate (0.087 g, 0.85 mmol) was added to the gum in DMF (5 mL) at 22° C. under air. The resulting mixture was stirred at 22° C. for 1 hour. The reaction mixture was diluted with EtOAc (50 mL), and washed sequentially with 10% aq. sodium thiosulphate (20 mL) and saturated brine (15 mL). The organic layer was dried over MgSO₄, filtered and evaporated to afford crude product. The crude product was purified by flash silica chromatography (40 g column), elution gradient 0 to 100% EtOAc in isohexane (material applied in DCM). Pure fractions were evaporated to dryness to afford (R)-tert-butyl 4-(5-(2-fluoro-4-(methylsulfonylmethyl)benzyloxy)pyrimidin-2-yl)-3-methylpiperazine-1-carboxylate (0.292 g, 83%) as a white solid. ¹H NMR (400 MHz, CDCl₃, 30° C.) 1.15 (3H, d), 1.49 (9H, s), 2.78-3.20 (6H, m), 3.78-4.38 (5H, m), 4.68-4.84 (1H, m), 5.09 (2H, s), 7.17-7.29 (3H, m), 7.54 (1H, t), 8.14 (2H, s). m/z (ES+) (M+H)+=495

INT 76

### (R)-tert-butyl 4-(5-(2-fluoro-4-(hydroxymethyl)benzyloxy)pyrimidin-2-yl)-3-methylpiperazine-1-carboxylate

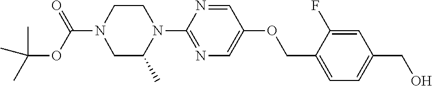

To a stirred solution of (R)-tert-butyl 4-(5-(2-fluoro-4-formylbenzyloxy)pyrimidin-2-yl)-3-methylpiperazine-1-carboxylate (3.65 g, 8.48 mmol) in ethanol (30 ml) at 0° C. was added sodium borohydride (0.449 g, 11.87 mmol) in one portion. The suspension gradually became a clear solution. When the addition was completed, the mixture was allowed to come to ambient temperature and stirred for 3 hours. Saturated NH₄Cl aq (10 ml) was added cautiously and the mixture was diluted with EtOAc (150 mL), and washed sequentially with water (10 mL) and saturated brine (10 mL). The organic layer was dried over Na₂SO₄, filtered and evaporated to afford (R)-tert-butyl 4-(5-(2-fluoro-4-(hydroxymethyl)benzyloxy)pyrimidin-2-yl)-3-methylpiperazine-1-carboxylate (3.34 g, 91%) as a white solid. ¹H NMR (400 MHz, DMSO, 100° C.) 1.10 (3H, d), 1.46 (9H, s), 2.89-3.00 (1H, m), 3.06-3.19 (2H, m), 3.82 (1H, dt), 3.91-3.99 (1H, m), 4.23-4.30 (1H, m), 4.55 (2H, d), 4.68-4.76 (1H, m), 4.99 (1H, t), 5.13 (2H, s), 7.13-7.21 (2H, m), 7.47 (1H, t), 8.24 (2H, s). m/z (ES+) (M+H)+=433.

INT 77

### (R)-tert-butyl 4-(5-(2-fluoro-4-formylbenzyloxy)pyrimidin-2-yl)-3-methylpiperazine-1-carboxylate

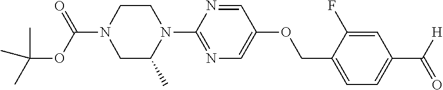

Butyllithium (1.6M solution in hexanes) (8.52 ml, 13.63 mmol) was added dropwise to (R)-tert-butyl 4-(5-(4-bromo-2-fluorobenzyloxy)pyrimidin-2-yl)-3-methylpiperazine-1-carboxylate (5.25 g, 10.91 mmol, INT 29) in anhydrous THF (40 ml) cooled to −90° C. (Et₂O/liquid N₂) under nitrogen. The resulting solution was stirred at −90° C. for 10 minutes. To this solution was then added dropwise N,N-dimethylformamide (1.942 ml, 25.09 mmol) at −90° C. under nitrogen. The resulting mixture was stirred at −78° C. for 1 hour then allowed to slowly warm to room temperature. The reaction mixture was quenched with saturated aq. NH₄Cl (30 ml), extracted with EtOAc (2×300 mL). The organic layer was dried over MgSO₄, filtered and evaporated to afford the crude product as an orange oil which crystallised on standing. The crude product was purified by flash silica chromatography, elution gradient 0 to 100% EtOAc in isohexane to afford (R)-tert-butyl 4-(5-(2-fluoro-4-formylbenzyloxy)pyrimidin-2-yl)-3-methylpiperazine-1-carboxylate (3.92 g, 83%), as a yellow solid. ¹H NMR (400 MHz, DMSO, 100° C.) d 1.08 (3H, d), 1.44 (9H, s), 2.87-2.96 (1H, m), 3.05-3.15 (2H, m), 3.79 (1H, dt), 3.89-3.96 (1H, m), 4.20-4.30 (1H, m), 4.66-4.75 (1H, m), 5.23 (2H, s), 7.69 (1H, d), 7.74-7.82 (2H, m), 8.26 (2H, s), 10.02 (1H, d). m/z (ES+) (M+H)+=431

INT 78

### Perfluorophenyl 3-(trifluoromethyl)oxetan-3-yl carbonate

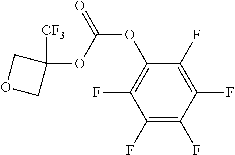

Tetrabutylammonium fluoride (1M in THF) (2.86 mL, 2.86 mmol) was added to oxetan-3-one (2.06 g, 28.59 mmol) and trimethyl(trifluoromethyl)silane (2M in THF) (23.58 mL, 47.17 mmol, 1.65 eq.) in THF (25 mL) at 20° C. under nitrogen. An ice bath was used to control the exotherm. The resulting dark brown solution was stiffed at 20° C. for 2 hours. 6M hydrochloric acid (60 mL) was added at 0° C. then the temperature was allowed to rise to and stirred at 20° C. for 2 hours. The reaction mixture was diluted with Et₂O (100 mL), and washed with saturated brine (50 mL). The aqueous was extracted with DCM (2×100 ml). The combined organic layers were dried over Na₂SO₄ and filtered. The filtrate was gently evaporated (stopped at ˜400 mbar) to remove solvent then to the mixture was added bis(perfluorophenyl)carbonate (13.52 g, 34.31 mmol) and acetonitrile (15 mL). To this mixture was added triethylamine (12.75 mL, 91.49 mmol) dropwise at 0° C. over a period of 5 minutes under nitrogen. The resulting solution was allowed to warm to room temperature then stirred at 20° C. for 18 hours. All volatiles were removed under reduced pressure to leave a purple oil. The crude product was purified by flash silica chromatography, elution gradient 0 to 100% DCM in isohexane. Pure fractions were evaporated to dryness to afford perfluorophenyl 3-(trifluoromethyl)oxetan-3-yl carbonate (8.38 g, 83%) as a yellow oil.

¹H NMR (400 MHz, CDCl₃, 30° C.) 4.88-4.93 (2H, m), 5.02-5.07 (2H, m).

INT 79

### (R)-5-(2-fluoro-4-(methylsulfonyl)benzyloxy)-2-(2-methylpiperazin-1-yl)pyrimidine

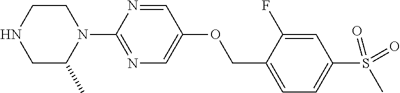

Hydrogen chloride (4M in dioxane) (57.7 mL, 230.98 mmol) was added to a stirred solution of (R)-tert-butyl 4-(5-(2-fluoro-4-(methylsulfonyl)benzyloxy)pyrimidin-2-yl)-3-methylpiperazine-1-carboxylate (11.1 g, 23.10 mmol, Example 35) in dichloromethane (100 mL) and the mixture stirred at ambient temperature for 16 hours then evaporated in vacuo to give a foamy solid which was triturated with diethyl ether and filtered to give (R)-5-(2-fluoro-4-(methylsulfonyl)benzyloxy)-2-(2-methylpiperazin-1-yl)pyrimidine (7.35 g, 76%) as a pale yellow solid which was hygroscopic. m/z (ES+) (M+H)+=381.

INT 80

### (R)-tert-butyl 4-cyano-2-methylpiperazine-1-carboxylate

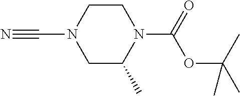

To a stirred solution of (R)-tert-butyl 2-methylpiperazine-1-carboxylate (1.000 g, 4.99 mmol) in dichloromethane (21.64 ml) was added sodium hydrogen carbonate (2.097 g, 24.97 mmol) in water (6.66 ml) at 0° C. To this stirred mixture was added a solution of cyanogen bromide (0.635 g, 5.99 mmol) in dichloromethane (21.64 ml) at 0° C. The mixture was stirred at 0° C. for 30 minutes, then allowed to come to ambient temperature and stirred for 2 hours. The layers were separated and the dichloromethane extract washed with saturated sodium hydrogen carbonate solution, dried (MgSO₄) and evaporated in vacuo to give (R)-tert-butyl 4-cyano-2-methylpiperazine-1-carboxylate (1.120 g, 100%). ¹H NMR (400 MHz, CDCl₃) 1.2 (3H, d), 1.4 (9H, s), 2.95-3.3 (5H, m), 3.8 (1H, d), 4.25 (1H, br)

INT 81

### (R)-tert-butyl 4-(5-isopropyl-1,2,4-oxadiazol-3-yl)-2-methylpiperazine-1-carboxylate

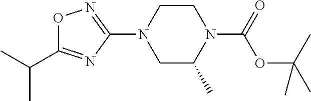

To a stirred solution of (R)-tert-butyl 4-cyano-2-methylpiperazine-1-carboxylate (1.100 g, 4.88 mmol) in DMF (19.5 ml) was added hydroxylamine hydrochloride (0.679 g, 9.77 mmol) and sodium carbonate (0.518 g, 4.88 mmol). The stirred mixture was heated at 80° C. for 1 hour and cooled to ambient temperature to give (R)-tert-butyl 4-(N-hydroxycarbamimidoyl)-2-methylpiperazine-1-carboxylate (1.260 g, 100%) which was used without further purification in the next step. The compound was dissolved in DMF (19.5 mL) and then treated with isobutyric acid (0.905 mL, 9.76 mmol), N,N-diisopropylethylamine (1.837 mL, 10.73 mmol), 1-Hydroxybenzotriazole (1.450 g, 10.73 mmol) and N-(3-dimethylaminopropyl)-N′-ethylcarbodiimide hydrochloride (2.244 g, 11.71 mmol) and the mixture was stirred at ambient temperature for 16 hours. The mixture was poured onto water (200 mL), extracted with ethyl acetate (3×75 mL), and the combined ethyl acetate extracts washed with brine, dried (MgSO4) and evaporated in vacuo to give a solid that was suspended in toluene (43 mL) and the stirred mixture was heated under reflux at 120° C. for 1 hour. The mixture was cooled to ambient temperature and the toluene evaporated in vacuo to a residue which was chromatographed on silica with 20% ethyl acetate in isohexane as eluant to give (R)-tert-butyl 4-(5-isopropyl-1,2,4-oxadiazol-3-yl)-2-methylpiperazine-1-carboxylate (0.980 g, 65%). ¹H NMR (400 MHz, CDCl₃) 1.15 (3H, d), 1.3 (6H, d), 1.4 (9H, s), 2.8-3.15 (4H, m), 3.65 (1H, d), 3.8-3.9 (2H, m), 4.2-4.3 (1H, m).

INT 82

### (R)-5-isopropyl-3-(3-methylpiperazin-1-yl)-1,2,4-oxadiazole

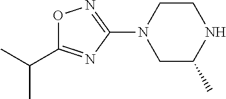

To a stirred solution of (R)-tert-butyl 4-(5-isopropyl-1,2,4-oxadiazol-3-yl)-2-methyl-piperazine-1-carboxylate (189 mg, 0.61 mmol) in dichloromethane (4.575 mL) was added 4M hydrochloric acid in dioxane (1.525 mL, 6.10 mmol) and the mixture stirred at ambient temperature for 2 hours. The dichloromethane and dioxane were evaporated in vacuo to give (R)-5-isopropyl-3-(3-methylpiperazin-1-yl)-1,2,4-oxadiazole (172 mg, 100%) which was dried under high vacuum and used without further purification. ¹H NMR (400 MHz, CDCl₃) 1.0 (3H, d), 1.3 (6H, d), 2.5 (1H, t), 2.8-3.0 (5H, m), 3.3-3.4 (2H, m). m/z (ES+) (M+H)+=211.

INT 83

### 5-((3-bromopyridin-4-yl)methoxy)-2-chloropyrimidine

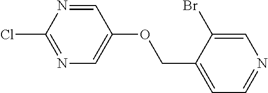

To a stirred solution of (3-bromopyridin-4-yl)methanol (2.377 g, 12.64 mmol) and triethylamine (3.52 mL, 25.28 mmol) in dichloromethane (52.6 mL) was added a solution of methanesulfonyl chloride (1.034 mL, 13.27 mmol) in dichloromethane (5.26 mL) at 5° C.-10° C. When the addition was completed the mixture was stiffed at 5° C.-10° C. for 2 hours, treated with acetonitrile (52.6 mL), and then the dichloromethane evaporated in vacuo to leave a solution of the mesylate in acetonitrile, which was treated with 2-chloropyrimidin-5-ol (1.500 g, 11.49 mmol) and potassium carbonate (4.76 g, 34.47 mmol) and the stirred mixture heated under reflux at 85° C. overnight. The mixture was cooled to ambient temperature and the acetonitrile evaporated in vacuo to a residue which was partitioned between water (50 mL) and ethyl acetate (100 mL), and filtered through a filtration aid. The ethyl acetate layer washed with brine, dried (MgSO₄) and evaporated in vacuo to a residue which was chromatographed on silica with 50% ethyl acetate in isohexane as eluant to give a solid which was crystallised from ethyl acetate/isohexane to give 5-((3-bromopyridin-4-yl)methoxy)-2-chloropyrimidine (2.370 g, 69%). ¹H NMR (400.13 MHz, CDCl₃) 5.1 (2H, s), 7.4 (1H, d), 8.3 (2H, s), 8.5 (1H, d), 8.7 (1H, s). m/z (ES+) (M+MeCN)+=343.

INT 84

### 4-((2-chloropyrimidin-5-yloxy)methyl)nicotinonitrile

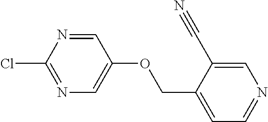

To a mixture of 5-((3-bromopyridin-4-yl)methoxy)-2-chloropyrimidine (273 mg, 0.91 mmol), zinc cyanide (64.0 mg, 0.55 mmol), tris(dibenzylideneacetone)dipalladium(0) (33.3 mg, 0.04 mmol) and 4,5-bis(diphenylphosphino)-9,9-dimethylxanthene (42.0 mg, 0.07 mmol) in a microwave vial under an atmosphere of nitrogen was added degassed DMF (5.0 mL). The stirred mixture was heated at 120° in a Biotage Initiator Microwave oven for 2 hours, cooled to ambient temperature, and the mixture poured onto water (75 mL) and ethyl acetate (75 mL), and filtered through a filtration aid. The aqueous layer was extracted with ethyl acetate (2×75 mL) and the combined ethyl acetate extracts were washed with brine, dried (MgSO₄) and evaporated in vacuo to a residue which was chromatographed on silica with 50% ethyl acetate in isohexane as eluant to give 4-((2-chloropyrimidin-5-yloxy)methyl)nicotinonitrile (160 mg, 71%). ¹H NMR (400.13 MHz, CDCl3) 5.25 (2H, s), 7.55 (1H, d), 8.35 (2H, s), 8.85 (1H, d), 8.9 (1H, s). m/z (ES−) (M−H)−=245.

INT 85

### (R)-2-(2-methylpiperazin-1-yl)pyrimidin-5-ol dihydrochloride

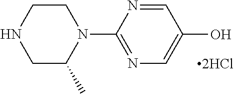

Hydrogen chloride (4M in dioxane) (34.0 mL, 135.89 mmol) was added to (R)-tert-butyl 4-(5-hydroxypyrimidin-2-yl)-3-methylpiperazine-1-carboxylate (4.0 g, 13.59 mmol, INT 10) in DCM (40 mL) at 20° C. The resulting solution was stirred at 20° C. for 90 minutes. The reaction was evaporated. The crude solid obtained was triturated with Et₂O to give a solid which was collected by filtration and dried under vacuum to give (R)-2-(2-methylpiperazin-1-yl)pyrimidin-5-ol dihydrochloride (3.49 g, >100%) as a white solid. ¹H NMR (400 MHz, DMSO) 1.21 (3H, d), 2.82-2.95 (1H, m), 3.05-3.31 (4H, m), 4.37-4.42 (1H, m), 4.81-4.85 (1H, m), 8.09 (2H, s), 9.06 (1H, s), 9.56 (1H, s). m/z (ES+) (M+H)+=195.28; HPLC tR=0.64 min.

INT 86

### (R)-4-(5-hydroxypyrimidin-2-yl)-3-methylpiperazine-1-carbonitrile

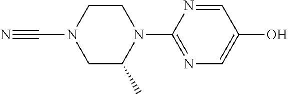

A slurry of sodium bicarbonate (4.39 g, 52.25 mmol) in water (20 mL) was added to (R)-2-(2-methylpiperazin-1-yl)pyrimidin-5-ol dihydrochloride (3.49 g, 13.06 mmol) in DCM (70 mL) at 0° C. A solution of cyanogen bromide (1.660 g, 15.68 mmol) in DCM (10 mL) was added and the resulting suspension stiffed at 0° C. for 30 minutes and rt for 30 minutes. The mixture was washed with saturated aqueous sodium bicarbonate (50 mL) and the aqueous acidified and extracted into EtOAc and the combined organics dried over Na₂SO₄, filtered and evaporated. The crude product was purified by flash silica chromatography, elution gradient 0 to 5% MeOH in DCM. Pure fractions were evaporated to dryness to afford (R)-4-(5-hydroxypyrimidin-2-yl)-3-methylpiperazine-1-carbonitrile (1.700 g, 59%) as a white solid. ¹H NMR (400 MHz, CDCl₃) 1.30 (3H, d), 3.13-3.27 (3H, m), 3.28-3.37 (1H, m), 3.40-3.46 (1H, m), 4.40-4.45 (1H, m), 4.85-4.90 (2H, m), 8.08 (2H, s). m/z (ES+) (M+H)+=220.34; HPLC tR=0.65 min.

INT 87

### (R)-2-(4-(5-(difluoromethyl)-1,2,4-oxadiazol-3-yl)-2-methylpiperazin-1-yl)pyrimidin-5-ol

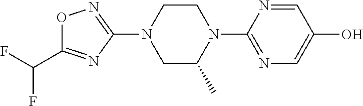

Hydroxylamine hydrochloride (0.919 g, 13.23 mmol) was added to (R)-4-(5-hydroxypyrimidin-2-yl)-3-methylpiperazine-1-carbonitrile (1.45 g, 6.61 mmol, INT 86) and sodium carbonate (0.820 g, 6.61 mmol) in DMF (12 mL) at 20° C. The resulting suspension was stirred at 80° C. for 30 minutes. Toluene (18.00 mL) was added, followed by pyridine (2.140 mL, 26.45 mmol) and difluoroacetic anhydride (3.29 mL, 26.45 mmol). The reaction was stirred at 80° C. for 1 hour. The reaction was cooled and the toluene evaporated. Ethyl acetate was added and then the mixture was washed with water and brine, dried over Na₂SO₄, filtered and evaporated to give crude product which was purified by flash silica chromatography, elution gradient 10 to 30% EtOAc in DCM. Pure fractions were evaporated to dryness to afford (R)-2-(4-(5-(difluoromethyl)-1,2,4-oxadiazol-3-yl)-2-methylpiperazin-1-yl)pyrimidin-5-ol (1.800 g, 87%) as a pale yellow oil which solidified on standing. ¹H NMR (400 MHz, CDCl₃) 1.23 (3H, d), 3.09-3.18 (1H, m), 3.29-3.36 (2H, m), 3.69 (1H, s), 3.86-3.90 (1H, m), 4.00-4.05 (1H, m), 4.40-4.47 (1H, m), 4.85-4.92 (1H, m), 6.64 (1H, t), 8.13 (2H, s). m/z (ES+) (M+H)+=313.26; HPLC tR=1.37 min.

1) A compound of formula I 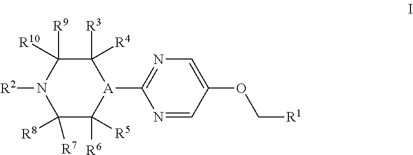
or a pharmaceutically acceptable salt thereof in which
A represents N or CH;
R¹ represents a) a phenyl ring substituted in the 4-position by one of the groups 1 to 6 below and wherein the phenyl ring is optionally additionally substituted in the 2 and/or the 3 and/or the 5 and/or the 6 position by a group independently selected one or more of the following: cyano, fluoro, hydroxy, a C₃₋₄cycloalkoxy, a C₁₋₄alkoxy optionally substituted by one or more fluoro or a C₁₋₄alkyl optionally substituted by hydroxy or C₁₋₄alkoxyor by one or more fluoro;
1) a group —N(R¹¹)COR¹² in which R¹¹ represents H or a C₁₋₆alkyl optionally substituted by one or more of the following: fluoro, hydroxy or C₁₋₄alkoxy and R¹² represents a C₁₋₆alkyl optionally substituted by one or more of the following: fluoro, hydroxy, a C₃₋₆cycloalkyl, C₁₋₄alkoxy or a group —NR¹³R¹⁴ in which R¹³ and R¹⁴ independently represent H, a C₁₋₆alkyl optionally substituted by one or more of the following: fluoro, hydroxy or C₁₋₄alkoxy or R¹² represents a C₃₋₆cycloalkyl optionally substituted by one or more of the following: fluoro, hydroxy, C₁₋₄alkyl or C₁₋₄alkoxy or R¹² represents a group (CH₂)ₖ-Het wherein k is 0, 1, 2, 3 or 4 and Het represents a carbon linked saturated 4 to 7 membered heterocyclic group containing one or more N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
2) a group —CONR¹⁵R¹⁶ in which R¹⁵ and R¹⁶ independently represent H, C₃₋₆cycloalkyl or a C₁₋₆alkyl optionally substituted by one or more of the following i) fluoro ii) hydroxy iii) C₁₋₄alkoxy
iv) C₃₋₆cycloalkyl or v) a group —NR¹⁷R¹⁸ in which R¹⁷ and R¹⁸ independently represent H or a C₁₋₆alkyl optionally substituted by one or more of the following: fluoro, hydroxy, C₁₋₄alkylor C₁₋₄alkoxy; or R¹⁷ and R¹⁸ together with the nitrogen to which they are attached represent a saturated 4-7 membered heterocyclic group optionally containing an additional N, S or O and wherein the heterocyclic group is optionally substituted by one or more of the following: fluoro, hydroxy, oxo, C₁₋₄alkyl or C₁₋₄alkoxy; v) a carbon linked saturated 4 to 7 membered heterocyclic group containing one or more N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy; or R¹⁵ and R¹⁶ together with the nitrogen to which they are attached represent a saturated 4-7 membered heterocyclic group optionally containing an additional N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
is 3) a group —(CH₂)₁—(O)ₘS(O)ₙR¹⁹ in which m is 0 or 1 and when m is 0 then 1 is 0, 1, 2, 3, or 4 and n is 1 or 2 and when m is 1 then 1 is 0 and n is 2 and R¹⁹ represents a C₁₋₆alkyl optionally substituted by one or more of the following: fluoro, hydroxy, C₃₋₆cycloalkyl, C₁₋₄alkyl or C₁₋₄alkoxy; or by a group —NR²⁰R²¹ in which R²⁰ and R²¹ independently represent H, C₃₋₆cycloalkyl or a C₁₋₆alkyl or R²⁰ and R²¹ together with the nitrogen to which they are attached represent a saturated 4-7 membered heterocyclic group optionally containing an additional N, S or O wherein the S may be in its oxidised form of SO or SO₂ and wherein the heterocyclic group is optionally substituted by one or more of the following: fluoro, hydroxy, oxo, C₁₋₄alkyl or C₁₋₄alkoxy;
or R¹⁹ represents C₃₋₆cycloalkyl optionally substituted by one or more of the following: fluoro, hydroxy, C₁₋₄alkyl or C₁₋₄alkoxy;
or R¹⁹ represents a carbon linked saturated 4 to 7 membered heterocyclic group containing one or more N, S or O wherein the S may be in its oxidised form of SO or SO₂ , and wherein the heterocyclic group is optionally substituted by one or more of the following: fluoro, hydroxy, oxo, C₁₋₄alkyl or C₁₋₄alkoxy;
4) a group —N(R²²)CON(R²³)(R²⁴) in which R²², R²³ and R²⁴ independently represent H or a C₁₋₆alkyl group;
5) a group SO₂NR²⁵R²⁶ in which R²⁵ and R²⁶ independently represent H, a C₁₋₆alkyl group or a C₃₋₆cycloalkyl group wherein the alkyl and cycloalkyl groups are optionally substituted by one or more of the following: fluoro, hydroxy, C₁₋₄alkyl or C₁₋₄alkoxy;
or R²⁵ and R²⁶ together with the nitrogen to which they are attached represent a saturated 4-7 membered heterocyclic group optionally containing an additional N, S or O and wherein the heterocyclic group is optionally substituted by one or more of the following: fluoro, hydroxy, oxo, C₁₋₄alkyl or C₁₋₄alkoxy;
6) a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy;
or R¹ represents
b) 4-pyridyl optionally substituted by one or more of the following: halo, cyano, C₁₋₄alkyl, C₁₋₄alkoxy, C₁₋₄alkylsulfonyl or a group CONR²⁷R²⁸ in which R²⁷ and R²⁸ independently represent H or a C₁₋₆alkyl group; or
c) 2-pyridyl substituted in the 5-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the pyridyl ring is optionally additionally substituted by one or more of the following: halo, cyano, C₁₋₄alkyl or C₁₋₄alkoxy; or
d) 3-pyridyl substituted in the 6-position by C₁₋₄alkylsulfonyl, C₂₋₄alkanoylamino or by a 5-membered heteroaromatic group containing 1, 2, 3 or 4 hetero atoms selected from O, N and S optionally substituted by one or more of the following: fluoro, hydroxy, C₁₋₄alkyl or C₁₋₄alkoxy; and wherein the pyridyl ring is optionally additionally substituted by one or more of the following: halo, cyano, C₁₋₄alkyl or C₁₋₄alkoxy; or
e) pyrimidin-4-yl or pyrimidin-5-yl optionally substituted in the 2 position by a C₁₋₆alkanoylamino group or by cyano;
R² represents 1) a group —CO—OR^{x} in which R^{x} represents a C₁₋₆alkyl optionally substituted by cyano, hydroxy, C₁₋₄alkoxy or by one or more fluoro or R^{x} represents C₃₋₆cycloalkyl optionally substituted by one or more of the following: fluoro, hydroxy, C₁₋₄alkyl or C₁₋₄alkoxy; or R^{x} represents a saturated cyclic ether containing an oxygen and 3, 4 or 5 carbons optionally substituted by one or more of the following: fluoro, hydroxy, C₁₋₄alkyl optionally substituted by one or more fluoro or C₁₋₄alkoxy optionally substituted by one or more fluoro;
2) 2-pyrimidyl optionally substituted by one or more of the following: cyano, one or more halo, C₁₋₄alkoxy which is optionally substituted by one or more fluoro, C₃₋₆cycloalkyl optionally substituted by one or more of the following: fluoro, hydroxy, C₁₋₄alkyl or C₁₋₄alkoxy; or C₁₋₄alkyl which is optionally substituted by one or more of the following: fluoro, hydroxy, C₁₋₄alkyl or C₁₋₄alkoxy; or
3) 1,2,4-oxadiazol-3-yl or 1,2,4-oxadiazol-5-yl each of which is optionally substituted by cyano, by one or more halo, by C₁₋₄alkoxy which is optionally substituted by one or more fluoro, by C₃₋₆cycloalkyl optionally substituted by one or more of the following: fluoro, hydroxy, C₁₋₄alkyl or C₁₋₄alkoxy; or by C₁₋₄alkyl which is optionally substituted by one or more of the following: fluoro, hydroxy, C₁₋₄alkyl or C₁₋₄alkoxy;
R³, R⁴, R⁵, R⁶, R⁷, R⁸, R⁹ and R¹⁰ independently represent H or a C₁₋₄alkyl group optionally substituted by one or more of the following: fluoro, hydroxy, C₁₋₄alkyl or C₁₋₄alkoxy; or R³ and R⁷ together represent a methylene or ethylene bridge, or R⁷ and R⁹ together represent a methylene or ethylene bridge, or R³ and R⁵ together represent a methylene or ethylene bridge; and additionally when A is CH then R³ and R⁵ may also be independently selected from fluoro, hydroxy, or C₁₋₄alkoxy.

2) A compound as claimed in claim 1 of formula II 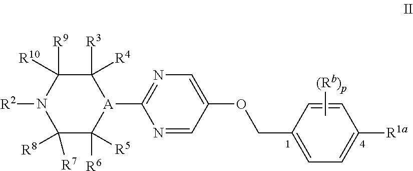
or a pharmaceutically acceptable salt thereof in which
A represents N or CH;
R^{1a} represents a group selected from one of groups 1-6 in R¹ above;
p=0 or 1 and R^{b} fluoro or C₁₋₃alkyl;
and R², R³, R⁴, R⁵, R⁶, R⁷, R⁸, R⁹ and R¹⁰ are as in claim 1.

3) A compound as claimed in claim 1 of formula III 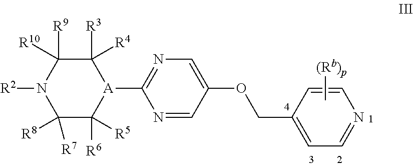
or a pharmaceutically acceptable salt thereof in which
A represents N or CH;
p=0, 1 or 2; R^{b} is bromo, fluoro, cyano, C₁₋₄alkoxy or C₁₋₄alkyl,
and R², R³, R⁴, R⁵, R⁶, R⁷, R⁸, R⁹ and R¹⁰ are as described in claim 1.

4) A compound as claimed in claim 1 of formula IV 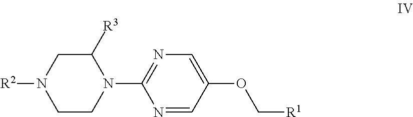
or a pharmaceutically acceptable salt thereof in which
R¹ represents phenyl substituted in the 4-position by a C₁₋₄alkylsulfonyl group or by a C₁₋₄alkylsulfonyloxy group or by a C₁₋₄alkylsulfonylC₁₋₄alkyl group and optionally the phenyl is additionally substituted by fluoro; or R¹ represents 4-pyridyl optionally substituted by cyano;
R² represents 1,2,4-oxadiazol-3-yl or 1,2,4-oxadiazol-5-yl each of which is optionally substituted by a C₁₋₄alkyl group which is optionally substituted by C₁₋₄alkoxy or by one or more fluoro or R² represents a group —COOR^{x} wherein R^{x} represents a C₁₋₄alkyl group optionally substituted by one or more fluoro or R^{x} represents oxetan-3-yl optionally substituted by a C₁₋₄alkyl group which is optionally substituted by one or more fluoro; and
R³ represents methyl.

5) A compound as claimed in claim 1 of formula IV 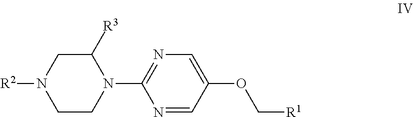
or a pharmaceutically acceptable salt thereof in which
R¹ represents 3-cyanopyridin-4-yl, 2-fluoro-4-(methylsulfonyl)phenyl or 2-fluoro-4-(methylsulfonylmethyl)phenyl;
R² represents 5-isopropyl-1,2,4-oxadiazol-3-yl, 3-isopropyl-1,2,4-oxadiazol-5-yl, 3-(trifluoromethyl)-1,2,4-oxadiazol-5-yl, 5-((S)-1-methoxyethyl)-1,2,4-oxadiazol-3-yl, 5-((R)-1-methoxyethyl)-1,2,4-oxadiazol-3-yl, or a group —COOR^{x} wherein R^{x} is (R)-1,1,1-trifluoropropan-2-yl), (S)-1,1,1-trifluoropropan-2-yl or (R)-3-(trifluoromethyl)oxetan-3-yl: and
R³ represents methyl.

6) A compound as claimed in any one of claims 1 to 3 in which A is N.

7) A compound as claimed in any one of claims 1 to 6.

8) A compound as claimed in claim 1 selected from one or more of the following:
tert-butyl 4-(5-(4-(methylsulfonyl)benzyloxy)pyrimidin-2-yl)piperazine-1-carboxylate;
tert-butyl 4-(5-(4-(1H-1,2,4-triazol-1-yl)benzyloxy)pyrimidin-2-yl)piperazine-1-carboxylate;
tert-butyl 4-(5-(pyridin-4-ylmethoxy)pyrimidin-2-yl)piperazine-1-carboxylate;
tert-butyl 4-(5-(4-(5-methyl-1,2,4-oxadiazol-3-yl)benzyloxy)pyrimidin-2-yl)piperazine-1-carboxylate;
tert-butyl 4-(5-((6-(1H-pyrazol-1-yl)pyridin-3-yl)methoxy)pyrimidin-2-yl)piperazine-1-carboxylate;
tert-butyl 4-(5-((6-acetamidopyridin-3-yl)methoxy)pyrimidin-2-yl)piperazine-1-carboxylate;
tert-butyl 4-(5-(4-(1H-tetrazol-1-yl)benzyloxy)pyrimidin-2-yl)piperazine-1-carboxylate;
tert-butyl 4-(5-((5-fluoro-2-methoxypyridin-4-yl)methoxy)pyrimidin-2-yl)piperazine-1-carboxylate;
tert-butyl 4-(5-(pyrimidin-4-ylmethoxy)pyrimidin-2-yl)piperazine-1-carboxylate;
tert-butyl 4-(5-((6-(1H-1,2,4-triazol-1-yOpyridin-3-yl)methoxy)pyrimidin-2-yl)piperazine-1-carboxylate;
tert-butyl 4-(5-((3-methoxypyridin-4-yl)methoxy)pyrimidin-2-yl)piperazine-1-carboxylate;
tert-butyl 4-(5-(4-isobutyramido-3-methylbenzyloxy)pyrimidin-2-yl)piperazine-1-carboxylate;
tert-butyl 4-(5-(3-methyl-4-pivalamidobenzyloxy)pyrimidin-2-yl)piperazine-1-carboxylate;
tert-butyl 4-(5-(4-isobutyramidobenzyloxy)pyrimidin-2-yl)piperazine-1-carboxylate;
tert-butyl 4-(5-(4-pivalamidobenzyloxy)pyrimidin-2-yl)piperazine-1-carboxylate;
tert-butyl 4-(5-(4-(N-methylsulfamoyl)benzyloxy)pyrimidin-2-yl)piperazine-1-carboxylate;
tert-butyl 4-(5-(4-(methylsulfonyloxy)benzyloxy)pyrimidin-2-yl)piperazine-1-carboxylate;
(R)-tert-butyl 3-methyl-4-(5-(4-(methylsulfonyl)benzyloxy)pyrimidin-2-yl)piperazine-1-carboxylate;
(R)-tert-butyl 3-methyl-4-(5-(pyridin-4-ylmethoxy)pyrimidin-2-yl)piperazine-1-carboxylate;
(R)-tert-butyl 4-(5-(4-(1H-tetrazol-1-yl)benzyloxy)pyrimidin-2-yl)-3-methylpiperazine-1-carboxylate;
(R)-tert-butyl 4-(5-(4-((2-hydroxyethyl)(methyl)carbamoyl)benzyloxy)pyrimidin-2-yl)-3-methylpiperazine-1-carboxylate;
(S)-tert-butyl 2-methyl-4-(5-(4-(methylsulfonyl)benzyloxy)pyrimidin-2-yl)piperazine-1-carboxylate;
(R)-tert-butyl 2-methyl-4-(5-(4-(methylsulfonyl)benzyloxy)pyrimidin-2-yl)piperazine-1-carboxylate;
(S)-tert-butyl 3-methyl-4-(5-(4-(methylsulfonyl)benzyloxy)pyrimidin-2-yl)piperazine-1-carboxylate;
(2R,5S)-tert-butyl 2,5-dimethyl-4-(5-(4-(methylsulfonyl)benzyloxy)pyrimidin-2-yl)piperazine-1-carboxylate;
(3R,5S)-tert-butyl 3,5-dimethyl-4-(5-(4-(methylsulfonyl)benzyloxy)pyrimidin-2-yl)piperazine-1-carboxylate;
tert-butyl 3,3-dimethyl-4-(5-(4-(methylsulfonyl)benzyloxy)pyrimidin-2-yl)piperazine-1-carboxylate;
(1R,4R)-tert-butyl 5-(5-(4-(methylsulfonyl)benzyloxy)pyrimidin-2-yl)-2,5-diazabicyclo[2.2.1]heptane-2-carboxylate;
(1S,4S)-tert-butyl 5-(5-(4-(methylsulfonyl)benzyloxy)pyrimidin-2-yl)-2,5-diazabicyclo[2.2.2]octane-2-carboxylate;
tert-butyl 4-(5-((6-(methylsulfonyl)pyridin-3-yl)methoxy)pyrimidin-2-yl)piperazine-1-carboxylate;
tert-butyl 4-(5-((5-(methylsulfonyl)pyridin-2-yl)methoxy)pyrimidin-2-yl)piperazine-1-carboxylate;
tert-butyl 4-(5-(3-fluoro-4-(methylsulfonyl)benzyloxy)pyrimidin-2-yl)piperazine-1-carboxylate;
tert-butyl 4-(5-(2-methyl-4-(methylsulfonyl)benzyloxy)pyrimidin-2-yl)piperazine-1-carboxylate;
(R)-tert-butyl 3-methyl-4-(5-((5-(methylsulfonyl)pyridin-2-yl)methoxy)pyrimidin-2-yl)piperazine-1-carboxylate;
(R)-tert-butyl 4-(5-(2-fluoro-4-(methylsulfonyl)benzyloxy)pyrimidin-2-yl)-3-methylpiperazine-1-carboxylate;
tert-butyl 4-(5-(2-fluoro-4-(methylsulfonyl)benzyloxy)pyrimidin-2-yl)piperazine-1-carboxylate;
tert-butyl 4-(5-(3-methyl-4-(methylsulfonyl)benzyloxy)pyrimidin-2-yl)piperazine-1-carboxylate;
tert-butyl 4-(5-((3-cyanopyridin-4-yl)methoxy)pyrimidin-2-yl)piperazine-1-carboxylate;
(R)-tert-butyl 4-(5-((3-cyanopyridin-4-yl)methoxy)pyrimidin-2-yl)-3-methylpiperazine-1-carboxylate;
4-((2-(4-(5-fluoropyrimidin-2-yl)piperazin-1-yl)pyrimidin-5-yloxy)methyl)nicotinonitrile;
(R)-4-((2-(4-(5-fluoropyrimidin-2-yl)-2-methylpiperazin-1-yl)pyrimidin-5-yloxy)methyl)nicotinonitrile;
5-fluoro-2-(4-(5-(4-(methylsulfonyl)benzyloxy)pyrimidin-2-yl)piperazin-1-yl)pyrimidine;
tert-butyl 4-(5-(4-((2-hydroxyethyl)(methyl)carbamoyl)benzyloxy)pyrimidin-2-yl)piperazine-1-carboxylate;
tert-butyl 4-(5-(4-(2-hydroxyethylcarbamoyl)benzyloxy)pyrimidin-2-yl)piperazine-1-carboxylate;
tert-butyl 4-(5-(4-(methylcarbamoyl)benzyloxy)pyrimidin-2-yl)piperazine-1-carboxylate;
tert-butyl 4-(5-(4-(isopropylcarbamoyl)benzyloxy)pyrimidin-2-yl)piperazine-1-carboxylate;
tert-butyl 4-(5-(4-(tert-butylcarbamoyl)benzyloxy)pyrimidin-2-yl)piperazine-1-carboxylate;
tert-butyl 4-(5-(4-(2-(dimethylamino)ethylcarbamoyl)benzyloxy)pyrimidin-2-yl)piperazine-1-carboxylate;
tert-butyl 4-(5-(4-((2-(dimethylamino)ethyl)(methyl)carbamoyl)benzyloxy)pyrimidin-2-yl)piperazine-1-carboxylate;
tert-butyl 4-(5-(4-(piperazine-1-carbonyl)benzyloxy)pyrimidin-2-yl)piperazine-1-carboxylate;
tert-butyl 4-(5-(4-(4-methylpiperazine-1-carbonyl)benzyloxy)pyrimidin-2-yl)piperazine-1-carboxylate;
tert-butyl 4-(5-(4-((1-methylpiperidin-4-yl)methylcarbamoyl)benzyloxy)pyrimidin-2-yl)piperazine-1-carboxylate;
tert-butyl 4-(5-(4-(morpholin-4-yl -1-carbonyl)benzyloxy)pyrimidin-2-yl)piperazine-1-carboxylate;
tert-butyl 4-(5-(4-(isopropylcarbamoyl)-3-methylbenzyloxy)pyrimidin-2-yl)piperazine-1-carboxylate;
tert-butyl 4-(5-(4-(tert-butylcarbamoyl)-3-methylbenzyloxy)pyrimidin-2-yl)piperazine-1-carboxylate;
isopropyl 4-(5-(4-(methylsulfonyl)benzyloxy)pyrimidin-2-yl)piperazine-1-carboxylate;
(R)-isopropyl 3-methyl-4-(5-(4-(methylsulfonyl)benzyloxy)pyrimidin-2-yl)piperazine-1-carboxylate;
isopropyl 4-(5-((3-cyanopyridin-4-yl)methoxy)pyrimidin-2-yl)piperazine-1-carboxylate;
(R)-isopropyl 4-(5-((3-cyanopyridin-4-yl)methoxy)pyrimidin-2-yl)-3-methylpiperazine-1-carboxylate;
1-methylcyclopropyl 4-(5-(4-(methylsulfonyl)benzyloxy)pyrimidin-2-yl)piperazine-1-carboxylate;
(R)-1-methylcyclopropyl 3-methyl-4-(5-(4-(methylsulfonyl)benzyloxy)pyrimidin-2-yl)piperazine-1-carboxylate;
1-methylcyclopropyl 4-(5-((3-cyanopyridin-4-yl)methoxy)pyrimidin-2-yl)piperazine-1-carboxylate;
cyclobutyl 4-(5-(4-(methylsulfonyl)benzyloxy)pyrimidin-2-yl)piperazine-1-carboxylate;
(R)-cyclobutyl 3-methyl-4-(5-(4-(methylsulfonyl)benzyloxy)pyrimidin-2-yl)piperazine-1-carboxylate;
cyclobutyl 4-(5-((3-cyanopyridin-4-yl)methoxy)pyrimidin-2-yl)piperazine-1-carboxylate;
1,1,1-trifluoropropan-2-yl 4-(5-(4-(methylsulfonyl)benzyloxy)pyrimidin-2-yl)piperazine-1-carboxylate;
(3R)-1,1,1-trifluoropropan-2-yl 3-methyl-4-(5-(4-(methylsulfonyl)benzyloxy)pyrimidin-2-yl)piperazine-1-carboxylate;
1,1,1-trifluoropropan-2-yl 4-(5-((3-cyanopyridin-4-yl)methoxy)pyrimidin-2-yl)piperazine-1-carboxylate;
2-cyanopropan-2-yl 4-(5-(4-(methylsulfonyl)benzyloxy)pyrimidin-2-yl)piperazine-1-carboxylate;
(R)-2-cyanopropan-2-yl 3-methyl-4-(5-(4-(methylsulfonyl)benzyloxy)pyrimidin-2-yl)piperazine-1-carboxylate;
2-cyanopropan-2-yl 4-(5-((3-cyanopyridin-4-yl)methoxy)pyrimidin-2-yl)piperazine-1-carboxylate;
(R)-2-cyanopropan-2-yl 4-(5-((3-cyanopyridin-4-yl)methoxy)pyrimidin-2-yl)-3-methylpiperazine-1-carboxylate;
oxetan-3-yl 4-(5-(4-(methylsulfonyl)benzyloxy)pyrimidin-2-yl)piperazine-1-carboxylate;
oxetan-3-yl 4-(5-((3-cyanopyridin-4-yl)methoxy)pyrimidin-2-yl)piperazine-1-carboxylate;
(3R)-tetrahydrofuran-3-yl 3-methyl-4-(5-(4-(methylsulfonyl)benzyloxy)pyrimidin-2-yl)piperazine-1-carboxylate;
3-methyloxetan-3-yl 4-(5-(4-(methylsulfonyl)benzyloxy)pyrimidin-2-yl)piperazine-1-carboxylate;
(R)-3-methyloxetan-3-yl 3-methyl-4-(5-(4-(methylsulfonyl)benzyloxy)pyrimidin-2-yl)piperazine-1-carboxylate;
3-methyloxetan-3-yl 4-(5-((3-cyanopyridin-4-yl)methoxy)pyrimidin-2-yl)piperazine-1-carboxylate;
(R)-tetrahydro-2H-pyran-4-yl 4-(5-((3-cyanopyridin-4-yl)methoxy)pyrimidin-2-yl)-3-methylpiperazine-1-carboxylate;
tert-butyl 4-(5-(4-(ethylsulfonyl)benzyloxy)pyrimidin-2-yl)piperazine-1-carboxylate;
tert-butyl 4-(5-(4-(cyclopropylsulfonyl)benzyloxy)pyrimidin-2-yl)piperazine-1-carboxylate;
4-((2-(4-(5-isopropyl-1,2,4-oxadiazol-3-yl)piperazin-1-yl)pyrimidin-5-yloxy)methyl)nicotinonitrile;
(R)-4-((2-(4-(5-isopropyl-1,2,4-oxadiazol-3-yl)-2-methylpiperazin-1-yl)pyrimidin-5-yloxy)methyl)nicotinonitrile;
5-isopropyl-3-(4-(5-(4-(methylsulfonyl)benzyloxy)pyrimidin-2-yl)piperazin-1-yl)-1,2,4-oxadiazole;
3-(4-(5-(4-(methylsulfonyl)benzyloxy)pyrimidin-2-yl)piperazin-1-yl)-5-(trifluoromethyl)-1,2,4-oxadiazole;
(R)-5-isopropyl-3-(3-methyl-4-(5-(4-(methylsulfonyl)benzyloxy)pyrimidin-2-yl)piperazin-1-yl)-1,2,4-oxadiazole;
3-(4-(5-(4-(methylsulfonyl)benzyloxy)pyrimidin-2-yl)piperazin-1-yl)-5-(trifluoromethyl)-1,2,4-oxadiazole;
tert-butyl 4-(5-(4-(trifluoromethylsulfinyl)benzyloxy)pyrimidin-2-yl)piperazine-1-carboxylate;
tert-butyl 4-(5-(4-(2-morpholinoethylsulfonyl)benzyloxy)pyrimidin-2-yl)piperazine-1-carboxylate;
tert-butyl 4-(5-(4-(methylsulfinyl)benzyloxy)pyrimidin-2-yl)piperazine-1-carboxylate;
tert-butyl 4-(5-((2-isobutyramidopyrimidin-5-yl)methoxy)pyrimidin-2-yl)piperazine-1-carboxylate;
tert-butyl 4-(5-((3-methylpyridin-4-yl)methoxy)pyrimidin-2-yl)piperazine-1-carboxylate;
(R)-4-((2-(4-(5-(difluoromethyl)-1,2,4-oxadiazol-3-yl)-2-methylpiperazin-1-yl)pyrimidin-5-yloxy)methyl)nicotinonitrile;
(R)-4-((2-(4-(5-cyclopropyl-1,2,4-oxadiazol-3-yl)-2-methylpiperazin-1-yl)pyrimidin-5- yloxy)methyl)nicotinonitrile;
(R)-4-((2-(4-(3-isopropyl-1,2,4-oxadiazol-5-yl)-2-methylpiperazin-1-yl)pyrimidin-5-yloxy)methyl)nicotinonitrile;
(R)-4-((2-(2-methyl-4-(3-(trifluoromethyl)-1,2,4-oxadiazol-5-yl)piperazin-1-yl)pyrimidin-5-yloxy)methyl)nicotinonitrile;
4-((2-((R)-4-(5-((S)-1-methoxyethyl)-1,2,4-oxadiazol-3-yl)-2-methylpiperazin-1-yl)pyrimidin-5-yloxy)methyl)nicotinonitrile; 4-((2-((R)-4-(5-((R)-1-methoxyethyl)-1,2,4-oxadiazol-3-yl)-2-methylpiperazin-1-yl)pyrimidin-5-yloxy)methyl)nicotinonitrile;
(R)-4-((2-(2-methyl-4-(5-(trifluoromethyl)-1,2,4-oxadiazol-3-yl)piperazin-1-yl)pyrimidin-5-yloxy)methyl)nicotinonitrile;
(R)—((R)-1,1,1-trifluoropropan-2-yl) 4-(5-((3-cyanopyridin-4-yl)methoxy)pyrimidin-2-yl)-3-methylpiperazine-1-carboxylate;
(R)—((S)-1,1,1-trifluoropropan-2-yl) 4-(5-((3-cyanopyridin-4-yl)methoxy)pyrimidin-2-yl)-3-methylpiperazine-1-carboxylate;
(R)-2,2,2-trifluoroethyl 4-(5-((3-cyanopyridin-4-yl)methoxy)pyrimidin-2-yl)-3-methylpiperazine-1-carboxylate;
2,2,2-trifluoroethyl 4-(5-((3-cyanopyridin-4-yl)methoxy)pyrimidin-2-yl)piperazine-1-is carboxylate;
(R)-4-((2-(2-methyl-4-(5-(3-methyloxetan-3-yl)-1,2,4-oxadiazol-3-yl)piperazin-1-yl)pyrimidin-5-yloxy)methyl)nicotinonitrile;
(R)-3-(trifluoromethyl)oxetan-3-yl 4-(5-(2-fluoro-4-(methylsulfonylmethyl)benzyloxy)-pyrimidin-2-yl)-3-methylpiperazine-1-carboxylate;
(R)-3-(trifluoromethyl)oxetan-3-yl 4-(5-(2-fluoro-4-(methylsulfonyl)benzyloxy)pyrimidin-2-yl)-3-methylpiperazine-1-carboxylate;
(R)-3-(trifluoromethyl)oxetan-3-yl 3-methyl-4-(5-(4-(methylsulfonyl)benzyloxy)pyrimidin-2-yl)piperazine-1-carboxylate; and
(R)-3-(trifluoromethyl)oxetan-3-yl 4-(5-((3-cyanopyridin-4-yl)methoxy)pyrimidin-2-yl)-3-methylpiperazine-1-carboxylate.

9) A compound as claimed in claim 1 selected from one or more of the following:
(R)-4-((2-(4-(5-isopropyl-1,2,4-oxadiazol-3-yl)-2-methylpiperazin-1-yl)pyrimidin-5-yloxy)methyl)nicotinonitrile;
(R)-4-((2-(4-(3-isopropyl-1,2,4-oxadiazol-5-yl)-2-methylpiperazin-1-yl)pyrimidin-5-yloxy)methyl)nicotinonitrile;
(R)-4-((2-(2-methyl-4-(3-(trifluoromethyl)-1,2,4-oxadiazol-5-yl)piperazin-1-yl)pyrimidin-5-yloxy)methyl)nicotinonitrile;
4-((2-((R)-4-(5-((S)-1-methoxyethyl)-1,2,4-oxadiazol-3-yl)-2-methylpiperazin-1-yl)pyrimidin-5-yloxy)methyl)nicotinonitrile;
4-((2-((R)-4-(5-((R)-1-methoxyethyl)-1,2,4-oxadiazol-3-yl)-2-methylpiperazin-1-yl)pyrimidin-5-yloxy)methyl)nicotinonitrile;
(R)—((R)-1,1,1-trifluoropropan-2-yl) 4-(5-((3-cyanopyridin-4-yl)methoxy)pyrimidin-2-yl)-3-methylpiperazine-1-carboxylate;
(R)—((S)-1,1,1-trifluoropropan-2-yl) 4-(5-((3-cyanopyridin-4-yl)methoxy)pyrimidin-2-yl)-3-methylpiperazine-1-carboxylate;
(R)-3-(trifluoromethyl)oxetan-3-yl 4-(5-(2-fluoro-4-(methylsulfonylmethyl)benzyloxy)-pyrimidin-2-yl)-3-methylpiperazine-1-carboxylate;
(R)-3-(trifluoromethyl)oxetan-3-yl 4-(5-(2-fluoro-4-(methylsulfonyl)benzyloxy)pyrimidin-2-yl)-3-methylpiperazine-1-carboxylate; and
(R)-3-(trifluoromethyl)oxetan-3-yl 4-(5-((3-cyanopyridin-4-yl)methoxy)pyrimidin-2-yl)-3-methylpiperazine-1-carboxylate.

10) A pharmaceutical formulation comprising a compound of formula I, as defined in any one of claims 1 to 9 and a pharmaceutically acceptable adjuvant, diluent or carrier.

11) A compound of formula I as claimed in any one of claims 1 to 9 for use as a medicament.

12) Use of a compound of formula I as defined in any one of claims 1 to 9 in the preparation of a medicament for the treatment or prophylaxis of conditions associated with obesity.

13) A compound as defined in any one of claims 1 to 9 for use in the treatment of diabetes.
